(19) United States
(12) Patent Application Publication
(10) Pub. No.: US 2022/0220520 A1
(43) Pub. Date: Jul. 14, 2022

(54) METHODS FOR PRODUCTION OF ERGOTHIONEINE

(71) Applicant: Danmarks Tekniske Universitet, Kgs. Lyngby (DK)

(72) Inventors: Irina BORODINA, Nivå (DK); Douglas Bruce KELL, Wrexham (GB); Steven van der HOEK, Kgs. Lyngby (DK); Behrooz Darbani SHIRVANEHDEH, Slagelse (DK)

(21) Appl. No.: 17/607,543

(22) PCT Filed: Apr. 29, 2020
(86) PCT No.: PCT/EP2020/061866

§ 371 Date: Oct. 29, 2021

(30) Foreign Application Priority Data
Apr. 30, 2019 (EP) 19171749.5

(51) Int. Cl. C12P 17/10; C07K 14/35; C12N 1/18; C12N 9/02; C12N 9/10; C12N 9/88

(52) U.S. Cl. CPC C12P 17/10; C07K 14/35; C12N 1/185; C12N 9/0083; C12R 2001/865; C12N 9/13; C12N 9/88; C12Y 201/01; C12N 9/1007

(57) ABSTRACT

The present invention relates to microbial factories, in particular yeast factories, for production of ergothioneine. Also provided are methods for producing ergothioneine in a yeast cell, as well as useful nucleic acids, polypeptides, vectors and host cells.

### TECHNICAL FIELD

The present invention relates to microbial factories, in particular yeast factories, for production of ergothioneine. Also provided are methods for producing ergothioneine in a yeast cell, as well as useful nucleic acids, polypeptides, vectors and host cells.

### BACKGROUND

Ergothioneine (ERG) (2-mercaptohistidine trimethylbetaine, (2S)-3-(2-Thioxo-2,3-dihydro-1H-imidazol-4-yl)-2-(trimethylammonio)propanoate) is a naturally occurring antioxidant that can be found universally in plants and mammals; it possesses a tautomeric structure, but is mainly present in the thione form at physiological pH. Ergothioneine displays antioxidant properties, including scavenging of free radicals and of reactive oxygen species, but also chelating of divalent metal ions. Ergothioneine has been shown to reduce oxidative damage in rats and humans.

So far only some bacteria and fungi have been identified as natural producers of ergothioneine. Ergothioneine was discovered in 1909 in the ergot fungus Claviceps purpurea, and its structure was determined two years later. Later, several other organisms were found to produce ergothioneine, including the filamentous fungus Neurospora crassa, the yeast Schizosaccharomyces pombe, and various actinobacteria including Mycobacterium smegmatis.

Humans must obtain ergothioneine through their diet; some mushrooms and other foods contain up to 7 mg·g⁻¹ dry weight. Because of its beneficial effects and possible involvement in preventing disease, ergothioneine is primed to take a place in the global dietary supplement market.

Studies show that ergothioneine in humans is mainly accumulated in the liver, the kidneys, in erythrocytes, bone marrow, the eye lens and seminal fluid. It is transported by SLC22A4 (previously known as OCTN1), a transporter common to most animals. The high abundance of ergothioneine in the body could indicate that ergothioneine is involved in the maintenance of health or the mitigation of disease. Ergothioneine has demonstrated effects in in vivo models of several neurodegenerative diseases, in ischaemia reperfusion injury, and in a variety of other diseases. It is also reported that ergothioneine can accumulate at sites of injury through the upregulation of SLC22A4/OCTN1. Ergothioneine is only slowly metabolized and excreted in humans, again suggesting that it plays an important role in the body.

Ergothioneine is synthesized from one molecule of L-histidine, one molecule of cysteine, and 3 methyl groups donated via S-adenosyl-L-methionine (FIG. 1). In M. smegmatis, the reaction sequence is catalyzed by 5 enzymes, encoded by EgtA, EgtB, EgtC, EgtD and EgtE genes positioned together in a cluster. Four enzymes of the cluster EgtA, EgtB, EgtC, and EgtD catalyze 4 individual reactions that produce 5-(hercyn-2-yl)-L-cysteine S-oxide (HCO) intermediate. In fungi, the biosynthetic pathway is different, as a single enzyme Egt1 catalyzes the methylation of histidine to give hercynine, which in turn is sulfoxidized with cysteine, producing HCO. HCO is converted into 2-(hydroxysulfanyl)hercynine by β-lyase, encoded by EgtE in M. smegmatis and by Egt2 gene in fungi. This compound is apparently spontaneously reduced to ergothioneine.

Current methods for production of ergothioneine are mostly based on chemical synthesis. Such methods are not cost-effective and also have a significant impact on the environment. Therefore, methods for cost-effective and environmental-friendly production of ergothioneine are required.

### SUMMARY

The present invention provides yeast cells capable of producing ergothioneine and methods for ergothioneine production in a yeast cell.

In one aspect is provided a yeast cell capable of producing ergothioneine, said yeast cell expressing:
a) at least one first heterologous enzyme capable of converting L-histidine and/or L-cysteine to S-(hercyn-2-yl)-L-cysteine-S-oxide; and
b) at least one second heterologous enzyme capable of converting S-(hercyn-2-yl)-L-cysteine-S-oxide to 2-(hydroxysulfanyl)-hercynine;

wherein the yeast cell is further capable of converting 2-(hydroxysulfanyl)-hercynine to ergothioneine.

Also provided herein are methods for producing ergothioneine in a yeast cell, comprising the steps of:
i) providing a yeast cell capable of producing ergothioneine, said yeast cell expressing:
   a) at least one first heterologous enzyme capable of converting L-histidine and/or L-cysteine to S-(hercyn-2-yl)-L-cysteine-S-oxide; and
   b) at least one second heterologous enzyme capable of converting S-(hercyn-2-yl)-L-cysteine-S-oxide to 2-(hydroxysulfanyl)-hercynine;
      wherein the yeast cell is further capable of converting 2-(hydroxysulfanyl)-hercynine to ergothioneine;
ii) incubating said yeast cell in a medium;

thereby obtaining ergothioneine.

Also provided herein are:
a polypeptide having the sequence as set forth in SEQ ID NO: 6 (CpEgt1) or a functional variant thereof having at least 70% homology to SEQ ID NO: 6, homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto;
a polypeptide having the sequence as set forth in SEQ ID NO: 12 (CpEgt2) or a functional variant thereof having at least 70% homology to SEQ ID NO: 12, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Also provided herein are:
a nucleic acid having the sequence as set forth in SEQ ID NO: 5 or SEQ ID NO: 16, or has at least 70% homology to SEQ ID NO: 5 or SEQ ID NO: 16, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto;
a nucleic acid having the sequence as set forth in SEQ ID NO: 11 or SEQ ID NO: 18, or has at least 70% homology to SEQ ID NO: 11 or SEQ ID NO: 18, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Also provided are vectors comprising the above nucleic acids, as well as host cells comprising said vectors and/or said nucleic acids or polypeptides.

Also provided is the use of above polypeptides, nucleic acids, vectors or host cells for the production of ergothioneine.

### DESCRIPTION OF THE DRAWINGS

FIG. 1: Pathway of ergothioneine biosynthesis in bacteria and fungi. SAM=S-adenosyl-L-methioneine, SAH=S-adenosyl-L-homocysteine, γ-GC=γ-L-glutamyl-L-cysteine, γ-GHCO=γ-L-glutamyl-S-(hercyn-2-yl)-L-cysteine S-oxide, HCO=S-(hercyn-2-yl)-L-cysteine S-oxide, 2-HSH=2-(hydroxysulfanyl)hercynine.

FIG. 2: Ergothioneine production in strains with integrated ergothioneine biosynthesis pathway. The strains have various combinations of genes from different organisms, as indicated. Black boxes: intracellular ergothioneine; white boxes: extracellular ergothioneine. Y axis represents ergothioneine production in mg/L. 1: SC+20 g/l glucose+1 g/l His/Cys/Met (Batch medium), 48 hours; 2: SC+40 g/l glucose (Batch medium), 72 hours; 3: SC+60 g/l EnPump substrate, 0.6% reagent A (Fed batch medium), 72 hours. SC=Synthetic Complete

FIG. 3: Production of ergothioneine over time in the production strain with or without transporters MsErgT or HsSCL22A4 (Hs.SCL22A4X on the figure) under different conditions. Black boxes: intracellular ergothioneine; white boxes: extracellular ergothioneine. 1: SC+20 g/l glucose+1 g/l His/Cys/Met (Batch medium), 48 hours; 2: SC+40 g/l glucose (Batch medium), 72 hours; 3: SC+60 g/l EnPump substrate, 0.6% reagent A (Fed batch medium), 72 hours. SC=Synthetic Complete

FIG. 4: Striped boxes: intracellular ergothioneine; black boxes; extracellular ergothioneine; black line: OD. (A): ST8461 in SC+40 g/L glucose. (B): ST8461 in SC+40 g/L glucose+1 g/L aa. (C): ST8461 in SC+40 g/L glucose+2 g/L aa. (D): ST8654 in SC+40 g/L glucose. (E) ST8654 in SC+40 g/L glucose+1 g/L aa. (F): ST8654 in SC+40 g/L glucose+2 g/L aa. SC=Synthetic Complete

FIG. 5: Percentage of PI stained cells for control (Y axis) in the indicated strains with the transporter in media without 1 g/l histidine, cysteine and methionine (striped boxes) versus media with 1 g/l histidine, cysteine and methionine (black boxes). (A): ST7574. (B): ST8654. (C): ST8461. SC=Synthetic Complete

FIG. 6: Ergothioneine production by ST8927 during fed-batch cultivation under carbon limited conditions. N═(NH₄)₂SO₄, Mg═MgSO₄, tm=trace metals, vit=vitamins.

FIG. 7: Ergothioneine production in strains with integrated ergothioneine biosynthesis pathway (two copies of NcEgt1 and SpEgt2). Besides the integrated ergothioneine biosynthesis pathway, the strains carry an additional modification of a gene, as indicated in the figure. Y axis represents total ergothioneine production in mg/L.

FIG. 8: Ergothioneine production in strains with integrated ergothioneine biosynthesis pathway (two copies of NcEgt1 and SpEgt2). The strains have various combinations of modified genes, as indicated in the figure. Y axis represents total ergothioneine production in mg/L. TRA res.=TRA resistance.

FIG. 9: Ergothioneine production in strains with integrated ergothioneine biosynthesis pathway (two copies of NcEgt1 and SpEgt2). The strains have various combinations of modified genes, as indicated in the figure. Y axis represents total ergothioneine production in mg/L. TRA res.=TRA resistance.

FIG. 10: Ergothioneine production in strains with integrated ergothioneine biosynthesis pathway (two copies of NcEgt1 and SpEgt2). Besides the integrated ergothioneine biosynthesis pathway, the strains carry an additional modification of a gene, as indicated in the figure. Black boxes: intracellular ergothioneine; white boxes: extracellular ergothioneine. Thus, Y axis represents intracellular and extracellular ergothioneine production in mg/L.

FIG. 11: Ergothioneine production in strains with integrated ergothioneine biosynthesis pathway (one copy of NcEgt1 and SpEgt2). Besides the integrated ergothioneine biosynthesis pathway, the strains carry an additional modification of a gene, as indicated in the figure. Y axis represents total ergothioneine production in mg/L. TRA res.=TRA resistance.

FIG. 12: Ergothioneine production in strain ST8460 S. cerevisiae, ST9584 Y. lipolytica and ST9703 Y. lipolytica. Black bars: Glucose: ergothioneine production under batch conditions (SC medium with 20 g/L glucose); white bars: FiT: ergothioneine production under stimulated fed-batch conditions (SC medium with 60 g/L Enpump substrate+0.6% reagent A). Y axis represents total ergothioneine production in mg/L. SC=Synthetic Complete.

FIG. 13: Ergothioneine production using varying starting cell dry weight concentrations and varying concentrations of reagent A as indicated on the X axis. Y axis represents total ergothioneine production in mg/L.

FIG. 14: Ergothioneine and histidine production in selected strains. Strains were grown in media containing 0.25 mM β-(1,2,4-triazol-3-yl)-DL-alanine. Black boxes: histidine; white boxes: ergothioneine. Y axis represents total ergothioneine and histidine production in mg/L.

### DETAILED DESCRIPTION OF THE INVENTION

The present disclosure relates to yeast cells and methods for production of ergothioneine.

Yeast Cell

The present disclosure relates to a yeast cell capable of producing ergothioneine. Herein is thus provided a yeast cell capable of producing ergothioneine, said yeast cell expressing:
a) at least one first heterologous enzyme capable of converting L-histidine and/or L-cysteine to S-(hercyn-2-yl)-L-cysteine-S-oxide; and
b) at least one second heterologous enzyme capable of converting S-(hercyn-2-yl)-L-cysteine-S-oxide to 2-(hydroxysulfanyl)-hercynine;

wherein the yeast cell is further capable of converting 2-(hydroxysulfanyl)-hercynine to ergothioneine.

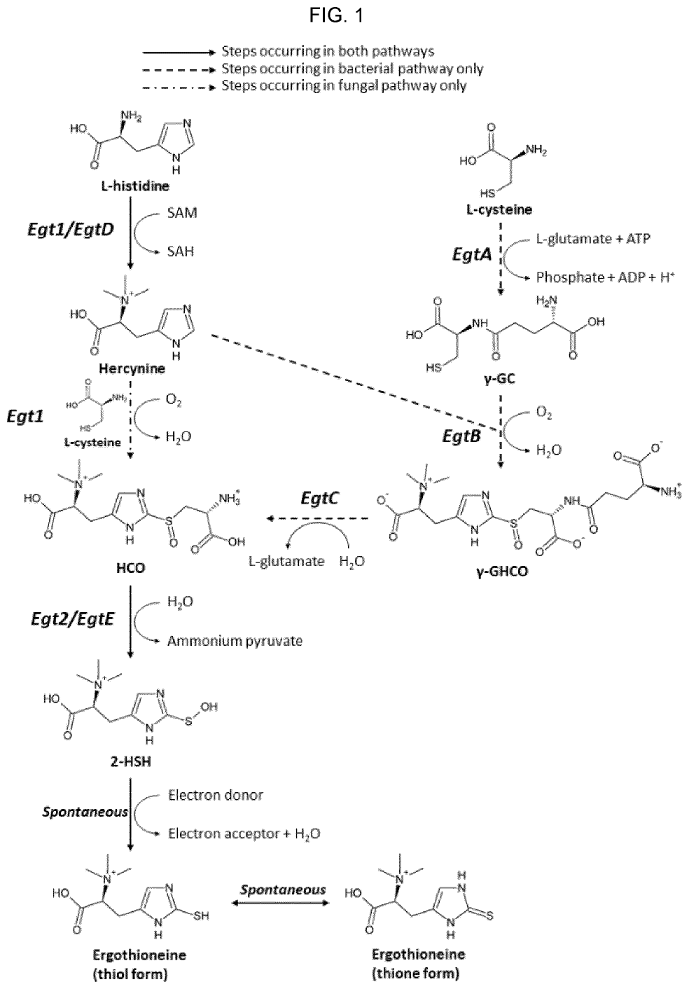

The yeast cells disclosed herein are thus all capable of converting 2-(hydroxysulfanyl)-hercynine to ergothioneine. This can be because the yeast cell natively (i.e. without modifications) has the ability to convert 2-(hydroxysulfanyl)-hercynine to ergothioneine, or because the yeast cell has been engineered to gain that ability, as is known in the art. Generally, cells, including yeast cells, have the ability of spontaneously converting 2-(hydroxysulfanyl)-hercynine to ergothioneine, particularly to ergothioneine in the thiol form, which then spontaneously can be converted to ergothioneine in the thione form, and vice versa. The spontaneous conversion of 2-(hydroxysulfanyl)-hercynine to ergothioneine requires an electron donor, and releases an electron acceptor and H₂O (FIG. 1).

The yeast cells of the present disclosure preferably are capable of synthesising L-histidine and L-cysteine.

In some embodiments, the yeast cell is a cell from a GRAS (Generally Recognized As Safe) organism or a non-pathogenic organism or strain.

In some embodiments, the genus of said yeast is selected from Saccharomyces, Pichia, Yarrowia, Kluyveromyces, Candida, Rhodotorula, Rhodosporidium, Cryptococcus, Schizosaccharomyces, Trichosporon and Lipomyces. In some preferred embodiments, the genus of said yeast is Saccharomyces, Pichia, Kluyveromyces or Yarrowia.

The yeast cell may be selected from the group consisting of Saccharomyces cerevisiae, Pichia pastoris, Komagataella phaffii, Kluyveromyces marxianus, Kluyveromyces lactis, Schizosaccharomyces pombe, Cryptococcus albidus, Lipomyces lipofera, Lipomyces starkeyi, Rhodosporidium toruloides, Rhodotorula glutinis, Trichosporon pullulan and Yarrowia lipolytica. In preferred embodiments, the yeast cell is a Kluyveromyces marxianus cell, a Saccharomyces cerevisiae cell or a Yarrowia lipolytica cell; preferably the yeast cell is a Saccharomyces cerevisiae cell.

First Heterologous Enzyme

The first heterologous enzyme expressed in the yeast cell is capable of converting L-histidine and/or L-cysteine to S-(hercyn-2-yl)-L-cysteine-S-oxide. The first heterologous enzyme is not natively expressed in the yeast cell. It may be derived from a eukaryote or a prokaryote, as detailed below.

Enzymes capable of catalysing the above reaction are: L-histidine Nα-methyltransferases (EC 2.1.1.44), hercynylcysteine S-oxide synthase (EC 1.14.99.51), glutamate-cysteine ligases (EC 6.3.2.2), γ-glutamyl hercynylcysteine S-oxide synthases (EC 1.14.99.50), and γ-glutamyl hercynylcysteine S-oxide hydrolases (EC 3.5.1.118). In some embodiments, the first heterologous enzyme is an enzyme having an EC number selected from EC 2.1.1.44, EC 1.14.99.51, EC 6.3.2.2, EC 1.14.99.50 and EC 3.5.1.118. In one embodiment, the EC number is 2.1.1.44. In another embodiment, the EC number is EC 1.14.99.51.

L-histidine Nα-methyltransferases (EC 2.1.1.44), also termed dimethylhistidine N-methyltransferases, catalyse the reaction:

3 S-adenosyl-L-methionine+L-histidine⇔3 S-adenosyl-L-homocysteine+hercynine.

Using Fe²⁺ as cofactor. Such enzymes thus need L-histidine as a substrate.

Hercynylcysteine S-oxide synthase (EC 1.14.99.51) catalyse the reaction:

Hercynine+L-cysteine+O₂⇔S-hercyn-2-yl-L-cysteine S-oxide+H₂O

Using Fe²⁺ as cofactor. Such enzymes need L-cysteine as a substrate.

Glutamate-cysteine ligases (EC 6.3.2.2) catalyse the reaction:

Hercynine+L-cysteine+O₂⇔S-hercyn-2-yl-L-cysteine S-oxide+H₂O

Using Fe²⁺ as cofactor. Such enzymes need L-cysteine as a substrate.

γ-glutamyl hercynylcysteine S-oxide synthases (EC 1.14.99.50) catalyse the reaction:

Hercynine+L-cysteine+O₂⇔S-hercyn-2-yl-L-cysteine S-oxide+H₂O

Using Fe²⁺ as cofactor. Such enzymes need L-cysteine as a substrate.

γ-glutamyl hercynylcysteine S-oxide hydrolases (EC 3.5.1.118) catalyse the reaction:

Hercynine+L-cysteine+O₂⇔S-hercyn-2-yl-L-cysteine S-oxide+H₂O

Using Fe²⁺ as cofactor. Such enzymes need L-cysteine as a substrate.

Throughout this disclosure, it will be understood that if the first heterologous enzyme is a hercynylcysteine S-oxide synthase (EC 1.14.99.51), a glutamate-cysteine ligase (EC 6.3.2.2), a γ-glutamyl hercynylcysteine S-oxide synthase (EC 1.14.99.50), or a γ-glutamyl hercynylcysteine S-oxide hydrolase (EC 3.5.1.118), then the yeast cell needs L-cysteine as a substrate. If the first heterologous enzyme is an L-histidine Nα-methyltransferase (EC 2.1.1.44), also termed dimethylhistidine N-methyltransferase, then the yeast cell needs L-histidine as a substrate.

In some embodiments, the first heterologous enzyme is Egt1, derived from a eukaryote such as a fungus, for example a yeast. The yeast cell of the present disclosure may, in addition to the first heterologous enzyme, natively express an enzyme capable of catalysing the same reaction as the first heterologous enzyme, or the yeast cell may be devoid of enzyme capable of catalysing this reaction. An enzyme, in particular a first heterologous enzyme, is derived from an organism if it is natively found in said organism.

In some embodiments, the first heterologous enzyme is derived from a eukaryote and is classified as EC 2.1.1.44 and/or EC.1.14.99.51.

In some embodiments, the first heterologous enzyme is Egt1 from Neurospora crassa, Claviceps purpurea, Schizosaccharomyces pombe, Rhizopus stolonifera, Aspergillus nidulans, Aspergillus niger, Penicillium roqueforti, Penicillium notatum, Sporobolomyces salmonicolor, Aspergillus oryzae, Aspergillus carbonarius, Neurospora tetrasperma, Agaricus bisporus, Pleurotus ostreatus, Lentinula edodes or Grifola frondosa, or a functional variant thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. The term “functional variant” refers to variants such as mutants, which retain total or partial activity and are still capable of converting L-histidine and/or L-cysteine to S-(hercyn-2-yl)-L-cysteine-S-oxide. The skilled person knows how to determine whether a functional variant retains said activity, for example by detecting the products using liquid chromatography, optionally coupled to mass spectrometry.

The accession numbers of above-listed Egt1 enzymes are listed in Table A below.

TABLE A
Egt1 from fungal organisms and GenBank accession numbers.

| Organism (fungi) | GenBank Accession number |
|---|---|
| Neurospora crassa (Ncas) | XP_956324.3 |
| Claviceps purpurea (Cpur) | CCE33591.1 |
| Schizosaccharomyces pombe (Spom) | NP_596639.2 |
| Rhizopus stolonifera (Rsto) | RCH97401.1 |
| Aspergillus nidulans (Anid) | XP_680889.1 |
| Aspergillus niger (Anig) | XP_001397117.2 |
| Penicillium roqueforti (Proq) | CDM31097.1 |
| Penicillium notatum (Pnot) | KZN88090.1 |
| Sporobolomyces salmonicolor (Ssal) | CEQ42739.1 |
| Aspergillus oryzae (Aory) | XP_001727309.1 |
| Aspergillus carbonarius (Acar) | OOF91620.1 |
| Neurospora tetrasperma (Ntet) | XP_009849693.1 |
| Agaricus bisporus (Abis) | XP_006462499.1 |
| Pleurotus ostreatus (Post) | KDQ26018.1 |
| Lentinula edodes (Ledo) | GAW05586.1 |
| Grifola frondosa (Gfro) | OBZ71212.1 |

In some embodiments, the first heterologous enzyme is derived from Neurospora crassa, Schizosaccharomyces pombe, or Claviceps purpurea. The sequences of the corresponding Egt1 enzymes are set forth in SEQ ID NO: 2 (N. crassa), SEQ ID NO: 4 (S. pombe) and SEQ ID NO: 6 (C. purpurea).

In particular embodiments, the first heterologous enzyme is selected from the group consisting of: NcEgt1 (SEQ ID NO: 2), SpEgt1 (SEQ ID NO: 4) and CpEgt1 (SEQ ID NO: 6), and functional variants thereof having at least 70% homology to SEQ ID NO: 2, SEQ ID NO: 4 or SEQ ID NO: 6, %, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Second Heterologous Enzyme

The second heterologous enzyme expressed in the yeast cell is capable of converting S-(hercyn-2-yl)-L-cysteine-S-oxide to 2-(hydroxysulfanyl)-hercynine. In particular, the second heterologous enzyme is capable of converting the S-(hercyn-2-yl)-L-cysteine-S-oxide produced by the first heterologous enzyme to 2-(hydroxysulfanyl)-hercynine.

Enzymes capable of catalysing the above reaction are: β-lyases and hercynylcysteine sulfoxide lyases, also termed hercynylcysteine S-oxide synthases (EC 4.4.1.-). Thus, in some embodiments, the second heterologous enzyme is a β-lyase or a hercynylcysteine sulfoxide lyase (EC 4.4.1.-).

Such enzymes can catalyse the reaction:

Hercynine+L-cysteine+O₂⇔S-hercyn-2-yl-L-cysteine S-oxide+H₂O

Using Fe²⁺ as cofactor.

In some embodiments, the second heterologous enzyme is Egt2, derived from a eukaryote such as a fungus, for example a yeast. The yeast cell of the present disclosure may, in addition to the first heterologous enzyme, natively express an enzyme capable of catalysing the same reaction as the second heterologous enzyme, or the yeast cell may be devoid of enzyme capable of catalysing this reaction. In some embodiments, the second heterologous enzyme is EgtE, derived from a bacterium. An enzyme, in particular a second heterologous enzyme, is derived from an organism if it is natively found in said organism.

In some embodiments, the second heterologous enzyme is Egt2 from Neurospora crassa, Claviceps purpurea, Schizosaccharomyces pombe, Rhizopus stolonifera, Aspergillus nidulans, Aspergillus niger, Penicillium roqueforti, Penicillium notatum, Sporobolomyces salmonicolor, Aspergillus oryzae, Aspergillus carbonarius, Neurospora tetrasperma, Agaricus bisporus, Pleurotus ostreatus, Lentinula edodes, Grifola frondosa, Ganoderma lucidum, or Cantharellus cibarius, or a functional variant thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. The term “functional variant” refers to variants such as mutants, which retain total or partial activity and are still capable of converting S-(hercyn-2-yl)-L-cysteine-S-oxide to 2-(hydroxysulfanyl)-hercynine. The skilled person knows how to determine whether a functional variant retains said activity, for example by detecting the products using liquid chromatography, optionally coupled to mass spectrometry.

In other embodiments, the second heterologous enzyme is a bacterial EgtE, such as EgtE from Mycobacterium smegmatis, Nocardia asteroids, Streptomyces albus, Streptomyces fradiae, Streptomyces griseus, Actinoplanes philippinensis, Aspergillus fumigatus, Mycobacterium tuberculosis, Mycobacterium kansasii, Mycobacterium intracellulare, Mycobacterium fortuitum, Mycobacterium ulcerans, Mycobacterium balnei, Mycobacterium leprae, Mycobacterium avium, Mycobacterium bovis, Mycobacterium marinum, Mycobacterium microti, Mycobacterium paratuberculosis, Mycobacterium phlei, Rhodococcus rhodocrous (Mycobacterium rhodocrous), Arthrospira platensis, Arthrospira maxima, Aphanizomenon flos-aquae, Scytonema sp., Oscillatoria sp. and Rhodophyta sp., or a functional variant thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. The term “functional variant” refers to variants such as mutants, which retain total or partial activity and are still capable of converting S-(hercyn-2-yl)-L-cysteine-S-oxide to 2-(hydroxysulfanyl)-hercynine. The skilled person knows how to determine whether a functional variant retains said activity, for instance using liquid chromatography to detect the products, optionally coupled to mass spectrometry.

The accession numbers of above-listed Egt2 and EgtE enzymes are listed in Table B below.

TABLE B
Egt2 from fungal organisms, EgtE from bacterial
organisms, and GenBank accession numbers.

| Organism (fungi) | Egt2 |
|---|---|
| Neurospora crassa (Ncas) | XP_001728131.1 |
| Claviceps purpurea (Cpur) | CCE33140.1 |
| Schizosaccharomyces pombe (Spom) | NP_595091.1 |
| Rhizopus stolonifera (Rsto) | RCI05990.1 |
| Aspergillus nidulans (Anid) | XP_663831.1 |
| Aspergillus niger (Anig) | XP_001390787.2 |
| Penicillium roqueforti (Proq) | CDM34493.1 |
| Penicillium notatum (Pnot) | KZN85331.1 |
| Sporobolomyces salmonicolor (Ssal) | CEQ41088.1 |
| Aspergillus oryzae (Aory) | XP_001821768.1 |
| Aspergillus carbonarius (Acar) | OOF99450.1 |
| Neurospora tetrasperma (Ntet) | XP_009848922.1 |
| Agaricus bisporus (Abis) | XP_006461570.1 |
| Pleurotus ostreatus (Post) | KDQ26326.1 |
| Lentinula edodes (Ledo) | GAV99896.1 |
| Grifola frondosa (Gfro) | OBZ72541.1 |
| Ganoderma lucidum (Gluc) | AUN37957.1 |
| Cantharellus cibarius (Ccib) | AWA82152.1 |
| Mycobacterium smegmatis (Msme) | WP_011731155.1 |
| Nocardia asteroids (Nast) | WP_022566259.1 |
| | Multispecies |
| Streptomyces albus (Salb) | WP_030543061.1 |
| Streptomyces fradiae (Sfra) | WP_070159474.1 |
| Streptomyces griseus (Sgri) | WP_030191586.1 |
| | Multispecies |
| Actinoplanes philippinensis (Aphi) | WP_093610803.1 |
| Aspergillus fumigatus (Afum) | XP_754202.1 |
| Mycobacterium tuberculosis (Mtur) | WP_079029600.1 |
| Mycobacterium kansasii (Mkan) | WP_103802346.1 |
| Mycobacterium intracellulare (Mint) | WP_014941167.1 |
| Mycobacterium forfuitum (Mfor) | WP_076203140.1 |
| Mycobacterium ulcerans (Mulc) | WP_096369529.1 |
| Mycobacterium balnei (Mbal) | WP_117431391.1 |
| Mycobacterium leprae (Mlep) | WP_041323321.1 |
| Mycobacterium avium (Mavi) | WP_044543419.1 |
| Mycobacterium bovis (Mbov) | WP_003901701.1 |
| | Multispecies |
| Mycobacterium marinum (Mmar) | WP_117431391.1 |
| Mycobacterium microti (Mmic) | PLV46245.1 |
| Mycobacterium paratuberculosis (Mpar) | WP_003877001.1 |
| Mycobacterium phlei (Mphl) | WP_003888643.1 |
| Rhodococcus rhodocrous (Rrho) | WP_006938916.1 |
| Reclassified Mycobacterium | Multispecies |
| rhodocrous | |
| Arthrospira platensis (Apla) | WP_062945872.1 |
| Arthrospira maxima (Amax) | WP_006621917.1 |
| | Multispecies |
| Aphanizomenon flos-aquae (Aflo) | WP_039201356.1 |
| Scytonema sp. | WP_073633333.1 |
| | WP_048869496.1 |
| Oscillatoria sp. | WP_044196545.1 |
| | WP_015175683.1 |
| Rhodophyta sp. | OSX68822.1 |
| | XP_005703716.1 |

In some embodiments, the second heterologous enzyme is derived from Neurospora crassa, Schizosaccharomyces pombe, Claviceps purpurea or Mycobacterium smegmatis. The sequences of the corresponding Egt2 or EgtE enzymes are set forth in SEQ ID NO: 8 (N. crassa), SEQ ID NO: 10 (S. pombe), SEQ ID NO: 12 (C. purpurea) and SEQ ID NO: 14 (M. smegmatis).

In particular embodiments the second heterologous enzyme expressed in the yeast cell may be selected from NcEgt2 (SEQ ID NO: 8), SpEgt2 (SEQ ID NO: 10), CpEgt2 (SEQ ID NO: 12), and MsEgtE (SEQ ID NO: 14), and functional variants thereof having at least 70% homology to SEQ ID NO: 8, SEQ ID NO: 10, SEQ ID NO: 12 or SEQ ID NO: 14, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Combinations of First and Second Heterologous Enzymes

Although all combinations of the first and second heterologous enzymes disclosed herein may be useful for providing a yeast factory for production of ergothioneine, specific combinations of first and second heterologous enzymes may be of particular interest in the context of the present invention.

In some embodiments, the first and the second heterologous enzymes are:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2;
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In specific embodiments, the yeast cell expresses a first and second heterologous enzymes as follows:
i) NcEgt1 and CpEgt2;
ii) NcEgt1 and SpEgt2;
iii) NcEgt1 and NcEgt2;
iv) NcEgt1 and MsEgtE;
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cells of the invention express a first and a second heterologous enzymes which are not:
iii) NcEgt1 and NcEgt2; or
viii) SpEgt1 and MsEgtE; or
x) CpEgt1 and SpEgt2.

Nucleic Acids Encoding the First and Second Heterologous Enzymes

Yeast cells useful in the context of the present disclosure can be engineered as is known in the art. For example, expression of the first and second heterologous enzymes can be achieved by introducing in the yeast cell nucleic acids encoding them. Such nucleic acids may be codon-optimised to improve expression in the yeast cell, as is known in the art.

In some embodiments, the first heterologous enzyme is derived from Neurospora crassa, Schizosaccharomyces pombe, or Claviceps purpurea. The sequences of the corresponding Egt1 enzymes are set forth in SEQ ID NO: 2 (N. crassa), SEQ ID NO: 4 (S. pombe) and SEQ ID NO: 6 (C. purpurea). The corresponding nucleic acid sequences are set forth in SEQ ID NO: 1 or SEQ ID NO: 15 (N. crassa), SEQ ID NO: 3 (S. pombe) and SEQ ID NO: 5 or SEQ ID NO: 16 (C. purpurea). Such nucleic acids, or variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, may thus suitably be introduced in the yeast cell, either in the genome or as part of a vector suitable for expression, as is known in the art.

In some embodiments, the second heterologous enzyme is derived from Neurospora crassa, Schizosaccharomyces pombe, Claviceps purpurea or Mycobacterium smegmatis. The sequences of the corresponding Egt2 or EgtE enzymes are set forth in SEQ ID NO: 8 (N. crassa), SEQ ID NO: 10 (S. pombe), SEQ ID NO: 12 (C. purpurea) and SEQ ID NO: 14 (M. smegmatis). The corresponding nucleic acid sequences are set forth in SEQ ID NO: 7 or SEQ ID NO: 17 (N. crassa), SEQ ID NO: 9 (S. pombe), SEQ ID NO: 11 or SEQ ID NO: 18 (C. purpurea) and SEQ ID NO: 13 or SEQ ID NO: 19 (M. smegmatis). Such nucleic acids, or variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, may thus suitably be introduced in the yeast cell, either in the genome or as part of a vector suitable for expression, as is known in the art.

In specific embodiments, nucleic acids or homologues thereof having at least 70% homology thereto are introduced in the yeast cell as shown below:
i) NcEgt1 and CpEgt2: SEQ ID NO: 1 or 15 and SEQ ID NO: 11 or 18;
ii) NcEgt1 and SpEgt2: SEQ ID NO: 1 or 15 and SEQ ID NO: 9;
iii) NcEgt1 and NcEgt2: SEQ ID NO: 1 or 15 and SEQ ID NO: 7 or 17;
iv) NcEgt1 and MsEgtE: SEQ ID NO: 1 or 15 and SEQ ID NO: 13 or 19;
v) SpEgt1 and NcEgt2: SEQ ID NO: 3 and SEQ ID NO: 7 or 17
vi) SpEgt1 and SpEgt2: SEQ ID NO: 3 and SEQ ID NO: 9;
vii) SpEgt1 and CpEgt2: SEQ ID NO: 3 and SEQ ID NO: 11 or 18;
viii) SpEgt1 and MsEgtE: SEQ ID NO: 3 and SEQ ID NO: 13 or 19;
ix) CpEgt1 and NcEgt2: SEQ ID NO: 5 or 16 and SEQ ID NO: 7 or 17;
x) CpEgt1 and SpEgt2: SEQ ID NO: 5 or 16 and SEQ ID NO: 9;
xi) CpEgt1 and CpEgt2: SEQ ID NO: 5 or 16 and SEQ ID NO: 11 or 18;
xii) CpEgt1 and MsEgtE: SEQ ID NO: 5 or 16 and SEQ ID NO: 13 or 19.

In specific embodiments, nucleic acids as shown in i), ii), iv) or xii) above or homologues having at least 70% homology thereto are introduced. In some embodiments, the nucleic acids introduced are not the nucleic acids shown in iii), viii) or x) above.

Ergothioneine Transporter

In some embodiments, the yeast cell is capable of secreting at least part of the ergothioneine it produces. The yeast cell may natively be able to do so, or it may be further modified to improve secretion. This can be done by expression or overexpression of an ergothioneine transporter, in particular a heterologous ergothioneine transporter.

Thus in some embodiments, the yeast cell further expresses the ergothioneine transporter of M. smegmatis as set forth in SEQ ID NO: 35 (MsErgT) or the ergothioneine transporter of H. sapiens as set forth in SEQ ID NO: 36 (HsSLC22A4) or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. A functional variant here refers to variants such as mutants which retain total or partial ergothioneine transporter activity. The skilled person knows how to determine whether a functional variant retains said activity.

In some embodiments, the yeast cell expresses an ergothioneine transporter such as MsErgT as set forth in SEQ ID NO: 35 or HsSLC22A4 as set forth in SEQ ID NO: 36 or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In specific embodiments, the yeast cell expresses an ergothioneine transporter such as MsErgT as set forth in SEQ ID NO: 35 or HsSLC22A4 as set forth in SEQ ID NO: 36 or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cell expresses an ergothioneine transporter such as MsErgT as set forth in SEQ ID NO: 35 or HsSLC22A4 as set forth in SEQ ID NO: 36 or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes which are not:
iii) NcEgt1 and NcEgt2; or
viii) SpEgt1 and MsEgtE; or
x) CpEgt1 and SpEgt2.

In specific embodiments, the yeast cell expresses an ergothioneine transporter such as MsErgT as set forth in SEQ ID NO: 35 and/or HsSLC22A4 as set forth in SEQ ID NO: 36 or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses two copies of NcEgt1 and two copies of CpEgt2.

In some embodiments, the yeast cell further expresses the ergothioneine transporter of Arabidopsis thaliana as set forth in SEQ ID NO: 37 (AtOCT1), or the ergothioneine transporter of S. cerevisiae as set forth in SEQ ID NO: 39 (ScAQR1) or the ergothioneine transporter of H. sapiens as set forth in SEQ ID NO: 41 (HsSLC22A16) or as set forth in SEQ ID NO: 43 (HsSLC22A32) or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. A functional variant here refers to variants such as mutants which retain total or partial ergothioneine transporter activity. The skilled person knows how to determine whether a functional variant retains said activity.

The gene encoding AtOCT1 is set forth in SEQ ID NO: 38.

The gene encoding ScAQR1 is set forth in SEQ ID NO: 40.

The gene encoding HsSLC22A16 is set forth in SEQ ID NO: 42.

The gene encoding HsSLC22A32 is set forth in SEQ ID NO: 44.

In some embodiments, the yeast cell expresses an ergothioneine transporter such as AtOCT1 as set forth in SEQ ID NO:37, ScAQR1 as set forth in SEQ ID NO:39, HsSLC22A16 as set forth in SEQ ID NO: 41 or HsSLC22A32 as set forth in SEQ ID NO: 42 or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In specific embodiments, the yeast cell expresses an an ergothioneine transporter such as AtOCT as set forth in SEQ ID NO:37, ScAQR1 as set forth in SEQ ID NO:39, HsSLC22A16 as set forth in SEQ ID NO: 41 or HsSLC22A32 as set forth in SEQ ID NO: 43 or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cell expresses an ergothioneine transporter such as AtOCT as set forth in SEQ ID NO:37, ScAQR1 as set forth in SEQ ID NO:39, HsSLC22A16 as set forth in SEQ ID NO: 41 or HsSLC22A32 as set forth in SEQ ID NO: 43 or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes which are not:
iii) NcEgt1 and NcEgt2; or
viii) SpEgt1 and MsEgtE; or
x) CpEgt1 and SpEgt2.

In specific embodiments, the yeast cell expresses an ergothioneine transporter such as AtOCT1 as set forth in SEQ ID NO:37, ScAQR1 as set forth in SEQ ID NO:39,

HsSLC22A16 as set forth in SEQ ID NO: 41 or HsSLC22A32 as set forth in SEQ ID NO: 43 or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses two copies of NcEgt1 and two copies of CpEgt2.

In some embodiments, the yeast cell carries a deletion of a gene encoding an ergothioneine transporter of S. cerevisiae such as ScAGP2 (GenBank Accession no. JRIV01000019.1), ScTPO3 (GenBank Accession no. BK006949.2), ScTPO4 (GenBank Accession no. JRIV01000150.1), and/or ScTPO1 (GenBank Accession no. JRIV01000165.1) or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. A functional variant here refers to variants such as mutants which retain total or partial ergothioneine transporter activity. The skilled person knows how to determine whether a functional variant retains said activity.

In some embodiments, the yeast cell carries a deletion of a gene encoding an ergothioneine transporter of S. cerevisiae such as ScAGP2 (GenBank Accession no. JRIV01000019.1), ScTPO3 (GenBank Accession no. BK006949.2), ScTPO4 (GenBank Accession no. JRIV01000150.1), and/or ScTPO1 (GenBank Accession no. JRIV01000165.1) or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In specific embodiments, the yeast cell carries a deletion of a gene encoding an ergothioneine transporter of S. cerevisiae such as ScAGP2 (GenBank Accession no. JRIV01000019.1), ScTPO3 (GenBank Accession no. BK006949.2), ScTPO4 (GenBank Accession no. JRIV01000150.1), and/or ScTPO1 (GenBank Accession no. JRIV01000165.1) or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cell carries a deletion of a gene encoding an ergothioneine transporter of S. cerevisiae such as ScAGP2 (GenBank Accession no. JRIV01000019.1), ScTPO3 (GenBank Accession no. BK006949.2), ScTPO4 (GenBank Accession no. JRIV01000150.1), and/or ScTPO1 (GenBank Accession no. JRIV01000165.1) or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes which are not:
iii) NcEgt1 and NcEgt2; or
viii) SpEgt1 and MsEgtE; or
x) CpEgt1 and SpEgt2.

In specific embodiments, the yeast cell carries a deletion of a gene encoding an ergothioneine transporter of S. cerevisiae such as ScAGP2 (GenBank Accession no. JRIV01000019.1), ScTPO3 (GenBank Accession no. BK006949.2), ScTPO4 (GenBank Accession no. JRIV01000150.1), and/or ScTPO1 (GenBank Accession no. JRIV01000165.1) or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses two copies of NcEgt1 and two copies of CpEgt2.

The yeast cell may have one or more of the genotypes described above, such as any of the combinations of the expression of the genes or deletions of the genes as described herein above.

In one embodiment, the yeast cell according to the invention further expresses MsErgt. In addition to expressing MsErgt said yeast cell may also express one or more, two or more, three or more, or four or more or five or more of the genes HsSLC22A4, AtOCT1, ScAQR1, HsSLC22A16 and HsSLC22A32 and/or carry one or more, two or more, three or more or four or more deletions of the genes ScAGP2, ScTPO4, ScTPO3 and ScTPO1.

In one embodiment, the yeast cell according to the invention further expresses HsSLC22A4. In addition to expressing HsSLC22A4 said yeast cell may also express one or more, two or more, three or more or four or more of the genes AtOCT1, ScAQR1, HsSLC22A16 and HsSLC22A32 and/or carry one or more, two or more, three or more or four or more deletions of the genes ScAGP2, ScTPO4, ScTPO3 and ScTPO1.

In one embodiment, the yeast cell according to the invention further expresses HsSLC22A4. In addition to expressing HsSLC22A4 said yeast cell may also express one or more, two or more, three or more, or four or more of the genes AtOCT1, ScAQR1, HsSLC22A16 and HsSLC22A32 and/or carry one or more, two or more, three or more or four or more deletions of the genes ScAGP2, ScTPO4, ScTPO3 and ScTPO1.

In one embodiment, the yeast cell according to the invention further expresses AtOCT1. In addition to expressing AtOCT1 said yeast cell may also express one or more, two or more, three or more of the genes ScAQR1, HsSLC22A16 and HsSLC22A32 and/or carry one or more, two or more, three or more or four or more deletions of the genes ScAGP2, ScTPO4, ScTPO3 and ScTPO1.

In one embodiment, the yeast cell according to the invention further expresses HsSLC22A16. In addition to expressing HsSLC22A16 said yeast cell may also express one or more or two or more of the genes HsSLC22A16 and HsSLC22A32 and/or carry one or more, two or more, three or more or four or more deletions of the genes ScAGP2, ScTPO4, ScTPO3 and ScTPO1.

In one embodiment, the yeast cell according to the invention further expresses HsSLC22A32. In addition to expressing HsSLC22A32 said yeast cell may also express HsSLC22A32 and/or carry one or more, two or more, three or more or four or more deletions of the genes ScAGP2, ScTPO4, ScTPO3 and ScTPO1.

In one embodiment, the yeast cell according to the invention further carries a deletion of ScAGP2. In addition to carrying a deletion of ScAGP2 said yeast cell may also carry one or more, two or more, three or more deletions of the genes ScTPO4, ScTPO3 and ScTPO1.

In one embodiment, the yeast cell according to the invention further carries a deletion of ScTPO4. In addition to carrying a deletion of ScTPO4 said yeast cell may also carry one or more, two or more deletions of the genes ScTPO3 and ScTPO1.

In one embodiment, the yeast cell according to the invention further carries a deletion of ScTPO3. In addition to carrying a deletion of ScTPO3 said yeast cell may also carry a deletion of ScTPO1.

Ergothioneine Titers

The yeast cells disclosed herein are capable of producing ergothioneine with a total titer of at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L, such as at least 11 mg/L, such as at least 12 mg/L, such as at least 13 mg/L, such as at least 14 mg/L, such as at least 15 mg/L, such as at least 20 mg/L, such as at least 25 mg/L, such as at least 30 mg/L, such as at least 35 mg/L, such as at least 40 mg/L, such as at least 45 mg/L, such as at least 50 mg/L, such as at least 100 mg/L, such as at least 150 mg/L, such as at least 200 mg/L, such as at least 300 mg/L, such as at least 400 mg/L, such as at least 500 mg/L, such as at least 600 mg/L, such as at least 700 mg/L, such as at least 800 mg/L, such as at least 900 mg/L, such as at least 1 g/L, or more, wherein the total titer is the sum of the intracellular ergothioneine titer and the extracellular ergothioneine titer. Indeed, the produced ergothioneine may be secreted from the cell—extracellular ergothioneine—or it may be retained in the cell—intracellular ergothioneine.

The yeast cell may be capable of producing extracellular ergothioneine with a titer of at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L, such as at least 11 mg/L, such as at least 12 mg/L, such as at least 13 mg/L, such as at least 14 mg/L, such as at least 15 mg/L, such as at least 20 mg/L, such as at least 25 mg/L, such as at least 30 mg/L, such as at least 35 mg/L, such as at least 40 mg/L, such as at least 45 mg/L, such as at least 50 mg/L, such as at least 100 mg/L, such as at least 150 mg/L, such as at least 200 mg/L, such as at least 300 mg/L, such as at least 400 mg/L, such as at least 500 mg/L, such as at least 600 mg/L, such as at least 700 mg/L, such as at least 800 mg/L, such as at least 900 mg/L, such as at least 1 g/L, or more.

The yeast cell may be capable of producing intracellular ergothioneine with a titer of at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L, such as at least 11 mg/L, such as at least 12 mg/L, such as at least 13 mg/L, such as at least 14 mg/L, such as at least 15 mg/L, such as at least 20 mg/L, such as at least 25 mg/L, such as at least 30 mg/L, such as at least 35 mg/L, such as at least 40 mg/L, such as at least 45 mg/L, such as at least 50 mg/L, such as at least 100 mg/L, such as at least 150 mg/L, such as at least 200 mg/L, such as at least 300 mg/L, such as at least 400 mg/L, such as at least 500 mg/L, such as at least 600 mg/L, such as at least 700 mg/L, such as at least 800 mg/L, such as at least 900 mg/L, such as at least 1 g/L, or more.

Methods for determining the ergothioneine titer are known in the art. For example, the cells can be lysed and the titers determined by HPLC (see example 1) to determine the intracellular ergothioneine titers. The titers can also be determined by HPLC in supernatant fractions from which the cells have been removed.

In one embodiment, the yeast cell according to the present invention is Y. lipolytica may be capable of producing ergothioneine with a titer of at least 100 mg/L, such as at least 150 mg/L, such as at least 200 mg/L, such as at least 250 mg/L, such as at least 260 mg/L, such as at least 270 mg/L ergothioneine.

Other Modifications

The yeast cell according to the present invention is capable of producing ergothioneine, said yeast cell expresses at least one first heterologous enzyme and at least one second heterologous enzyme as described herein above. In some embodiments, the yeast cell according to the present invention expresses at least two copies of the gene encoding the first heterologous enzymes and at least two copies of the gene encoding the second heterologous enzymes.

It is generally contemplated that a yeast cell carrying at least two or more copies of the same gene, such as at least three or more copies, such as at least four or more copies, such as at least four or more copies of the same gene, is capable of producing a higher amount of the protein which the gene encodes, compared to the amount of the same protein produced by a yeast cell carrying only one copy of said gene.

In some embodiments of the present invention, the yeast cell may further comprise one or more additional modifications, such as:
carrying one or more mutations in one or more genes, such as a deletion of a gene; and/or
carrying at least one or more additional copies of one or more genes, in other words expressing and/or overexpressing at least one or more additional genes.

The term “mutations” as used herein include insertions, deletions, substitutions, transversions, and point mutations in the coding and noncoding regions of a gene. Point mutations may concern changes of one base pair, and may result in premature stop codons, frameshift mutations, mutation of a splice site or amino acid substitutions. A mutation as described herein may be a mutation resulting in a linking of two proteins. A gene comprising a mutation may be referred to as a “mutant gene”. If said mutant gene encodes a polypeptide with a sequence different to the wild type, said polypeptide may be referred to as a “mutant polypeptide” and/or “mutant protein”. A mutant polypeptide may be described as carrying a mutation, when it comprises an amino acid sequence differing from the wild type sequence.

The specific genes identified in S. cerevisiae, as described herein, encodes specific proteins. In other yeast species, the specific gene may be differently annotated, but however still encode a similar protein or a functional homologue sharing a similar function. Thus, the knowledge from S. cerevisiae can be transferred to other species, such as other yeast species, e.g. Y. lipolytica. The skilled person will know how to identify the corresponding proteins or genes to be modified, mutated, deleted or overexpressed, based on the information provided herein for S. cerevisiae.

Without being bound by theory, it may be advantageous to modify the following pathways in the yeast cell:
Increase the availability of nitrogen for the ergothioneine precursors S-adenosylmethionine (SAM), histidine and cysteine by nitrogen catabolite repression and/or Transport of nitrogenous compounds
General amino acid control to improve all synthesis of all ergothioneine precursors
Individual amino acid biosynthesis pathways, such as S-adenosylmethionine (SAM), histidine, cysteine and arginine
Sulfur assimilation pathway

Hereby modifying the yeast cell in such a manner that ergothioneine metabolism is directed towards increased ergothioneine synthesis, thereby further increasing the titers of ergothioneine.

Increased Nitrogen Availability for Ergothioneine Precursors

In some embodiments, the yeast cell is capable of increasing the availability of nitrogen for S-adenosylmethionine (SAM), histidine and cysteine. The yeast cell may natively be able to do so, or it may be further modified to improve availability of nitrogen for the precursors S-adenosylmethionine (SAM), histidine and cysteine. This can be done by targeting nitrogen catabolite repression and/or transport of nitrogen.

In one embodiment, the yeast cell carries one or more mutations resulting in decreased nitrogen catabolite repression. In other words, the yeast cell further comprises one or more mutations resulting in increased availability of S-adenosylmethionine (SAM), histidine and cysteine.

In specific embodiments, decreased nitrogen catabolite repression can be done by derepression of nitrogen catabolite repression controlled genes, such as transcriptional regulators. One non-limiting example hereof is deletion or inactivation of nitrogen catabolite repression transcriptional regulator genes, resulting in total or partial loss of function of the corresponding protein. For example the transcriptional activator-encoding gene ScURE2 (GenBank Accession no. JRIV01000061.1) may be mutated or deleted in Saccharomyces cerevisiae. Thus, in one embodiment, the yeast cell carries one or more mutation(s) in the ScURE2 gene.

In some embodiments, the yeast cell carries a deletion of a gene encoding a transcriptional regulator of nitrogen catabolite repression, such as ScURE2 (GenBank Accession no. JRIV01000061.1) or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,
or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In one embodiment, the yeast cell is S. cerevisiae, carries a deletion or mutation of ScURE2, and expresses two copies of NcEgt1 and two copies of CpEgt2.

In another embodiment, the yeast cell is Y. lipolytica, carries a mutation resulting in reduced activity of Ure2 or carries a mutation resulting in reduced activity of a at least one protein having at least 70% sequence homology to Ure2.

Improved availability of nitrogen can also be done by expression or overexpression of genes regulating nitrogen-responsive genes, thus resulting in derepression of nitrogen catabolite repression. In S. cerevisiae, an example of such a gene is ScARG82 (GenBank Accession no. JRIV01000074.1) Thus, in one embodiment, the yeast cell, preferably S. cerevisiae, further expresses or overexpresses ScARG82.

In some embodiments, the yeast cell further expresses or overexpresses ScARG82. In one embodiment, the yeast cell carries at least one additional copy of ScARG82, such as at least two additional copies, such as at least three additional copies, such as at least four additional copies of ScARG82 or a functional homologue thereof having at least 70% homology thereto, such as at least 75%, such as at least 80%, such as at least 85%, such as at least 90%, such as at least 95% homology thereto. In one embodiment, the yeast cell is capable of reducing the transport of basic amino acids, such as histidine and/or SAM to vacuoles. The yeast cell may natively be able to do so, or it may be further modified to reduce the transport of a basic amino acid, in particular histidine, and/or SAM to vacuoles. This can be done by introducing one or more mutation(s) in one or more genes resulting in decreased transport of histidine and/or SAM to vacuoles. In S. cerevisiae examples of such genes are ScVBA1 (GenBank Accession no. JRIV01000175.), ScVBA2 (GenBank Accession no. JRIV01000033.1), and/or ScVBA3 (GenBank Accession no. BK006937.2) or functional homologues thereof sharing at least 70%, such as at least 75%, such as at least 80%, such as at least 85%, such as at least 90%, such as at least 95% homology to ScVBA1 (GenBank Accession no. JRIV01000175.), ScVBA2 (GenBank Accession no. JRIV01000033.1), ScVBA3 (GenBank Accession no. BK006937.2), which encode permeases involved in the transport of basic amino acids, and/or ScPET8 ((GenBank Accession no. JRIV01000154.1) or a functional homolog thereof sharing at least 70%, such as at least 75%, such as at least 80%, such as at least 85%, such as at least 90%, such as at least 95% homology thereto. In one embodiment, the yeast cell is S. cerevisiae, carries a deletion or mutation of the ScVBA2 gene. In one embodiment, the yeast cell is S. cerevisiae, carries a deletion or mutation of the ScVBA1 gene. In one embodiment, the yeast cell is S. cerevisiae, carries a deletion or mutation of the ScVBA3 gene. In one embodiment, the yeast cell is S. cerevisiae, carries a deletion or mutation of the ScPET8 gene.

In another embodiment, the yeast cell is capable of increasing nitrogen transport into the cell. The yeast cell may natively be able to do so, or it may be further modified to improve nitrogen transport into the cell. This can also be done by expression or overexpression of genes increasing nitrogen transport into the cell, such as expression or overexpression of ScSSY1 (GenBank Accession no. JRIV01000074.1), ScGRR1 (GenBank Accession no. JRIV01000227.1), ScYCK2 (GenBank Accession no. JRIV01000213.1), ScSTP1 (GenBank Accession no. JRIV01000080.1), ScSSY5 (GenBank Accession no. JRIV01000167.1), ScPTR3 (GenBank Accession no. JRIV01000088.1) and/or ScSTP2 (GenBank Accession no. JRIV01000156.1) or functional homologues thereof sharing at least 70%, such as at least 75%, such as at least 80%, such as at least 85%, such as at least 90%, such as at least 95% homology to ScSSY1 (GenBank Accession no. JRIV01000074.1), ScGRR1 (GenBank Accession no. JRIV01000227.1), ScYCK2 (GenBank Accession no. JRIV01000213.1), ScSTP1 (GenBank Accession no. JRIV01000080.1), ScSSY5 (GenBank Accession no. JRIV01000167.1), ScPTR3 (GenBank Accession no. JRIV01000088.1) and/or ScSTP2 (GenBank Accession no. JRIV01000156.1).

In one embodiment, the yeast cell further expresses or overexpresses ScSSY1. In one embodiment, the yeast cell further expresses or overexpresses ScGRR1. In one embodiment, the yeast cell further expresses or overexpresses ScYCK2. In one embodiment, the yeast cell further expresses or overexpresses ScSSY5. In one embodiment, the yeast cell further expresses or overexpresses ScPTR3. In one embodiment, the yeast cell further expresses or overexpresses ScSTP2.

In some embodiments, the yeast cell further expresses or overexpresses ScSSY1 or a functional homologue thereof having at least 70% homology thereto. In one embodiment, the yeast cell carries at least one additional copy of ScSSY1, such as at least two additional copies, such as at least three additional copies, such as at least four additional copies of ScSSY1.

In some embodiments, the yeast cell further expresses or overexpresses ScGRR1 or a functional homologue thereof having at least 70% homology thereto. In one embodiment, the yeast cell carries at least one additional copy of ScGRR1, such as at least two additional copies, such as at least three additional copies, such as at least four additional copies of ScGRR1.

In some embodiments, the yeast cell further expresses or overexpresses ScYCK2 or a functional homologue thereof having at least 70% homology thereto. In one embodiment, the yeast cell carries at least one additional copy of ScYCK2, such as at least two additional copies, such as at least three additional copies, such as at least four additional copies of ScYCK2.

In some embodiments, the yeast cell further expresses or overexpresses ScSSY5 or a functional homologue thereof having at least 70% homology thereto. In one embodiment, the yeast cell carries at least one additional copy of ScSSY1, such as at least two additional copies, such as at least three additional copies, such as at least four additional copies of ScSSY1.

In some embodiments, the yeast cell further expresses or overexpresses ScPTR3 or a functional homologue thereof having at least 70% homology thereto. In one embodiment, the yeast cell carries at least one additional copy of ScSSY1, such as at least two additional copies, such as at least three additional copies, such as at least four additional copies of ScSSY1.

In some embodiments, the yeast cell further expresses or overexpresses ScSTP1 or a functional homologue thereof having at least 70% homology thereto. In one embodiment, the yeast cell carries at least one additional copy of ScSSY1, such as at least two additional copies, such as at least three additional copies, such as at least four additional copies of ScSSY1.

In some embodiments, the yeast cell further expresses or overexpresses ScSTP1 or a functional homologue thereof having at least 70% homology thereto. In one embodiment, the yeast cell carries at least one additional copy of ScSTP1, such as at least two additional copies, such as at least three additional copies, such as at least four additional copies of ScSTP1.

In one embodiment, the yeast cell further expresses or overexpresses ScSTP1 as set forth in SEQ ID NO: 45 or sequence having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cell expresses or overexpresses a transcription factor of nitrogenous compound transporters, such as ScSTP1 as set forth in SED ID NO: 45 or functional homologue having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,
or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In one embodiment, the yeast cell expresses or overexpresses ScSTP1 as set forth in SED ID NO: 45 or a sequence having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity thereto, and two copies of NcEgt1 and two copies of CpEgt2.

The gene encoding ScSTP1 is set forth in SEQ ID NO: 46.

In another embodiment, the yeast cell is Y. lipolytica, carries a mutation resulting in reduced activity of Stp1 or carries a mutation resulting in reduced activity of a at least one protein having at least 70% sequence homology to Stp1.

General Amino Acid Control and Individual Amino Acid Biosynthesis Pathways

In some embodiments, the yeast cell is capable of increasing amino acid biosynthesis, especially the biosynthesis of ergothioneine precursors S-adenosylmethionine (SAM), histidine and cysteine. The yeast cell may natively be able to do so, or it may be further modified to improve amino acids biosynthesis. This can be done by modification of the general amino acid control and/or modifications of individual amino acid biosynthesis pathways. In one embodiment, the yeast cell further carries one or more mutation(s) in one or more gene(s) resulting in increased amino acid biosynthesis. In some embodiments, the yeast cell carries one or more mutation(s) in one or more gene(s) resulting in increased arginine, histidine, cysteine and/or S-adenosylmethionine biosynthesis.

In specific embodiments, increased amino acid biosynthesis can be done by derepression of amino acid biosynthesis genes, such as increased and/or constitutive activation of ScGCN2 (GenBank Accession no. JRIV01000117.1) and/or ScGCN4 (GenBank Accession no. JRIV01000017.1). In one embodiment, the yeast cell carries one or more mutation(s) improving amino acid biosynthesis. In one embodiment, the yeast cell carries a mutation in the ScGCN2 gene, resulting in increased activity of Gcn2. In another embodiment, the yeast cell is S. cerevisiae, carries a deletion of the leader sequence in front of ScGCN4. In another embodiment, the yeast cell is S. cerevisiae, carries a deletion of the upstream start codons of ScGCN4. It is generally known that, in front of the ORF of GCN4 there are four start codons that lead to an inactive GCN4 due to premature stop codons. The cell regulates by transcription of GCN4 by blocking/unblocking of these upstream start codons. Constitutively activation of GCN4 may be achieved by deleting the upstream start codons and/or by deleting the leader sequence in front of GCN4 containing the upstream start codons. In another embodiment, the yeast cell carries a mutation in the ScPET18 gene.

In some embodiments, the yeast cell carries one or more mutation(s) in one or more upstream start codons and/or leader sequence of ScGCN4, or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,
or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In one embodiment, the yeast cell, preferably S. cerevisiae, carries one or more mutation(s) in one or more upstream start codons and/or leader sequence of ScGCN4, and expresses two copies of NcEgt1 and two copies of CpEgt2.

Improved biosynthesis of amino acids can also be done by upregulating arginine biosynthesis. In one embodiment, the yeast cell is S. cerevisiae, carries a mutation in ScARG81, such as a deletion or mutation of ScARG81.

Improved biosynthesis of amino acids can also be done by upregulating histidine biosynthesis. In one embodiment, the yeast cell carries one or more mutation(s) in genes improving histidine biosynthesis. In one embodiment, the yeast cell carries one or more mutation(s) in ScBAS1 (GenBank Accession no. JRIV01000108.1) and/or ScPHO2 (GenBank Accession no. JRIV01000173.1) or a functional homologue thereof having at least 70% homology to ScBAS1 and/or ScPHO2, resulting in linked or fused Bas1 and Pho2 proteins. Linking of Bas1 and Pho2 may be achieved as described in Pinson et al. 2000. Thus, a chimera between Bas1 and Pho2 can be performed by connecting the ScBAS1 gene and the ScPHO2 gene with the BAS1 promoter.

In one embodiment, the yeast cell carries a fused ScBAS1 gene and ScPHO2 gene as set forth in SEQ ID NO: 51 or a functional homologue thereof, such as at least 70%, such as at least 75%, such as at least 80%,k such as at least 85% homology thereto.

In some embodiments, the yeast cell carries one or more mutation(s) in one or more gene(s) encoding histidine, such as ScHIS1 (GenBank accession no. JRIV01000173.1).

Thus, in one embodiment, the mutation in HIS1 is one of the following mutations:
a. a mutation resulting in a frameshift mutation;
b. a mutation resulting in formation of a premature stop codon in the ScHIS1 gene;
c. a mutation in a splice site of the ScHIS1 gene;
d. a mutation in the promoter region of the ScHIS1 gene; and/or
e. a mutation in an intron of the ScHIS1 gene.

In one embodiment, the yeast cell according to the present invention is capable of producing at least 100 mg/L, such as at least 150 mg/L, such as at least 200 mg/L, such as at least 250 mg/L histidine.

Improved biosynthesis of amino acids can also be done by upregulating cysteine biosynthesis. In one embodiment, the yeast cell carries one or more mutation(s) in one or more gene(s) improving cysteine biosynthesis. In one embodiment, the yeast cell carries one or more mutation(s) resulting in increased synthesis of cysteine from homocysteine. In one embodiment, the yeast cell further expresses ScCYS3 (GenBank Accession no. JRIV01000001.1) or a functional homologue thereof having at least 70%, such as at least 75%, such as at least 80% such as at least 85% such as at least 90% such as at least 95% homology thereto. In one embodiment, the yeast cell carries at least one additional copy of ScCYS3, such as at least two additional copies, such as at least three additional copies, such as at least four additional copies of ScCYS3. In one embodiment, the yeast cell further expresses ScCYS4 (GenBank Accession no. JRIV01000163.1) or a functional homologue thereof having at least 70%, such as at least 75%, such as at least 80% such as at least 85% such as at least 90% such as at least 95% homology thereto. In one embodiment, the yeast cell carries an additional copy of ScCYS4, such as at least two additional copies, such as at least three additional copies, such as at least four additional copies of ScCYS4. In another embodiment, the yeast cell carries one or more mutation(s) resulting in decreased conversion of cysteine towards homocysteine. In one embodiment, the yeast cell is S. cerevisiae, carries a mutation in or a deletion of ScSTR2 (GenBank Accession no. JRIV01000227.1) or a functional homologue thereof having at least 70%, such as at least 75%, such as at least 80% such as at least 85% such as at least 90% such as at least 95% homology thereto. In one embodiment, the yeast cell carries a mutation in ScSTR3, such as a deletion of or mutation in ScSTR3 (GenBank Accession no. JRIV01000013.1) or a functional homologue thereof having at least 70%, such as at least 75%, such as at least 80% such as at least 85% such as at least 90% such as at least 95% homology thereto. In one embodiment, the yeast cell is S. cerevisiae, carries a mutation in ScGSH1, such as a deletion or mutation of ScGSH1 (GenBank Accession no. JRIV01000144.1).

In some embodiments, the yeast cell, preferably S. cerevisiae, carries a deletion or mutation of a gene encoding a cystathionine gamma-synthase of cysteine biosynthesis, such as ScSTR2, or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,
or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In one embodiment, the yeast cell is S. cerevisiae, carries a deletion or mutation of ScSTR2, and expresses two copies of NcEgt1 and two copies of CpEgt2.

In another embodiment, the yeast cell is Y. lipolytica, carries a mutation resulting in reduced activity of Str2 or carries a mutation resulting in reduced activity of a at least one protein having at least 70% sequence homology to Str2.

In some embodiments, the yeast cell carries one or more mutation(s) in a gene encoding an ATP phosphoribosyltransferase of histidine biosynthesis, such as ScHIS1, or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In one embodiment, the yeast cell carries one or more mutation(s) in HIS1, and expresses two copies of NcEgt1 and two copies of CpEgt2.

In another embodiment embodiments, the yeast cell is capable of producing at least 100 mg/L, such as at least 150 mg/L, such as at least 200 mg/L, such as at least 250 mg/L histidine, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In one embodiment, the yeast cell is capable of producing at least 100 mg/L, such as at least 150 mg/L, such as at least 200 mg/L, such as at least 250 mg/L histidine, and expresses two copies of NcEgt1 and two copies of CpEgt2.

An yeast cell capable of increase histidine production can be achieved as is known in the art, for example by growing the yeast cell in the presence of β-(1,2,4-triazol-3-yl)-DL-alanine. To survive, the yeast cells start overproducing histidine by removing feedback inhibition on the pathway and the cells are then resistant to β-(1,2,4-triazol-3-yl)-DL-alanine (TRA^{R}) and overproduce histidine. See Example 13 as described herein below for production of TRA^{R} yeast cells.

Improved biosynthesis of amino acids can also be done by upregulating S-adenosylmethionine (SAM) biosynthesis. In one embodiment, the yeast cell carries one or more mutation(s) in genes improving S-adenosylmethionine (SAM) biosynthesis. In one embodiment, the yeast cell carries one or more mutation(s) resulting in increased S-adenosylmethionine (SAM) production and/or pool. In one embodiment, the yeast cell further expresses ScSAM2. In one embodiment, the yeast cell carries an additional copy of ScSAM2 (GenBank Accession no. JRIV01000080.1) or a functional homologue thereof having at least 70% homology thereto, such as at least 75%, such as at least 80%, such as at least 85%, such as at least 90%, such as at least 95%, homology thereto. In one embodiment, the yeast cell is S. cerevisiae, carries a mutation in or a deletion of ScGLC3 (GenBank Accession no. BK006939.2) or a functional homologue thereof having at least 70% homology thereto, such as at least 75%, such as at least 80%, such as at least 85%, such as at least 90%, such as at least 95%, homology thereto. In one embodiment, the yeast cell is S. cerevisiae, carries a mutation in or a deletion of ScSPE2 (GenBank Accession no. JRIV01000055.1) or a functional homologue thereof having at least 70% homology thereto, such as at least 75%, such as at least 80%, such as at least 85%, such as at least 90%, such as at least 95%, homology thereto. In one embodiment, the yeast cell carries is S. cerevisiae a mutation in or deletion of ScERG4 (GenBank Accession no. JRIV01000085.1) or a functional homologue thereof having at least 70% homology thereto, such as at least 75%, such as at least 80%, such as at least 85%, such as at least 90%, such as at least 95%, homology thereto. In one embodiment, the yeast cell carries one or more mutation(s) resulting in the removal of feedback resistance of ScMET13 (GenBank Accession no. JRIV01000134.1). In one embodiment, the yeast cell carries a mutation in ScMTHFR.

In some embodiments, the yeast cell is S. cerevisiae, carries a deletion or a mutation of a gene encoding a S-adenosylmethionine decarboxylase of S-adenosylmethionine (SAM) biosynthesis, such as ScSPE2, or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In one embodiment, the yeast cell is S. cerevisiae, carries a deletion or mutation of ScSPE2, and expresses two copies of NcEgt1 and two copies of CpEgt2.

In some embodiments, the yeast cell is S. cerevisiae, carries a deletion or mutation of a gene encoding a delta(24(24(1)))-sterol reductase of S-adenosylmethionine (SAM) biosynthesis, such as ScERG4, or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In one embodiment, the yeast cell is S. cerevisiae, carries a deletion or mutation of ScERG4, and expresses two copies of NcEgt1 and two copies of CpEgt2.

Sulphur Assimilation Pathway

In some embodiments, the yeast cell is capable of improving the sulphur assimilation pathway. The yeast cell may natively be able to do so, or it may be further modified to improve sulphur assimilation. This can be done by expression or overexpression of enzymes improving sulphur assimilation, in particular adenylyl-sulphate kinase and/or phosphoadenosine phosphosulphate reductase.

In one embodiment, the yeast cell further expresses or overexpresses ScMET4 (GenBank Accession no JRIV01000213.1) or a functional homologue thereof having at least 70% homology thereto, such as at least 75%, such as at least 80%, such as at least 85%, such as at least 90%, such as at least 95%, homology thereto. In one embodiment, the yeast cell carries at least one additional copy of ScMET4, such as at least two additional copies, such as at least three additional copies, such as at least four additional copies of ScMET4.

In one embodiment, the yeast cell further expresses or overexpresses ScMET14 (GenBank Accession no. JRIV01000011.1) or a functional homologue thereof having at least 70% homology thereto, such as at least 75%, such as at least 80%, such as at least 85%, such as at least 90%, such as at least 95%, homology thereto. In one embodiment, the yeast cell carries at least one additional copy of ScMET14, such as at least two additional copies, such as at least three additional copies, such as at least four additional copies of ScMET14.

In another embodiment, the yeast cell further expresses the adenylyl-sulphate kinase (ScMET14) as set forth in SEQ ID NO: 47 or functional homologue thereof, such as at least 70% identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

The gene encoding ScMET14 is set forth in SEQ ID NO: 48.

In one embodiment, the yeast cell further expresses or overexpresses ScMET16 (Genbank accession no. JRIV01000176.1) or a functional homologue thereof having at least 70% homology thereto, such as at least 75%, such as at least 80%, such as at least 85%, such as at least 90%, such as at least 95%, homology thereto. In one embodiment, the yeast cell carries at least one additional copy of ScMET16, such as at least three copies, such as at least four copies of ScMET16.

In yet another embodiment, the yeast cell further expresses the phosphoadenosine phosphosulphate reductase (ScMET16) as set forth in SEQ ID NO: 49 or a functional homologue thereto, such as at least 70% identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

The gene encoding ScM ET16 is set forth in SEQ ID NO: 50.

In some embodiments, the yeast cell expresses the adenylyl-sulphate kinase (ScMET14) as set forth in SEQ ID NO: 47 or a functional homologue thereof, such as at least 70% identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In one embodiment, the yeast cell expresses the adenylyl-sulphate kinase (ScMET14) as set forth in SEQ ID NO: 47 or a functional homology thereof, such as at least 70% identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and two copies of NcEgt1 and two copies of CpEgt2.

In some embodiments, the yeast cell expresses the phosphoadenosine phosphosulphate reductase (ScMET16) as set forth in SEQ ID NO: 49 or a functional homologue thereof, such as at least 70% identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In one embodiment, the yeast cell expresses the phosphoadenosine phosphosulfate reductase (ScMET16) as set forth in SEQ ID NO: 49 or a functional homologue thereof, such as at least 70% identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and two copies of NcEgt1 and two copies of CpEgt2.

In one embodiment, the yeast cell according to the invention further carries one or more mutation(s) in ScHIS1. In addition to carrying one or more mutation(s) in ScHIS1 said yeast cell may also express one or more, or three or more of the genes ScSTP1, ScMET14 and ScMET16 and/or carry one or more, two or more, three or more or four or more deletions of the genes ScURE2, ScSTR2, ScSPE2 and ScERG4, and/or one or more mutation(s) in one or more start codons of ScGCN4.

In one embodiment, the yeast cell according to the invention is capable of producing at least 100 mg/L, such as at least 150 mg/L, such as at least 200 mg/L, such as at least 250 mg/L histidine. In addition to being capable of producing at least 100 mg/L, such as at least 150 mg/L, such as at least 200 mg/L, such as at least 250 mg/L histidine said yeast cell may also express one or more, two or more, three or more of the genes ScSTP1, ScMET14 and ScMET16 and/or carry one or more, two or more, three or more or four or more deletions of the genes ScURE2, ScSTR2, ScSPE2 and ScERG4, and/or one or more mutation(s) in one or more start codons of ScGCN4.

In one embodiment, the yeast cell according to the invention further expresses ScSTP1. In addition to expressing ScSTP1 said yeast cell may also express one or more, two or more of the genes ScMET14 and ScMET16 and/or carry one or more, two or more, three or more or four or more deletions of the genes ScURE2, ScSTR2, ScSPE2 and ScERG4, and/or one or more mutation(s) in one or more start codons of ScGCN4.

In one embodiment, the yeast cell according to the invention further expresses ScMET14. In addition to expressing ScMET14 said yeast cell may also express ScMET16 and/or carry one or more, two or more, three or more or four or more deletions of the genes ScURE2, ScSTR2, ScSPE2 and ScERG4, and/or one or more mutation(s) in one or more start codons of ScGCN4.

In one embodiment, the yeast cell according to the invention further expresses ScMET16. In addition to expressing ScMET16 said yeast cell may also carry one or more, two or more, three or more or four or more deletions of the genes ScURE2, ScSTR2, ScSPE2 and ScERG4, and/or one or more mutation(s) in one or more start codons of ScGCN4.

In one embodiment, the yeast cell according to the invention further carries a deletion of ScURE2. In addition to carrying a deletion of ScURE2 said yeast cell may also carry one or more, two or more, three or more deletions of the genes ScSTR2, ScSPE2 and ScERG4, and/or one or more mutation(s) in one or more start codons of ScGCN4.

In one embodiment, the yeast cell according to the invention further carries a deletion of ScSTR2. In addition to carrying a deletion of ScSTR2 said yeast cell may also carry one or more or two or more deletions of the genes ScSPE2 and ScERG4, and/or one or more mutation(s) in one or more start codons of ScGCN4.

In one embodiment, the yeast cell according to the invention further carries a deletion of ScERG4. In addition to carrying a deletion of ScERG4 said yeast cell may also carry one or more mutation(s) in one or more start codons of ScGCN4.

Any of these combinations described herein above may be combined with the modifications described in the section “Ergothioneine transporters”.

Methods for Ergothioneine Production

Also provided herein are methods for producing ergothioneine in a yeast cell, comprising the steps of:
i) providing a yeast cell capable of producing ergothioneine, said yeast cell expressing:
   a) at least one first heterologous enzyme capable of converting L-histidine and/or L-cysteine to S-(hercyn-2-yl)-L-cysteine-S-oxide; and
   b) at least one second heterologous enzyme capable of converting S-(hercyn-2-yl)-L-cysteine-S-oxide to 2-(hydroxysulfanyl)-hercynine;
      wherein the yeast cell is further capable of converting 2-(hydroxysulfanyl)-hercynine to ergothioneine;
ii) incubating said yeast cell in a medium;
   thereby obtaining ergothioneine.

Any of the yeast cells described herein, in particular in the section “Yeast cell”, can be used in such methods. In particular, the yeast cell may express a first heterologous enzyme as described herein, for example in section “First heterologous enzyme” above, and a second heterologous enzyme as described herein, for example in section “Second heterologous enzyme” above. In particular embodiments, the yeast cell expresses the combinations listed under section “Combinations of first and second heterologous enzymes”. Production of ergothioneine using such cells can thus be achieved by incubating the yeast cells disclosed herein in a medium, under conditions allowing the yeast cell to produce ergothioneine.

Suitable media are known to the skilled person. Optimisation of the medium and incubation conditions for optimal ergothioneine production are also envisaged.

The yeast cells, in order to produce ergothioneine, need a suitable substrate. Ergothioneine is produced from L-histidine and/or L-cysteine. The yeast cell may be able to synthesise L-histidine and/or L-cysteine, which it can then use as a substrate. Thus, the medium does not necessarily comprise these amino acids. In some cases however it may be useful to supplement the medium with amino acids, in particular, histidine, preferably L-histidine; cysteine, preferably L-cysteine; or methionine, preferably L-methionine. Without being bound by theory, supplementing the medium with amino acids, particularly the ones previously listed, may increase ergothioneine titers.

In some embodiments, the medium comprises at least one amino acid such as histidine, preferably L-histidine, cysteine, preferably L-cysteine, or methionine, preferably L-methionine, preferably at a concentration of at least 0.1 g/L, such as at least 0.2 g/L, such as at least 0.3 g/L, such as at least 0.4 g/L, such as at least 0.5 g/L, such as at least 0.75 g/L, such as at least 1 g/L, such as at least 2 g/L.

In some embodiments of the present methods, the yeast cell expresses a first heterologous enzyme selected from the group consisting of L-histidine Nα-methyltransferases (EC 2.1.1.44), hercynylcysteine S-oxide synthase (EC 1.14.99.51), glutamate-cysteine ligases (EC 6.3.2.2), γ-glutamyl hercynylcysteine S-oxide synthases (EC 1.14.99.50), and γ-glutamyl hercynylcysteine S-oxide hydrolases (EC 3.5.1.118). In some embodiments, the first heterologous enzyme is an enzyme having an EC number selected from EC 2.1.1.44, EC 1.14.99.51, EC 6.3.2.2, EC 1.14.99.50 and EC 3.5.1.118. In one embodiment, the EC number is 2.1.1.44. In another embodiment, the EC number is EC 1.14.99.51.

In some embodiments, the methods comprise providing a yeast cell expressing a first heterologous enzyme and a second heterologous enzyme, where the first heterologous enzyme is Egt1, derived from a eukaryote such as a fungus, for example a yeast. The yeast cell of the present disclosure may, in addition to the first heterologous enzyme, natively express an enzyme capable of catalysing the same reaction as the first heterologous enzyme, or the yeast cell may be devoid of enzyme capable of catalysing this reaction.

In some embodiments, the first heterologous enzyme is derived from a eukaryote and is classified as EC 2.1.1.44 and/or EC.1.14.99.51.

In some embodiments, the first heterologous enzyme is Egt1 from Neurospora crassa, Claviceps purpurea, Schizosaccharomyces pombe, Rhizopus stolonifera, Aspergillus nidulans, Aspergillus niger, Penicillium roqueforti, Penicillium notatum, Sporobolomyces salmonicolor, Aspergillus oryzae, Aspergillus carbonarius, Neurospora tetrasperma, Agaricus bisporus, Pleurotus ostreatus, Lentinula edodes or Grifola frondosa, or a functional variant thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. The term “functional variant” refers to variants such as mutants, which retain total or partial activity and are still capable of converting L-histidine and/or L-cysteine to S-(hercyn-2-yl)-L-cysteine-S-oxide. The skilled person knows how to determine whether a functional variant retains said activity, for example by using liquid chromatography to detect the products, optionally coupled to mass spectrometry.

In some embodiments, the first heterologous enzyme expressed in the yeast cell provided in the first step of the present methods is derived from Neurospora crassa, Schizosaccharomyces pombe, or Claviceps purpurea. The sequences of the corresponding Egt1 enzymes are set forth in SEQ ID NO: 2 (N. crassa), SEQ ID NO: 4 (S. pombe) and SEQ ID NO: 6 (C. purpurea).

In particular embodiments, the first heterologous enzyme is selected from the group consisting of: NcEgt1 (SEQ ID NO: 2), SpEgt1 (SEQ ID NO: 4) and CpEgt1 (SEQ ID NO: 6), and functional variants thereof having at least 70% homology to SEQ ID NO: 2, SEQ ID NO: 4 or SEQ ID NO: 6, %, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the methods comprise providing a yeast cell which expresses a second heterologous enzyme, which in some embodiments is a β-lyase or a hercynylcysteine sulfoxide lyase (EC 4.4.1.-).

In some embodiments, the second heterologous enzyme expressed in the yeast cell provided in the present methods is Egt2, derived from a eukaryote such as a fungus, for example a yeast. The yeast cell of the present disclosure may, in addition to the first heterologous enzyme, natively express an enzyme capable of catalysing the same reaction as the second heterologous enzyme, or the yeast cell may be devoid of enzyme capable of catalysing this reaction. In some embodiments, the second heterologous enzyme is EgtE, derived from a bacteria.

In some embodiments, the second heterologous enzyme is Egt2 from Neurospora crassa, Claviceps purpurea, Schizosaccharomyces pombe, Rhizopus stolonifera, Aspergillus nidulans, Aspergillus niger, Penicillium roqueforti, Penicillium notatum, Sporobolomyces salmonicolor, Aspergillus oryzae, Aspergillus carbonarius, Neurospora tetrasperma, Agaricus bisporus, Pleurotus ostreatus, Lentinula edodes, Grifola frondosa, Ganoderma lucidum, or Cantharellus cibarius, or a functional variant thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. The term “functional variant” refers to variants such as mutants, which retain total or partial activity and are still capable of converting S-(hercyn-2-yl)-L-cysteine-S-oxide to 2-(hydroxysulfanyl)-hercynine. The skilled person knows how to determine whether a functional variant retains said activity, for instance using liquid chromatography to detect the products, optionally coupled to mass spectrometry.

In other embodiments, the second heterologous enzyme is a bacterial EgtE, such as EgtE from Mycobacterium smegmatis, Nocardia asteroids, Streptomyces albus, Streptomyces fradiae, Streptomyces griseus, Actinoplanes philippinensis, Aspergillus fumigatus, Mycobacterium tuberculosis, Mycobacterium kansasii, Mycobacterium intracellulare, Mycobacterium fortuitum, Mycobacterium ulcerans, Mycobacterium balnei, Mycobacterium leprae, Mycobacterium avium, Mycobacterium bovis, Mycobacterium marinum, Mycobacterium microti, Mycobacterium paratuberculosis, Mycobacterium phlei, Rhodococcus rhodocrous (Mycobacterium rhodocrous), Arthrospira platensis, Arthrospira maxima, Aphanizomenon flos-aquae, Scytonema sp., Oscillatoria sp. and Rhodophyta sp., or a functional variant thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. The term “functional variant” refers to variants such as mutants, which retain total or partial activity and are still capable of converting S-(hercyn-2-yl)-L-cysteine-S-oxide to 2-(hydroxysulfanyl)-hercynine. The skilled person knows how to determine whether a functional variant retains said activity.

In some embodiments of the present methods, the second heterologous enzyme is derived from Neurospora crassa, Schizosaccharomyces pombe, Claviceps purpurea or Mycobacterium smegmatis. The sequences of the corresponding Egt2 or EgtE enzymes are set forth in SEQ ID NO: 8 (N. crassa), SEQ ID NO: 10 (S. pombe), SEQ ID NO: 12 (C. purpurea) and SEQ ID NO: 14 (M. smegmatis).

In particular embodiments the second heterologous enzyme expressed in the yeast cell may be selected from NcEgt2 (SEQ ID NO: 8), SpEgt2 (SEQ ID NO: 10), CpEgt2 (SEQ ID NO: 12), and MsEgtE (SEQ ID NO: 14), and functional variants thereof having at least 70% homology to SEQ ID NO: 8, SEQ ID NO: 10, SEQ ID NO: 12 or SEQ ID NO: 14, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Accordingly, in some embodiments, the method comprises providing a yeast cell expressing a first heterologous enzyme and a second heterologous enzyme, wherein:
the first heterologous enzyme is Egt1 from Neurospora crassa, Claviceps purpurea, Schizosaccharomyces pombe, Rhizopus stolonifera, Aspergillus nidulans, Aspergillus niger, Penicillium roqueforti, Penicillium notatum, Sporobolomyces salmonicolor, Aspergillus oryzae, Aspergillus carbonarius, Neurospora tetrasperma, Agaricus bisporus, Pleurotus ostreatus, Lentinula edodes or Grifola frondosa, or a functional variant thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto; and
the second heterologous enzyme is Egt2 from Neurospora crassa, Claviceps purpurea, Schizosaccharomyces pombe, Rhizopus stolonifera, Aspergillus nidulans, Aspergillus niger, Penicillium roqueforti, Penicillium notatum, Sporobolomyces salmonicolor, Aspergillus oryzae, Aspergillus carbonarius, Neurospora tetrasperma, Agaricus bisporus, Pleurotus ostreatus, Lentinula edodes, Grifola frondosa, Ganoderma lucidum, or Cantharellus cibarius, or the second heterologous enzyme is a bacterial EgtE, such as EgtE from Mycobacterium smegmatis, Nocardia asteroids, Streptomyces albus, Streptomyces fradiae, Streptomyces griseus, Actinoplanes philippinensis, Aspergillus fumigatus, Mycobacterium tuberculosis, Mycobacterium kansasii, Mycobacterium intracellulare, Mycobacterium fortuitum, Mycobacterium ulcerans, Mycobacterium balnei, Mycobacterium leprae, Mycobacterium avium, Mycobacterium bovis, Mycobacterium marinum, Mycobacterium microti, Mycobacterium paratuberculosis, Mycobacterium phlei, Rhodococcus rhodocrous (Mycobacterium rhodocrous), Arthrospira platensis, Arthrospira maxima, Aphanizomenon flos-aquae, Scytonema sp., Oscillatoria sp. and Rhodophyta sp., or a functional variant thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In particular embodiments, the first heterologous enzyme is an enzyme as set forth in SEQ ID NO: 2 (N. crassa), SEQ ID NO: 4 (S. pombe) and SEQ ID NO: 6 (C. purpurea), and the second heterologous enzyme is an enzyme as set forth in SEQ ID NO: 8 (N. crassa), SEQ ID NO: 10 (S. pombe), SEQ ID NO: 12 (C. purpurea) and SEQ ID NO: 14 (M. smegmatis), or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments the first and the second heterologous enzymes are:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2;
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In specific embodiments, the yeast cell expresses a first and second heterologous enzymes as follows:
i) NcEgt1 and NcEgt2;
ii) NcEgt1 and SpEgt2;
iii) NcEgt1 and CpEgt2;
iv) NcEgt1 and MsEgtE;
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cells of the invention express a first and a second heterologous enzymes which are not:
i) NcEgt1 and NcEgt2; or
viii) SpEgt1 and MsEgtE; or
x) CpEgt1 and SpEgt2.

Expression of said enzymes can be achieved as is known in the art, for example by introduction in the yeast cell of nucleic acids encoding the first and second heterologous enzymes, as described herein above in the section “nucleic acids encoding the first and second heterologous enzymes”.

In some embodiments, the yeast cell used in the present methods may further express an ergothioneine transporter such as a heterologous ergothioneine transporter, for example the ergothioneine transporter of M. smegmatis as set forth in SEQ ID NO: 35 (MsErgT) or the ergothioneine transporter of H. sapiens as set forth in SEQ ID NO: 36 (HsSLC22A4) or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the methods thus comprise the steps of providing and incubating a yeast cell expressing an ergothioneine transporter such as MsErgT as set forth in SEQ ID NO: 35 or HsSLC22A4 as set forth in SEQ ID NO: 36 or a functional thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In specific embodiments, the yeast cell used in the present methods expresses an ergothioneine transporter such as MsErgT as set forth in SEQ ID NO: 35 or HsSLC22A4 as set forth in SEQ ID NO: 36 or a functional thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
xii) CpEgt1 and MsEgtE,

or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the yeast cell used in the present methods expresses an ergothioneine transporter such as MsErgT as set forth in SEQ ID NO: 35 or

HsSLC22A4 as set forth in SEQ ID NO: 36 or a functional thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes which are not:
iii) NcEgt1 and NcEgt2; or
viii) SpEgt1 and MsEgtE; or
x) CpEgt1 and SpEgt2.

In some embodiments, the yeast cell used in the present methods may further comprise one or more additional modifications as described herein in the section entitled “Ergothionine transporters” and “Other modifications”, in particular:
Increase the availability of nitrogen for the ergothioneine precursors S-adenosylmethionine (SAM), histidine and cysteine by nitrogen catabolite repression and/or Transport of nitrogenous compounds
General amino acid control to improve all synthesis of all ergothioneine precursors
Individual amino acid biosynthesis pathways, such as S-adenosylmethionine (SAM), histidine, cysteine and arginine
Sulfur assimilation pathway
The yeast cell according to any one of the previous items, wherein the yeast cell is capable of producing at least 100 mg/L, such as at least 150 mg/L, such as at least 200 mg/L, such as at least 250 mg/L histidine.

In some embodiments, the yeast cell used in the present methods further expresses or overexpresses one or more of the following:
a ergothioneine transporter, such as MsErgT (SEQ ID NO:35) or variants thereof having at least 70% homology thereto;
a ergothioneine transporter, such as HsSLC22A4 (SEQ ID NO:36) or variants thereof having at least 70% homology thereto;
a ergothioneine transporter, such as AtOCT1 (SEQ ID NO:37) or variants thereof having at least 70% homology thereto;
a ergothioneine transporter, such as ScAQR1 (SEQ ID NO:39) or variants thereof having at least 70% homology thereto;
a ergothioneine transporter, such as HsSLC22A16 (SEQ ID NO:41) or variants thereof having at least 70% homology thereto;
a ergothioneine transporter, such as HsSLC22A32 (SEQ ID NO:43) or variants thereof having at least 70% homology thereto;
an adenylyl-sulfate kinase, such as ScMET14 (SEQ ID NO: 47) or variants thereof having at least 70% homology thereto;
a phosphoadenosine phosphosulfate reductase, such as ScMET16 (SEQ ID NO: 49) or variants thereof having at least 70% homology thereto; and/or
a transcription factor for nitrogenous compound transporters, such as STP1 (SEQ ID NO: 45) or variants thereof having at least 70% homology thereto.

In some embodiments, the yeast cell used in the present methods further comprises one or more mutation(s) in one or more of the following gene(s)
ScAGP2;
ScTPO4;
ScTPO3;
ScTPO1;
ScURE2;
ScSTR2;
ScERG4;
ScSPE2; and/or
ScGCN4, such as one or more mutation(s) in the upstream start codons upstream of GCN4.

The present methods allow production of ergothioneine with a total titer of at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L, such as at least 11 mg/L, such as at least 12 mg/L, such as at least 13 mg/L, such as at least 14 mg/L, such as at least 15 mg/L, such as at least 20 mg/L, such as at least 25 mg/L, such as at least 30 mg/L, such as at least 35 mg/L, such as at least 40 mg/L, such as at least 45 mg/L, such as at least 50 mg/L, such as at least 100 mg/L, such as at least 150 mg/L, such as at least 200 mg/L, such as at least 300 mg/L, such as at least 400 mg/L, such as at least 500 mg/L, such as at least 600 mg/L, such as at least 700 mg/L, such as at least 800 mg/L, such as at least 900 mg/L, such as at least 1 g/L, such as at least 1.1 g/L, such as at least 1.2 g/L, such as at least 1.3 g/L, such as at least 1.4 g/L, such as at least 1.5 g/L or more, wherein the total titer is the sum of the intracellular ergothioneine titer and the extracellular ergothioneine titer. Indeed, the produced ergothioneine may be secreted from the cell—extracellular ergothioneine—or it may be retained in the cell—intracellular ergothioneine.

In particular, the present methods may result in production of extracellular ergothioneine with a titer of at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L, such as at least 11 mg/L, such as at least 12 mg/L, such as at least 13 mg/L, such as at least 14 mg/L, such as at least 15 mg/L, such as at least 20 mg/L, such as at least 25 mg/L, such as at least 30 mg/L, such as at least 35 mg/L, such as at least 40 mg/L, such as at least 45 mg/L, such as at least 50 mg/L, such as at least 100 mg/L, such as at least 150 mg/L, such as at least 200 mg/L, such as at least 300 mg/L, such as at least 400 mg/L, such as at least 500 mg/L, such as at least 600 mg/L, such as at least 700 mg/L, such as at least 800 mg/L, such as at least 900 mg/L, such as at least 1 g/L, such as at least 1.1 g/L, such as at least 1.2 g/L, such as at least 1.3 g/L, such as at least 1.4 g/L, such as at least 1.5 g/L, or more.

The present methods may result in production of intracellular ergothioneine with a titer of at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L, such as at least 11 mg/L, such as at least 12 mg/L, such as at least 13 mg/L, such as at least 14 mg/L, such as at least 15 mg/L, such as at least 20 mg/L, such as at least 25 mg/L, such as at least 30 mg/L, such as at least 35 mg/L, such as at least 40 mg/L, such as at least 45 mg/L, such as at least 50 mg/L, such as at least 100 mg/L, such as at least 150 mg/L, such as at least 200 mg/L, such as at least 300 mg/L, such as at least 400 mg/L, such as at least 500 mg/L, such as at least 600 mg/L, such as at least 700 mg/L, such as at least 800 mg/L, such as at least 900 mg/L, such as at least 1 g/L, such as at least 1.1 g/L, such as at least 1.2 g/L, such as at least 1.3 g/L, such as at least 1.4 g/L, such as at least 1.5 g/L, or more.

The method may also comprise a step of recovering the produced ergothioneine. This may involve a heating step to precipitate cell material and to release intracellular ergothioneine, a centrifugation or filtration step to remove the cell debris and precipitated materials, pH-adjusting and chromatographic steps optionally involving solvents to vary the solubility of the ergothioneine and to purify it from other components. In some embodiments the recovered ergohioneine may be used as a nutritional supplement with its naïve or processed host cells directly.

Polypeptides

The present inventors have identified several polypeptides useful for engineering yeast cells which can produce ergothioneine. In particular, Egt1 and Egt2 from Claviceps purpurea have been identified and found useful for heterologous expression in yeast cells, thereby providing a microbial platform for ergothioneine production.

In particular, herein is provided a polypeptide having the sequence as set forth in SEQ ID NO: 6 (CpEgt1) or a functional variant thereof having at least 70% homology to SEQ ID NO: 6, homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Also provided is a polypeptide having the sequence as set forth in SEQ ID NO: 12 (CpEgt2) or a functional variant thereof having at least 70% homology to SEQ ID NO:

12, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

Also provided are host cells expressing said polypeptides.

Also provided is the use of above polypeptides or host cells for the production of ergothioneine.

Nucleic Acids, Vectors and Host Cells

Also provided herein are nucleic acids encoding the above polypeptides, namely Egt1 and Egt2 from Claviceps purpurea. Such nucleic acids may have been codon-optimised for expression in a yeast cell as is known in the art.

In one embodiment, the nucleic acid has the sequence as set forth in SEQ ID NO: 5 or SEQ ID NO: 16, or has at least 70% homology to SEQ ID NO: 5 or SEQ ID NO: 16, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the nucleic acid has the sequence as set forth in SEQ ID NO: 11 or SEQ ID NO: 18, or has at least 70% homology to SEQ ID NO: 11 or SEQ ID NO: 18, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

The nucleic acids employed for the purpose of the present disclosure may be codon-optimised as is known in the art to improve expression of the proteins they encode in the yeast cell to be modified.

In some embodiments, the nucleic acids encoding the first and the second heterologous enzymes may independently be integrated in the genome of the yeast cell by genome engineering or genome editing or by crossing yeast cells of different mating types, or may be expressed in the cell from a vector.

Methods for integrating a nucleic acid are well known in the art. Thus in some embodiments the first and/or second heterologous enzyme is expressed in the cell by introduction of heterologous nucleic acids encoding them in the yeast cell. The heterologous nucleic acids may be codon-optimised for any purpose, or may comprise features that can help improve the activity. For example, the heterologous nucleic acid may be modified so as to encode a modified protein. Such modifications include, but are not limited to, the introduction of localisation signals, gain-of-function or loss-of-function mutations, fusion of the protein to a marker or a tag such as fluorescent tag, insertion of an inducible promoter, introduction of modifications conferring increased stability and/or half-life.

The introduction of the heterologous nucleic acid encoding the activity of interest can be performed by methods known in the art. The skilled person will recognise that such methods include, but are not limited to: cloning and homologous recombination-based methods. Cloning methods may involve the design and construction of a plasmid in an organism such as Escherichia coli. The plasmid may be an integrative or a non-integrative vector. Cloning-free methods comprise homologous recombination-based methods such as adaptamer-mediated PCR or gap repair. Such methods often result in integration of the heterologous nucleic acid in the genome of the yeast cell.

The nucleic acids may be present in high copy number.

The nucleic acids may be under the control of an inducible promoter, or of a constitutive promoter, as is known in the art. The nucleic acids may be under the control of a strong promoter as is known in the art.

Also provided are vectors comprising the above nucleic acids, as well as host cells comprising said vectors and/or said nucleic acids.

Vectors useful in the context of the present disclosure may comprise:
A nucleic acid encoding a first heterologous enzyme as described herein; and/or
A nucleic acid encoding a second heterologous enzyme as described herein;
And optionally a nucleic acid encoding an ergothioneine transporter as described herein.

Also provided is the use of above nucleic acids, vectors or host cells for the production of ergothioneine.

Also provided is a kit for constructing a yeast cell capable of producing ergothioneine as described herein, wherein the kit comprises:
A yeast cell as described herein and instructions for use;
A parental yeast cell to be modified and nucleic acids or vectors suitable for modifying said yeast cell to obtain a yeast cell as described herein, and instructions for use.

Sequence Overview

| Sequence | | |
|---|---|---|
| ID NO: | Description | Details |
| 1 | NcEgt1 DNA from | Encodes DUF323 domain-containing |
| | Neurospora crassa | protein [Neurospora crassa OR74A] of |
| | | SEQ ID NO: 2 |
| 2 | NcEgt1 protein from | DUF323 domain-containing protein |
| | Neurospora crassa | [Neurospora crassa OR74A] |
| | | NCBI Reference Sequence: |
| | | XP_956324.3 |
| 3 | SpEgt1 DNA from | Encodes sulfatase modifying factor 1-like |
| | Schizosaccharomyces | protein [Schizosaccharomyces pombe] of |
| | pombe | SEQ ID NO: 4 |
| 4 | SpEgt1 protein | sulfatase modifying factor 1-like protein |
| | | [Schizosaccharomyces pombe] |
| | | NCBI Reference Sequence: |
| | | NP_596639.2 |
| 5 | CpEgt1 DNA from | Encodes (Previously) uncharacterized |
| | Claviceps purpura | protein CPUR_07517 [Claviceps |
| | (introns only) | purpurea 20.1] of SEQ ID NO: 6 |
| 6 | CpEgt1 protein from | (Previously) uncharacterized protein |
| | Claviceps purpura | CPUR_07517 [Claviceps purpurea 20.1] |
| | | GenBank: CCE33591.1 |
| 7 | NcEgt2 DNA from | Encodes aminotransferase [Neurospora |
| | Neurospora crassa | crassa OR74A] of SEQ ID NO: 8 |
| 8 | NcEgt2 protein from | aminotransferase [Neurospora crassa |
| | Neurospora crassa | OR74A] |
| | | NCBI Reference Sequence: |
| | | XP_001728131.1 |
| 9 | SpEgt2 DNA from | Encodes putative aminotransferase |
| | Schizosaccharomyces | [Schizosaccharomyces pombe] of SEQ |
| | pombe | ID NO: 10 |
| 10 | SpEgt2 protein from | putative aminotransferase |
| | Schizosaccharomyces | [Schizosaccharomyces pombe] |
| | pombe | NCBI Reference Sequence: |
| | | NP_595091.1 |
| 11 | CpEgt2 DNA from | Encodes protein of SEQ ID NO: 12 |
| | Claviceps purpurea | |
| 12 | CpEgt2 protein from | related to isopenicillin N epimerase |
| | Claviceps purpurea | [Claviceps purpurea 20.1] |
| | | GenBank: CCE33140.1 |
| 13 | MsEgtE DNA from | Encodes pyridoxal-phosphate-dependent |
| | Mycolicibacterium | transferase [Mycolicibacterium |
| | smegmatis MC2 155 | smegmatis MC2 155] of SEQ ID NO: 14 |
| 14 | MsEgtE protein from | pyridoxal-phosphate-dependent |
| | Mycolicibacterium | transferase [Mycolicibacterium |
| | smegmatis MC2 155 | smegmatis MC2 155] |
| 15 | NcEgt1 DNA codon- | |
| | optimised for | |
| | Saccharomyces | |
| | cerevisiae | |
| 16 | CpEgt1 DNA codon- | |
| | optimised for | |
| | Saccharomyces | |
| | cerevisiae | |
| 17 | NcEgt2 DNA codon- | |
| | optimised for | |
| | Saccharomyces | |
| | cerevisiae | |
| 18 | CpEgt2 DNA codon- | |
| | optimised for | |
| | Saccharomyces | |
| | cerevisiae | |
| 19 | MsEgtE DNA codon- | |
| | optimised for | |
| | Saccharomyces | |
| | cerevisiae | |
| 20 | SpEgt1 actual amino | |
| | acid sequence used | |
| 21 | SpEgt2 actual amino | |
| | acid sequence used | |
| 22 | MsEgtA DNA | Encodes Glutamate-cysteine ligase |
| | sequence from | [Mycolicibacterium smegmatis MC2 155] |
| | Mycolicibacterium | of SEQ ID NO: 24 |
| | smegmatis MC2 155 | |
| 23 | MsEgtA DNA codon- | |
| | optimised for S. | |
| | cerevisiae | |
| 24 | MsEgtA protein from | Glutamate-cysteine ligase |
| | Mycolicibacterium | [Mycolicibacterium smegmatis MC2 155] |
| | smegmatis MC2 155 | GenBank: AFP42520.1 |
| 25 | MsEgtB DNA | Encodes ergothioneine biosynthesis |
| | sequence from | protein EgtB [Mycolicibacterium |
| | Mycolicibacterium | smegmatis] of SEQ ID NO: 27 |
| | smegmatis MC2 155 | |
| 26 | MsEgtB DNA codon- | |
| | optimised for S. | |
| | cerevisiae | |
| 27 | MsEgtB protein from | ergothioneine biosynthesis protein EgtB |
| | Mycolicibacterium | [Mycolicibacterium smegmatis] |
| | smegmatis MC2 155 | NCBI Reference Sequence: |
| | | WP_011731158.1 |
| 28 | MsEgtC DNA | Encodes class II glutamine |
| | sequence from | amidotransferase [Mycolicibacterium |
| | Mycolicibacterium | smegmatis] of SEQ ID NO: 30 |
| | smegmatis MC2 155 | |
| 29 | MsEgtC DNA codon- | |
| | optimised for S. | |
| | cerevisiae from | |
| | Mycolicibacterium | |
| | smegmatis MC2 155 | |
| 30 | MsEgtC protein from | class II glutamine amidotransferase |
| | Mycolicibacterium | [Mycolicibacterium smegmatis] |
| | smegmatis MC2 155 | NCBI Reference Sequence: |
| | | WP_011731157.1 |
| 31 | MsEgtD DNA | Encodes L-histidine N(alpha)- |
| | sequence from | methyltransferase [Mycolicibacterium |
| | Mycolicibacterium | smegmatis] of SEQ ID NO: 33 |
| | smegmatis MC2 155 | |
| 32 | MsEgtD DNA codon- | |
| | optimised for S. | |
| | cerevisiae | |
| 33 | MsEgtD protein | L-histidine N(alpha)-methyltransferase |
| | | [Mycolicibacterium smegmatis] |
| | | NCBI Reference Sequence: |
| | | WP_011731156.1 |
| 34 | MsEgtE DNA codon- | |
| | optimised for S. | |
| | cerevisiae | |
| 35 | MsErgt DNA | Encodes putative ergothioneine |
| | sequence from | transporter from M. smegmatis |
| | Mycolicibacterium | |
| | smegmatis | |
| 36 | HsSLC22A4 from | Encodes ergothioneine transporter from |
| | Homo sapiens | Homo sapiens |
| 37 | AtOct1 protein from A. | Organic cation/carnitine transporter 1 |
| | thaliana | |
| 38 | AtOct1 DNA from A. | |
| | thaliana and codon | |
| | optimized for | |
| | Saccharomyces | |
| | cerevisiae | |
| 39 | ScAqr1 protein from | Probable transporter/Multidrug |
| | Saccharomyces | transporter [S. cerevisiae] |
| | cerevisiae | |
| 40 | ScAqr1 DNA from | |
| | Saccharomyces | |
| | cerevisiae | |
| 41 | HsSLC22A16 protein | Solute carrier family 22 member 16 |
| | from Homo sapiens | |
| 42 | HsSLC22A16 DNA | |
| | codon-optimized for | |
| | S. cerevisiae | |
| 43 | HsSLC22A32 protein | Solute carrier family 22 member 32 |
| | from Homo sapiens | |
| 44 | HsSLC22A32 DNA | |
| | codon-optimized for | |
| | S. cerevisiae | |
| 45 | ScSTP1 protein from | Transcription factor |
| | Saccharomyces | |
| | cerevisiae | |
| 46 | ScSTP1 DNA | |
| 47 | ScMET14 protein | Adenylyl-sulfate kinase |
| | from Saccharomyces | |
| | cerevisiae | |
| 48 | ScMET14 DNA | |
| 49 | ScMET16 protein | Phosphoadenosine phosphosulfate |
| | from Saccharomyces | reductase |
| | cerevisiae | |
| 50 | ScMET16 DNA | |
| 51 | BAS1-PHO2 fusion | |
| | DNA from | |
| | Saccharomyces | |
| | cerevisiae | |

### EXAMPLES

### Example 1—Materials and Methods

Strains, Chemicals, Synthetic Genes, Services

In this study, the Saccharomyces cerevisiae strain ST7574 (CEN.PK113-7D strain transformed with a plasmid carrying a Cas9 expression cassette and G418 resistance), was used as the background strain for metabolic engineering. The Yarrowia lipolytica ST6512 (W29 strain with integrated an integrated Cas9 gene and D-serine resistance) was used as the background strain for Y. lipolytica engineering. Escherichia coli DH5a was used for all cloning procedures, propagation and storing of plasmids. Ergothioneine (catalogue #E7521-25MG, ≥98% purity) was bought from Sigma-Aldrich, hercynine (catalogue #H288900, 100 mg, ≥95% purity) was bought from Toronto Research Chemicals Inc. Synthetic genes were ordered through the GeneArt Gene Synthesis service of Thermo Fisher Scientific or the custom gene synthesis service of IDT. Sequencing results were obtained through Eurofins Genomics (Ebersberg, Germany) using their Mix2Seq kit. Enpump 200 was obtained from Enpresso (Berlin, Germany).

Cloning Strategy

All genes necessary from the biosynthesis pathway of ergothioneine were codon-optimized, except for the genes from Schizosaccharomyces pombe, which were isolated from genomic DNA using PCR and appropriate primers. Strain construction for the biosynthesis pathway and subsequent integrations in S. cerevisiae were performed using EasyClone MarkerFree method (Jessop-Fabre et al., 2106). Strain construction for the ergothioneine biosynthesis pathway in Y. lipolytica was performed using EasyCloneYALI method (Holkenbrink et al., 2018). For the deletions in ST9553 through ST9564, the genes were deleted using a kanamycin resistance cassette. Otherwise, deletions were performed using CRISPR/Cas9 methods from Stovicek et al., 2015. Strains were checked for correct integration by colony PCR. A list of the resulting strains can be found in table 1.

TABLE 1

| | | | Parent | Genetic |
|---|---|---|---|---|
| Strain | Characteristics | Strain specifics | strain | edit |
| ST1 | CEN.PK113-7D | Parent strain S. cerevisiae | | |
| | Mata MAL2-8c | | | |
| | SUC2 URA3 HIS3 | | | |
| | LEU2 TRP1 | | | |
| ST4842 | Y. lipolytica W29 | Parent strain Yarrowia | | |
| | MATA | lipolytica | | |
| ST6512 | Y. lipolytica W29 | Background strain for | ST4842 | pCfB6364 |
| | MATA | Yarrowia lipolytica strains | | |
| | ku70Δ::PrTEF1- | | | |
| | Cas9- | | | |
| | TTef12::PrGPD- | | | |
| | DsdA-TLip2 | | | |
| ST7574 | CEN.PK113-7D + | Background strain, | ST1 | pCfB2312 |
| | pCfB2312 (Cas9 | plasmid cured out for ERG | | (no |
| | plasmid) | production experiments | | integration, |
| | | | | episomal) |
| ST8459 | NcEgt1 + | Fungal pathway | ST7574 | pCfB8331, |
| | NcEgt2 | | | pCfB8332 |
| ST8460 | NcEgt1 + | Fungal pathway | ST7574 | pCfB8331, |
| | SpEgt2 | | | pCfB8334 |
| ST8461 | NcEgt1 + | Fungal pathway | ST7574 | pCfB8331, |
| | CpEgt2 | | | pCfB8336 |
| ST8462 | SpEgt1 + | Fungal pathway | ST7574 | pCfB8333, |
| | SpEgt2 | | | pCfB8334 |
| ST8463 | SpEgt1 + | Fungal pathway | ST7574 | pCfB8332, |
| | NcEgt2 | | | pCfB8333 |
| ST8464 | SpEgt1 + | Fungal pathway | ST7574 | pCfB8333, |
| | CpEgt2 | | | pCfB8336 |
| ST8465 | CpEgt1 + | Fungal pathway | ST7574 | pCfB8335, |
| | CpEgt2 | | | pCfB8336 |
| ST8466 | CpEgt1 + | Fungal pathway | ST7574 | pCfB8332, |
| | NcEgt2 | | | pCfB8335 |
| ST8467 | CpEgt1 + | Fungal pathway | ST7574 | pCfB8334, |
| | SpEgt2 | | | pCfB8335 |
| ST8468 | MsEgtD/B + | Bacterial pathway | ST7574 | pCfB8337, |
| | MsEgtA/C + | | | pCfB8338, |
| | MsEgtE | | | pCfB8339 |
| ST8469 | MsEgtD/B + | Mixed pathway | ST7574 | pCfB8332, |
| | MsEgtA/C + | | | pCfB8337, |
| | NcEgt2 | | | pCfB8339 |
| ST8470 | MsEgtD/B + | Mixed pathway | ST7574 | pCfB8334, |
| | MsEgtA/C + | | | pCfB8337, |
| | SpEgt2 | | | pCfB8339 |
| ST8471 | MsEgtD/B + | Mixed pathway | ST7574 | pCfB8336, |
| | MsEgtA/C + | | | pCfB8337, |
| | CpEgt2 | | | pCfB8339 |
| ST8472 | NcEgt1 + | Mixed pathway | ST7574 | pCfB8331, |
| | MsEgtE | | | pCfB8338 |
| ST8473 | SpEgt1 + | Mixed pathway | ST7574 | pCfB8333, |
| | MsEgtE | | | pCfB8338 |
| ST8474 | CpEgt1 + | Mixed pathway | ST7574 | pCfB8335, |
| | MsEgtE | | | pCfB8338 |
| ST8654 | NcEgt1 + | Fungal pathway + putative | ST8461 | pCfB8374 |
| | CpEgt2 + | bacterial transporter from | | |
| | MsMEI_6084 | M. smegmatis | | |
| ST8655 | NcEgt1 + | Fungal pathway + | ST8461 | pCfB8375 |
| | CpEgt2 + | transporter from H. | | |
| | HsSLC22A4X | sapiens | | |
| ST8925 | NcEgt1 + | Fungal pathway with extra | ST8461 | pCfB8805 |
| | CpEgt2 + | CpEgt2 | | |
| | second copy | | | |
| | of CpEgt2 | | | |
| ST8926 | NcEgt1 + | Fungal pathway with extra | ST8461 | pCfB8804 |
| | CpEgt2 + | NcEgt1 | | |
| | second copy | | | |
| | of NcEgt1 | | | |
| ST8927 | NcEgt1 + | Two copies of fungal | ST8461 | pCfB8804, |
| | CpEgt2 + | pathway | | pCfB8805 |
| | second copy | | | |
| | of both NcEgt1 | | | |
| | and CpEgt2 | | | |
| ST9553 | NcEgt1x2 + | Two copies of fungal | ST8927 | BB4174 |
| | CpEgt2x2 + | pathway with URE2 knock- | | |
| | Δure2 | out | | |
| ST9554 | NcEgt1x2 + | Two copies of fungal | ST8927 | BB4175 |
| | CpEgt2x2 + | pathway with VBA1 knock- | | |
| | Δvba1 | out | | |
| ST9555 | NcEgt1x2 + | Two copies of fungal | ST8927 | BB4176 |
| | CpEgt2x2 + | pathway with VBA2 knock- | | |
| | Δvba2 | out | | |
| ST9556 | NcEgt1x2 + | Two copies of fungal | ST8927 | BB4177 |
| | CpEgt2x2 + | pathway with VBA3 knock- | | |
| | Δvba3 | out | | |
| ST9557 | NcEgt1x2 + | Two copies of fungal | ST8927 | BB4178 |
| | CpEgt2x2 + | pathway with ARG81 | | |
| | Δarg81 | knock-out | | |
| ST9558 | NcEgt1x2 + | Two copies of fungal | ST8927 | BB4179 |
| | CpEgt2x2 + | pathway with STR2 knock- | | |
| | Δstr2 | out | | |
| ST9559 | NcEgt1x2 + | Two copies of fungal | ST8927 | BB4180 |
| | CpEgt2x2 + | pathway with GSH1 knock- | | |
| | Δgsh1 | out | | |
| ST9560 | NcEgt1x2 + | Two copies of fungal | ST8927 | BB4181 |
| | CpEgt2x2 + | pathway with URE2 knock- | | |
| | Δglc3 | out | | |
| ST9561 | NcEgt1x2 + | Two copies of fungal | ST8927 | BB4182 |
| | CpEgt2x2 + | pathway with URE2 knock- | | |
| | Δspe2 | out | | |
| ST9562 | NcEgt1x2 + | Two copies of fungal | ST8927 | BB4183 |
| | CpEgt2x2 + | pathway with URE2 knock- | | |
| | Δerg4 | out | | |
| ST9564 | NcEgt1x2 + | Two copies of fungal | ST8927 | BB4185 |
| | CpEgt2x2 + | pathway with URE2 knock- | | |
| | Δpet18 | out | | |
| ST9566 | NcEgt1x2 + | Two copies of fungal | ST8927 | pCfB9198, |
| | CpEgt2x2 + | pathway with GCN4 | | PR-25131 |
| | Δgcn4_uORFS | upstream ORF deletion | | |
| ST9567 | NcEgt1x2 + | Two copies of fungal | ST8927 | pCfB9198, |
| | CpEgt2x2 + | pathway with GCN4 leader | | PR-25132 |
| | Δgcn4_leader | sequence deletion | | |
| ST9569 | NcEgt1x2 + | Two copies of fungal | ST8927 | pCfB9200, |
| | CpEgt2x2 + | pathway with STR3 knock- | | PR-25136 |
| | Δstr3 | out | | |
| ST9570 | NcEgt1x2 + | Two copies of fungal | ST8927 | pCfB9201, |
| | CpEgt2x2 + | pathway with PET8 knock- | | PR-25139 |
| | Δpet8 | out | | |
| ST9571 | NcEgt1x2 + | Two copies of fungal | ST8927 | pCfB9202, |
| | CpEgt2x2 + | pathway with BAS1-PHO2 | | BB4203 |
| | BAS1-PHO2 | fusion | | |
| | fusion | | | |
| ST9572 | NcEgt1x2 + | Two copies of fungal | ST8927 | pCfB9203 |
| | CpEgt2x2 + | pathway with ARG82 | | |
| | ARG82 | integration | | |
| ST9573 | NcEgt1x2 + | Two copies of fungal | ST8927 | pCfB9204 |
| | CpEgt2x2 + | pathway with SSY1 | | |
| | SSY1 | integration | | |
| ST9574 | NcEgt1x2 + | Two copies of fungal | ST8927 | pCfB9205 |
| | CpEgt2x2 + | pathway with GRR1 | | |
| | GRR1 | integration | | |
| ST9575 | NcEgt1x2 + | Two copies of fungal | ST8927 | pCfB9206 |
| | CpEgt2x2 + | pathway with YCK2 | | |
| | YCK2 | integration | | |
| ST9576 | NcEgt1x2 + | Two copies of fungal | ST8927 | pCfB9207 |
| | CpEgt2x2 + | pathway with STP1 | | |
| | STP1 | integration | | |
| ST9577 | NcEgt1x2 + | Two copies of fungal | ST8927 | pCfB9208 |
| | CpEgt2x2 + | pathway with CYS3 | | |
| | CYS3 | integration | | |
| ST9578 | NcEgt1x2 + | Two copies of fungal | ST8927 | pCfB9209 |
| | CpEgt2x2 + | pathway with CYS4 | | |
| | CYS4 | integration | | |
| ST9579 | NcEgt1x2 + | Two copies of fungal | ST8927 | pCfB9210 |
| | CpEgt2x2 + | pathway with SAM2 | | |
| | SAM2 | integration | | |
| ST9580 | NcEgt1x2 + | Two copies of fungal | ST8927 | pCfB9211 |
| | CpEgt2x2 + | pathway with MET4 | | |
| | MET4 | integration | | |
| ST9581 | NcEgt1x2 + | Two copies of fungal | ST8927 | pCfB9212 |
| | CpEgt2x2 + | pathway with MET14 | | |
| | MET14 | integration | | |
| ST9582 | NcEgt1x2 + | Two copies of fungal | ST8927 | pCfB9213 |
| | CpEgt2x2 + | pathway with MET16 | | |
| | MET16 | integration | | |
| ST9583 | NcEgt1x2 + | Two copies of fungal | ST8927 | pCfB9214 |
| | CpEgt2x2 + | pathway with MTHFR | | |
| | MTHFR | chimera | | |
| ST9584 | NcEgt1-YI<− | Fungal pathway | ST6512 | pCfB9216 |
| | PrGPD::PrTEFin | | | |
| ST9687 | NcEgt1x2 + | Two copies of fungal | ST8927 | TRA |
| | CpEgt2x2 + | pathway, strain mutated | | resistance |
| | TRA^{R} | through β-(1,2,4,-triazol-3- | | |
| | | yl)-DL-alanine. | | |
| ST9689 | NcEgt1x2 + | Two copies of fungal | ST8927 | pCfB9374, |
| | CpEgt2x2 + | pathway with AGP2 knock- | | PR-26322 |
| | ΔAGP2 | out | | |
| ST9690 | NcEgt1x2 + | Two copies of fungal | ST8927 | pCfB9375, |
| | CpEgt2x2 + | pathway with TPO3 knock- | | PR-26324 |
| | ΔTPO3 | out | | |
| ST9691 | NcEgt1x2 + | Two copies of fungal | ST8927 | pCfB9376, |
| | CpEgt2x2 + | pathway with TPO4 knock- | | PR-26326 |
| | ΔTPO4 | out | | |
| ST9692 | NcEgt1x2 + | Two copies of fungal | ST8927 | pCfB9377, |
| | CpEgt2x2 + | pathway with AQR1 knock- | | PR-26328 |
| | ΔAQR1 | out | | |
| ST9693 | NcEgt1x2 + | Two copies of fungal | ST8927 | pCfB9384 |
| | CpEgt2x2 + | pathway with TPO1 | | |
| | TPO1 | integration | | |
| ST9694 | NcEgt1x2 + | Two copies of fungal | ST8927 | pCfB9385 |
| | CpEgt2x2 + | pathway with AtOCT1 | | |
| | AtOCT1 | integration | | |
| ST9695 | NcEgt1x2 + | Two copies of fungal | ST8927 | pCfB9386 |
| | CpEgt2x2 + | pathway with AtOCT7 | | |
| | AtOCT7 | integration | | |
| ST9696 | NcEgt1x2 + | Two copies of fungal | ST8927 | pCfB9387 |
| | CpEgt2x2 + | pathway with | | |
| | HsSLC22A12 | HsSLC22A12 integration | | |
| ST9697 | NcEgt1x2 + | Two copies of fungal | ST8927 | pCfB9388 |
| | CpEgt2x2 + | pathway with | | |
| | HsSLC22A16 | HsSLC22A16 integration | | |
| ST9698 | NcEgt1x2 + | Two copies of fungal | ST8927 | pCfB9389 |
| | CpEgt2x2 + | pathway with | | |
| | HsSLC22A32 | HSSLC22A32 integration | | |
| ST9699 | NcEgt1x2 + | Two copies of fungal | ST9929 | pCfB9390 |
| | CpEgt2x2 + | pathway, strain mutated | | |
| | TRA^{R} + | through β-(1,2,4,-triazol-3- | | |
| | MET14 + | yl)-DL-alanine, integration | | |
| | MET16 | of MET14 & MET16 | | |
| ST9700 | NcEgt1x2 + | Two copies of fungal | ST9929 | pCfB9391 |
| | CpEgt2x2 + | pathway, strain mutated | | |
| | TRA^{R} + | through β-(1,2,4,-triazol-3- | | |
| | MET14 + | yl)-DL-alanine, integration | | |
| | STP1 | of MET14 & STP1 | | |
| ST9701 | NcEgt1x2 + | Two copies of fungal | ST9909 | pCfB9391 |
| | CpEgt2x2 + | pathway, strain mutated | | |
| | TRA^{R} + | through β-(1,2,4,-triazol-3- | | |
| | MET16 + | yl)-DL-alanine, integration | | |
| | STP1 | of MET16 & STP1 | | |
| ST9702 | NcEgt1x2 + | Two copies of fungal | ST9699 | pCfB9391 |
| | CpEgt2x2 + | pathway, strain mutated | | |
| | TRA^{R} + | through β-(1,2,4,-triazol-3- | | |
| | MET14 + | yl)-DL-alanine, integration | | |
| | MET16 + | of MET14, MET16 & STP1 | | |
| | STP1 | | | |
| ST9703 | CpEgt2<− | Fungal pathway | ST6512 | pCfB9324 |
| | PrGPD::PrTEFin−> | | | |
| | NcEgt1-YI | | | |
| ST9909 | NcEgt1x2 + | Two copies of fungal | ST9687 | pCfB9390 |
| | CpEgt2x2 + | pathway, strain mutated | | |
| | TRA^{R} + | through β-(1,2,4,-triazol-3- | | |
| | MET16 | yl)-DL-alanine, integration | | |
| | | of MET16 | | |
| ST9910 | NcEgt1x2 + | Two copies of fungal | ST9687 | pCfB9391 |
| | CpEgt2x2 + | pathway, strain mutated | | |
| | TRA^{R} + | through β-(1,2,4,-triazol-3- | | |
| | STP1 | yl)-DL-alanine, integration | | |
| | | of STP1 | | |
| ST9911 | NcEgt1 + | One copy of fungal | ST8460 | pCfB9378, |
| | SpEgt2 + | pathway with ERG4 | | PR-26368 |
| | Δerg4 | deletion | | |
| ST9912 | NcEgt1 + | One copy of fungal | ST8460 | pCfB9198, |
| | SpEgt2 + | pathway with GCN4 | | PR-25131 |
| | Δgcn4_uORFs | upstream ORF deletion | | |
| ST9913 | NcEgt1 + | One copy of fungal | ST8460 | pCfB9379, |
| | SpEgt2 + | pathway with SPE2 | | PR-26388 |
| | Δspe2 | deletion | | |
| ST9914 | NcEgt1 + | One copy of fungal | ST8460 | pCfB9380, |
| | SpEgt2 + | pathway with STR2 | | PR-26390 |
| | Δstr2 | deletion | | |
| ST9915 | NcEgt1 + | One copy of fungal | ST8460 | pCfB9381, |
| | SpEgt2 + | pathway with URE2 | | PR-26392 |
| | Δure2 | deletion | | |
| ST9916 | NcEgt1 + | One copy of fungal | ST8460 | pCfB9207 |
| | SpEgt2 + | pathway with STP1 | | |
| | STP1 | integration | | |
| ST9917 | NcEgt1 + | One copy of fungal | ST8460 | pCfB9212 |
| | SpEgt2 + | pathway with MET14 | | |
| | MET14 | integration | | |
| ST9918 | NcEgt1 + | One copy of fungal | ST8460 | pCfB9213 |
| | SpEgt2 + | pathway with MET16 | | |
| | MET16 | integration | | |
| ST9919 | NcEgt1 + | One copy of fungal | ST8460 | TRA |
| | SpEgt2 + | pathway, strain mutated | | resistance |
| | TRA^{R} | through β-(1,2,4,-triazol-3- | | |
| | | yl)-DL-alanine. | | |
| ST9920 | CpEgt1 + | One copy of mixed | ST8474 | pCfB9378, |
| | MsEgt2 + | pathway with ERG4 | | PR-26368 |
| | Δerg4 | deletion | | |
| ST9922 | CpEgt1 + | One copy of mixed | ST8474 | pCfB9379, |
| | MsEgt2 + | pathway with SPE2 | | PR-26388 |
| | Δspe2 | deletion | | |
| ST9923 | CpEgt1 + | One copy of mixed | ST8474 | pCfB9380, |
| | MsEgt2 + | pathway with STR2 | | PR-26390 |
| | Δstr2 | deletion | | |
| ST9924 | CpEgt1 + | One copy of mixed | ST8474 | pCfB9381, |
| | MsEgt2 + | pathway with URE2 | | PR-26392 |
| | Δure2 | deletion | | |
| ST9926 | CpEgt1 + | One copy of fungal | ST8474 | pCfB9212 |
| | MsEgt2 + | pathway with MET14 | | |
| | MET14 | integration | | |
| ST9927 | CpEgt1 + | One copy of mixed | ST8474 | pCfB9213 |
| | MsEgt2 + | pathway with MET16 | | |
| | MET16 | integration | | |
| ST9928 | CpEgt1 + | One copy of mixed | ST8474 | TRA |
| | MsEgt2 + | pathway, strain mutated | | resistance |
| | TRA^{R} | through β-(1,2,4,-triazol-3- | | |
| | | yl)-DL-alanine. | | |
| ST9929 | NcEgt1x2 + | Two copies of fungal | ST9687 | pCfB9719 |
| | CpEgt2x2 + | pathway, strain mutated | | |
| | TRA^{R} + | through β-(1,2,4,-triazol-3- | | |
| | MET14 | yl)-DL-alanine, integration | | |
| | | of MET14 | | |
| ST10163 | NcEgt1x2 + | Two copies of fungal | ST9929 | pCfB9378, |
| | CpEgt2x2 + | pathway, strain mutated | | PR-26386 |
| | TRA^{R} + | through β-(1,2,4,-triazol-3- | | |
| | MET14 + | yl)-DL-alanine, integration | | |
| | Δerg4 | of MET14, deletion of | | |
| | | ERG4 | | |
| ST10165 | NcEgt1x2 + | Two copies of fungal | ST9929 | pCfB9379, |
| | CpEgt2x2 + | pathway, strain mutated | | PR-26388 |
| | TRA^{R} + | through β-(1,2,4,-triazol-3- | | |
| | MET14 + | yl)-DL-alanine, integration | | |
| | Δspe2 | of MET14, deletion of | | |
| | | SPE2 | | |
| ST10166 | NcEgt1x2 + | Two copies of fungal | ST9929 | pCfB9380, |
| | CpEgt2x2 + | pathway, strain mutated | | PR-26390 |
| | TRA^{R} + | through β-(1,2,4,-triazol-3- | | |
| | MET14 + | yl)-DL-alanine, integration | | |
| | Δstr2 | of MET14, deletion of | | |
| | | STR2 | | |
| ST10167 | NcEgt1x2 + | Two copies of fungal | ST9929 | pCfB9381, |
| | CpEgt2x2 + | pathway, strain mutated | | PR-26392 |
| | TRA^{R} + | through β-(1,2,4,-triazol-3- | | |
| | MET14 + | yl)-DL-alanine, integration | | |
| | Δure2 | of MET14, deletion of | | |
| | | URE2 | | |

Media and Yeast Cultivation Conditions

After transformation with plasmids, E. coli was grown on LB plates with 100 mg/l ampicillin. For the selection of yeast strains after modification with Cas9 plus gRNA, YPD plates supplemented with 200 mg/l G418 and/or nourseothricin (100 mg/l) were used. For Examples 1-3 yeast strains that were screened for ergothioneine production were grown in either Synthetic Complete (SC) medium with 20 g/l glucose and 1 g/l of histidine, cysteine and methionine for 48 hours, SC with 40 g/l glucose for 72 hours or SC with 60 g/l EnPump substrate, 0.6% reagent A for 72 hours at 30° C. and 250 rpm. The cells were inoculated at OD₆₀₀=0.5 in 24-deep-well plates. For Example 4, synthesis of ergothioneine over time by S. cerevisiae was also investigated by inoculating the strains at OD₆₀₀=0.5 and taking samples of the culture at set time intervals (every 8 and 24 hours of a day). The media used was SC medium with 40 g/l glucose, which was supplemented with various concentrations of histidine, cysteine and methionine to analyze the effect of precursor supplementation on the ergothioneine titer. For Examples 6-10, S. cerevisiae strains that were screened for ergothioneine production were grown in mineral medium containing 7.5 g/L (NH₄)₂SO₄, 14.4 g/L KH₂PO₄, 0.5 g/L MgSO₄.7H₂O, appropriate growth factors, 60 g/L EnPump 200 substrate and 0.6% reagent A for 72 hour at 30° C. and 250 rpm. For example 6 and 10, the cells were inoculated at OD₆₀₀=0.1 in 96-deep-well plates. For Example 7-9, the cells were inoculated at OD₆₀₀=0.1 in 24-deep-well plates. For example 11, S. cerevisiae and Yarrowia lipolytica that were screened for ergothioneine production were grown in either SC medium with 20 g/L glucose or SC medium with 60 g/L Enpump substrate and 0.6% reagent A for 72 hours at 30° C. and 250 rpm. The cells were inoculated at OD₆₀₀=0.1 in 96-deep-well plates.

### Creating a β-(1,2,4-triazol-3-yl)-DL-alanine resistant strain (HIS1 mutation strain)

To generate a histidine overproducing strain, 10 OD₆₀₀ units of ST8927 was plated onto a plate containing YNB—amino acids—(NH₄)₂SO₄+proline+0.25 mM β-(1,2,4-triazol-3-yl)-DL-alanine. After 5-7 days, 30 colonies were picked and screened in mineral medium containing 7.5 g/L (NH₄)₂SO₄, 14.4 g/L KH₂PO₄, 0.5 g/L MgSO₄.7H₂O, appropriate growth factors, 20 g/L glucose and 30 mM histidine. Colonies that did not grow were screened in mineral medium containing 7.5 g/L (NH₄)₂SO₄, 14.4 g/L KH₂PO₄, 0.5 g/L MgSO₄.7H₂O and 20 g/L glucose for their histidine and ergothioneine production. The cells were inoculated at OD₆₀₀=0.1 in 24-deep-well plates and incubated for 72 hour at 30° C. and 250 rpm. Colony 3 was chosen to be used as ST9687.

HPLC Analysis

Ergothioneine and histidine were quantified by HPLC. Intra- and extracellular concentrations of ergothioneine were determined separately, by measurement of ergothioneine in the supernatant and extraction of ergothioneine from cells based on a method from Alamgir et al., 2015. A 1 ml sample of fermentation broth was centrifuged at 3000×g for 5 min and the supernatant was removed and stored at −4° C. until the analysis of extracellular ergothioneine. The remaining cell pellet was washed twice with MilliQ water and then resuspended in 1 ml water. The cells were boiled at 94° C. for 10 minutes and then vortexed at 1600 rpm for 30 minutes using a DVX-2500 Multi-Tube Vortexer from VWR. After centrifugation at 10,000×g for 5 minutes, the supernatant was taken and analyzed for intracellular ERG concentration using HPLC. Total ergothioneine concentration was determined by not separating the cells from the broth before boiling the sample. The full samples (fermentation broth and cells) were treated as described above for the boiling, vortexing and centrifuging. After centrifugation, the supernatant was taken to analyze the total ergothioneine concentration by HPLC. For HPLC analysis, the Dionex Ultimate 3000 HPLC system with the analysis software Chromeleon was used. Samples were run on a Cortects UPLC T3 reversed-phase column (particle size 1.6 μm, pore size 120 Å, 2.1×150 mm). The flow rate was 0.3 ml/min, starting with 2.5 minutes of 0.1% formic acid, going up to 70% acetonitrile, 30% 0.1% formic acid at 3 minutes for 0.5 minutes, after which 100% 0.1% formic acid was run from minute 4 to 9. Ergothioneine was detected at a wavelength of 254 nm.

Propidium Iodide Staining and Flow Cytometry Analysis

1 ml sample of cell culture was taken from the yeast cultivation. These were washed two times with phosphate-buffered saline (PBS), subsequently resuspended in 0.5 μg/ml propidium iodide in PBS and incubated for 20 minutes at room temperature. After incubation, the cells were washed two times with PBS and then the percentage of PI stained cells was determined using a MACSQuant VYB system. Analysis was performed using the FlowJo software.

Simulated Fed-Batch Production of Ergothioneine

Solutions and media: Trace metal solution contained: 4.5 g/L CaCl₂.2H2O, 4.5 g/L ZnSO₄.7H₂O, 3 g/L FeSO₄.7H₂O, 1 g/L H₃BO₃, 1 g/L MnCl₂.4H₂O, 0.4 g/L Na₂MoO₄.2H₂O, 0.3 g/L CoCl₂.6H₂O, 0.1 g/L CuSO₄.5H₂O, 0.1 g/L KI and 15 g/L EDTA. Vitamin solution contained: 50 mg/L biotion, 200 mg/L p-aminobenzoic acid, 1 g/L nicotinic acid, 1 g/L Ca-pantotenate, 1 g/L pyridoxine-HCl, 1 g/L thiamine-HCl and 25 g/L myo-inositol. The simulated fed-batch medium consisted of 7.5 g/L (NH₄)₂SO₄, 14.4 g/L KH₂PO₄, 0.5 g/L MgSO₄, 1 g/L yeast extract, 2 mL/L trace metals solution, 1 mL/L vitamins solution and 200 g/L Enpump substrate. All components were weighed, dissolved in water and subsequently sterile filtered before use.

Simulated fed-batch production of ergothioneine: A single colony from a YPD plate with ST10165 (NcEgt1×2+CpEgt2×2+TRA^{R}+MET14+Δspe2) was used to inoculated 5 mL of mineral medium containing 7.5 g/L (NH4)2SO4, 14.4 g/L KH2PO4, 0.5 g/L MgSO4.7H₂O, appropriate growth factors and 20 g/L glucose in a 13 mL preculture tube. The tube was incubated at 30° C. and 250 rpm overnight. This overnight culture was transferred into two times 50 ml mineral medium in a 500 mL baffled shake flask. The shake flask was then incubated overnight at 30° C. and 250 rpm. The cultures were then centrifuged at 3,000×g for 5 minutes. The cells were resuspended in 25 mL sterile MilliQ water and subsequently combined. Enough cells for a cell dry weight of 5, 10, 20 and 40 g/L in 7 mL of solution were each transferred to a 15 mL Falcon tube and centrifuged at 3,000×g for 5 minutes. The cells were then resuspended in 7 mL simulated fed-batch medium. In a 24 deep-well plate, 20 different conditions were set-up. The staring cell dry weight was either 5, 10, 20 or 40 g/L and the concentration of reagent A was either 0.4%, 0.6%, 0.8%, 1.0% or 1.2%. For each of these conditions, 1 mL of the simulated fed-batch medium with the correct starting cell dry weight was added to a well, after which the appropriate concentration of reagent A was added. The cells were then incubated at 30° C. and 250 rpm for 188 hours. After 68 and 140 hours, the same amount of reagent A as the starting concentration was added to the well to avoid loss of enzymatic activity. After 188 hours, the total ergothioneine production for each condition was analyzed by HPLC:

### Example 2—Results: Integration of the Ergothioneine Biosynthetic Pathway in Yeast

Using the sequence of Egt1 for N. crassa (Genbank accession: XP_956324.3) in a

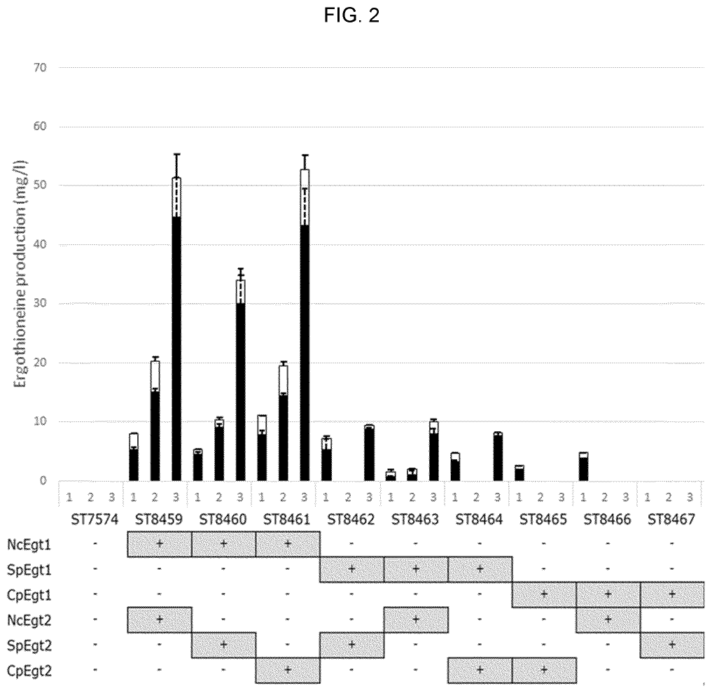

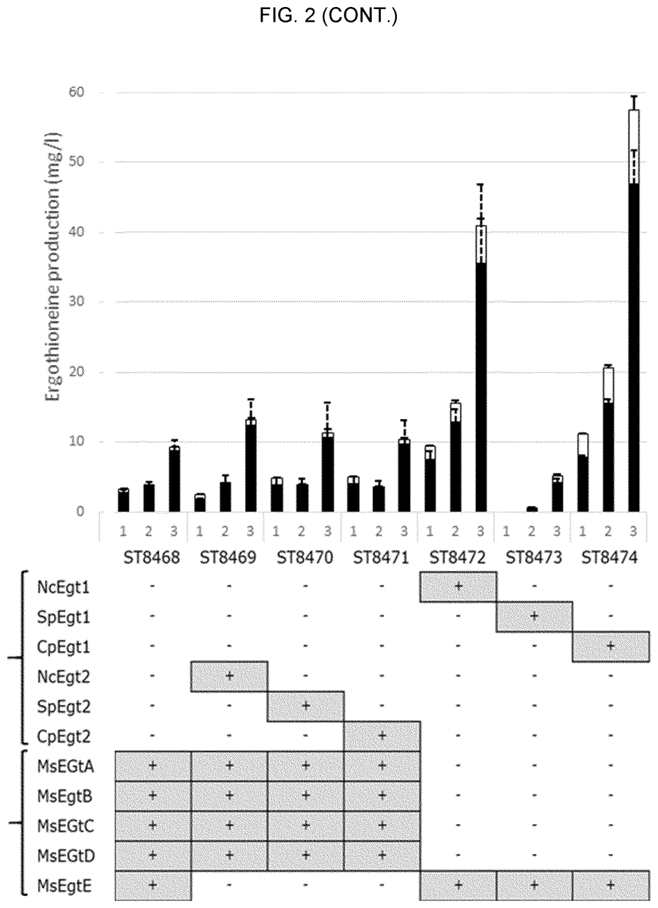

BLAST search, we have identified the Egt1 homologues in C. purpurea and S. pombe (Genbank accession: CCE33591.1 and NP_596639.2). Similarly, Egt2 from S. pombe (Genbank accession: NP_595091.1) was used to find the Egt2 homologues in N. crassa and C. purpurea (Genbank accession: XP_001728131.1 and CCE33140.1). The amino acid sequences for M. smegmatis genes EgtA, EgtB, EgtC, EgtD and EgtE were taken from Genbank as well (Genbank accession: AFP42520.1, WP_011731158.1, WP_011731157.1, WP_011731156.1, ABK70212.1). All the genes were generated as synthetic DNA strings, codon-optimized for S. cerevisiae, except for Egt1 and Egt2 from S. pombe, as those were amplified from a genomic DNA extract. In total, 16 pathway variants were assembled, of which 9 were fungal, 1 bacterial, and 6 mixed fungal-bacterial (Table 2). The 16 resulting yeast strains were cultivated in deep-well plates under different conditions and the intra- and extracellular concentrations of ergothioneine were measured (FIG. 2).

Overall, the production of ergothioneine for the different combinations was between 0 and 57 mg/L of yeast culture. Strain ST8461, expressing Egt1 from Neurospora crassa and Egt2 from Claviceps purpurea, both enzymes from the eukaryotic ERG biosynthesis pathway, was one of the best performing strains in all three conditions and was selected for further studies.

### Example 3—Results: Ergothioneine Transporter

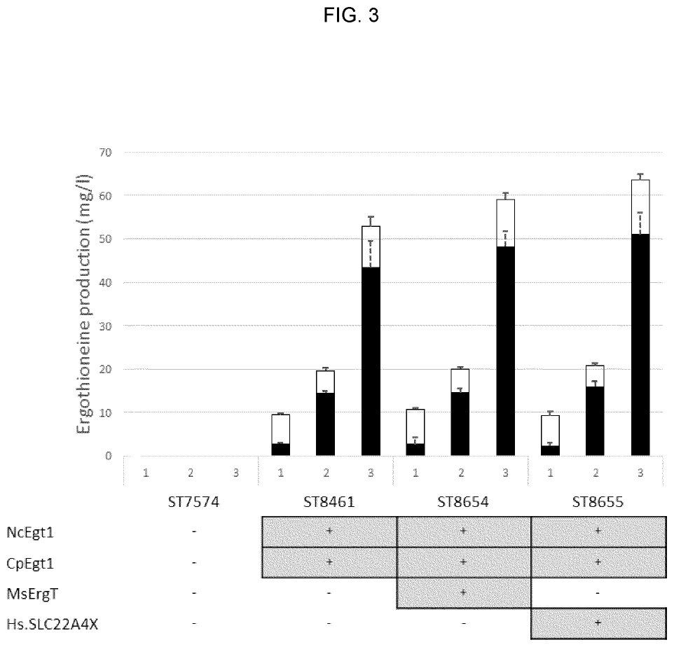

As about half of the produced ERG was retained in the cell, we investigated whether export of ERG from the yeast cells may be limiting the production, at least in part. Estimating the wet weight concentration at 0.37 mg/g wet weight yeast cells (taken from measurements in SC+20 g/l glucose+1 g/l His/Cys/Met), the concentration of ERG inside the cells would be 1.75 mM, or 120-fold higher than that in the broth. As M. smegmatis is known to secrete ergothioneine to levels up to 4 times the intracellular concentration, given in pg/10⁵ CFU, we speculated there must be a transporter for ERG in its genome. Therefore, the biosynthetic ERG cluster in this organism was investigated. Besides the 5 known biosynthetic Egt genes, the cluster contained 1 transmembrane protein, which we hypothesized could be an ERG transporter. To test the effect of the product of this gene on ERG production in yeast, the high-producing strain ST8461 was engineered to express either this putative transporter or the known ergothioneine transporter SLC22A4 (SCL22A4X) from humans (Grundemann et al., 2005). Both transporters showed slightly increased titers when using simulated fed batch medium (FIG. 3), but no change was observed in the intra- to extracellular ergothioneine ratio. An important note is that the human ergothioneine transporter SLC22A4X acts as an importer in human cells, but shows a slight effect on the production titer in simulated fed batch medium here.

### Example 4—Supplementation with Amino Acids

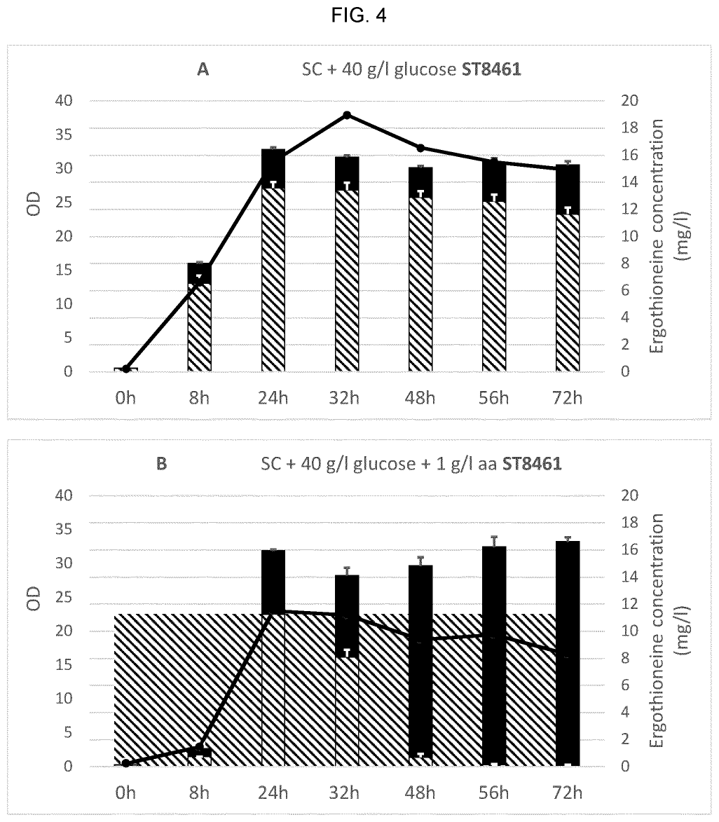

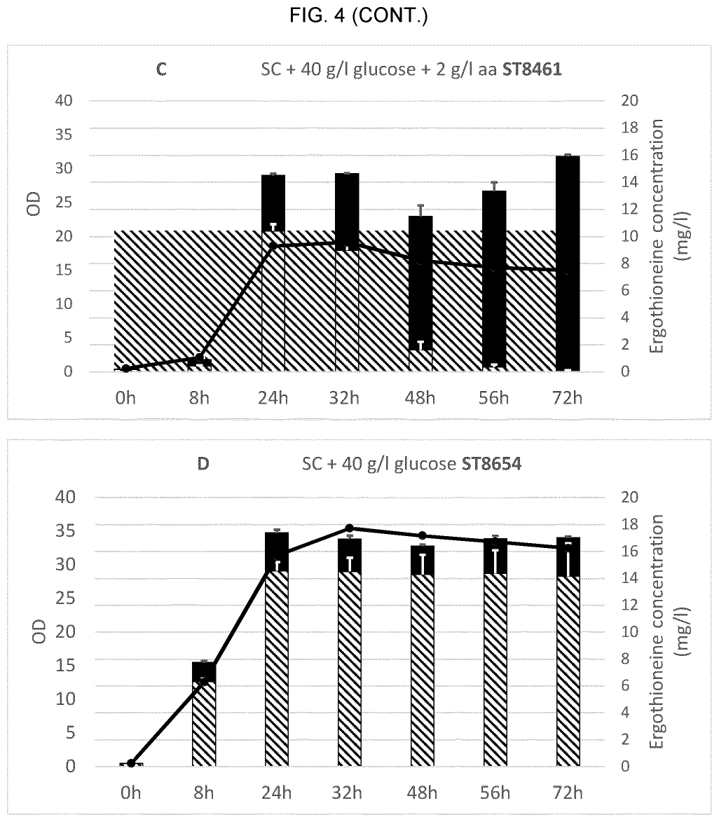

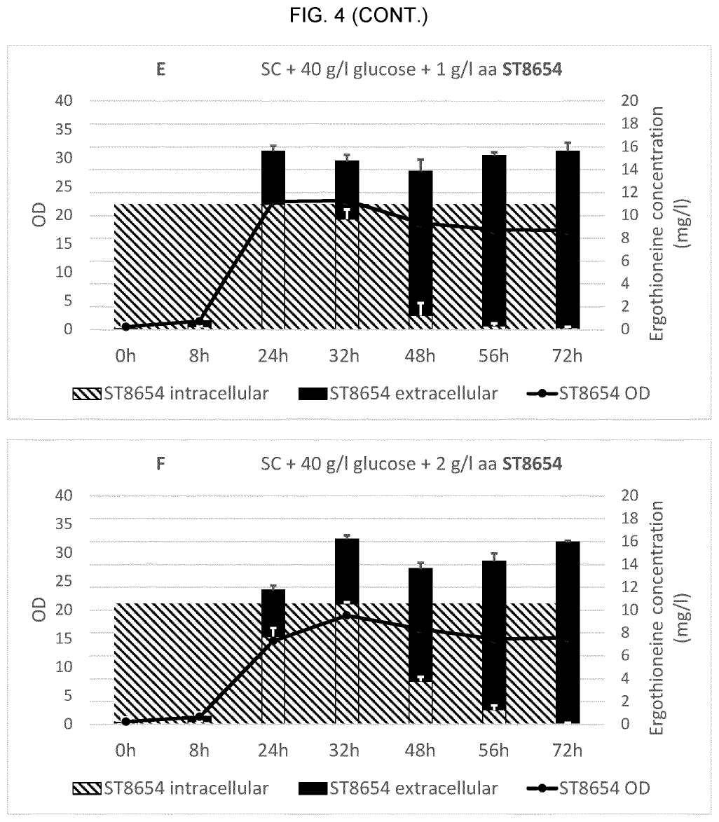

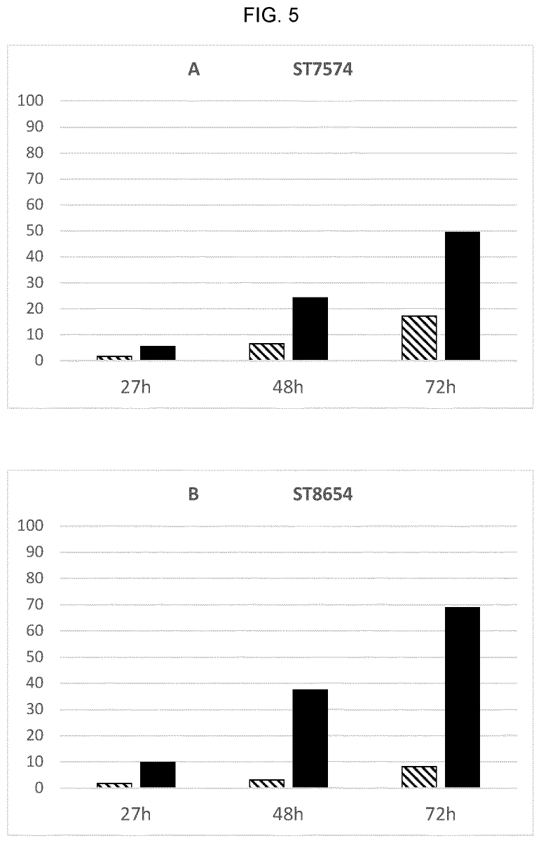

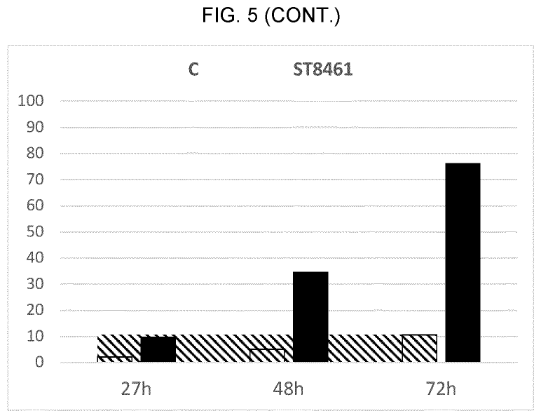

In order to further improve the titer of ergothioneine, the effect of medium supplementation with the three amino acids that serve as precursors for ergothioneine was further investigated. We tested 3 strains, a non-producing strain (ST7574), a producing strain (ST8461) and a producing strain with the ergothioneine transporter from M. smegmatis (ST8654). The experiments were performed in shake flasks with synthetic complete medium, supplemented with 1 g/L or 2 g/L of each L-methionine, L-cysteine and L-histidine. Biomass growth and production of ERG were monitored over 72 hours (FIG. 3). Ergothioneine accumulated primarily in the first 24 hours of cultivation, which would correspond to the exponential growth on glucose, reaching ca. 16 mg/L in both producing strains, independent of any amino acid supplementation. The supplementation, however, affected the cellular growth, with the final OD being approximately 46 and 52% lower when correspondingly 1 g/L or 2 g/L of amino acids were added. No degradation of ergothioneine was observed; however, surprisingly, there was a large variation in intracellular vs extracellular distribution of ERG depending on the addition of amino acids. Specifically, the addition of amino acids promoted the excretion of ergothioneine in the stationary phase. We hypothesized this was due to cell death. Indeed propidium iodide staining of cells sampled at 24 hours, showed an increase in the fraction of dead cells from 9 to 70%, when amino acids were added at concentrations of 1 g/L (FIGS. 4 and 5).

### Example 5—Production of Ergothioneine in Diploid Brewer's Yeast

Solutions and Media

Trace metal solution contained: 4.5 g/l CaCl₂.2H₂O, 4.5 g/l ZnSO₄.7H₂O, 3 g/l FeSO₄.7H₂O, 1 g/l H₃BO₃, 1 g/l MnCl₂.4H₂O, 0.4 g/l Na₂MoO₄.2H₂O, 0.3 g/l CoCl₂.6H₂O, 0.1 g/l CuSO₄.5H₂O, 0.1 g/l KI and 15 g/l EDTA. Vitamin solution contained: 50 mg/l biotion, 200 mg/l p-aminobenzoic acid, 1 g/l nicotinic acid, 1 g Ca-pantotenate, 1 g/l pyridoxine-HCl, 1 g/l thiamine-HCl and 25 g/l myo-inositol. The mineral media consisted of 4.4 g/l (NH₄)₂SO₄, 14.4 g/l KH₂PO₄, 0.5 g/l MgSO₄, 20 g/l glucose, 400 mg/l arginine, 400 mg/l histidine, 400 mg/l methionine, 4 mg/l pyridoxine, 2 ml/l trace metals solution and 1 ml/l vitamins solution. All components were weighed, dissolved in water and subsequently sterile filtered before use. The feeding medium consisted of 415 g/l glucose, 7.5 g/l (NH₄)₂SO₄, 14.4 g/l KH2PO4, 0.5 g/l MgSO4, 7.5 g/l arginine, 7.5 g/l histidine, 7.5 g/l methionine, 0.5 g/l pyridoxine, 4 ml/l trace metals solution, 2 ml/l vitamin solution and 1 ml/l antifoam. All components were weighed, dissolved using slightly heated water and subsequently sterile filtered prior to use.

Controlled Fermentation

A single colony from a YPD plate with ST8927 colonies was used to inoculate 5 ml of minimal media in 13-ml tube. The tube was incubated at 30° C. and 250 rpm overnight. This overnight culture was transferred into 95 ml mineral medium in 500 ml buffled shake flask. The shake flask was then incubated overnight at 30° C. and 250 rpm. 40 ml of this dense culture was used to inoculate 60 ml mineral medium in a new 500 ml buffled shake flask. Two shake flasks were prepared this way. These shake flasks were incubated at 30° C. and 250 rpm for 4 hours, the content of both shake flasks was combined, centrifuged at 3,000×g for 5 min. The supernatant was discarded, the pellet was washed with 25 ml sterile water, resuspended and centrifuged as before. The supernatant was discarded and the pellet resuspended in 10 ml mineral medium. This was then used to inoculate 0.5 l mineral medium in a 1 l Sartorius bioreactor. The starting OD₆₀₀ was 0.85. The stirring rate was set at 500 rpm, the temperature was kept at 30° C., and pH was maintained at pH 5.0 using 2 M KOH and 2 M H₂SO₄. The feeding was started as soon as CO₂ in the off-gas decreased by 50%. The initial feed rate was set at 0.6 g glucose h-1, linearly increasing to 2.5 g glucose h-1 over the span of 25.5 hours. After that, the feed was set at a constant 1.4 g glucose h-1 and 17.8 hours later, the feeding rate was set to a constant 2.9 g glucose h⁻¹. The feed was stopped at 84 hours. At 60.5 and 75.5 hours, 2 g (NH₄)₂SO₄ was added as a sterile 100 g/l solution. At 60.5 and 73.5 hours, 0.5 g MgSO₄ was added as a sterile 50 g/l solution, 4 ml sterile trace metals solution was added and 2 ml sterile vitamin solution was added.

Results

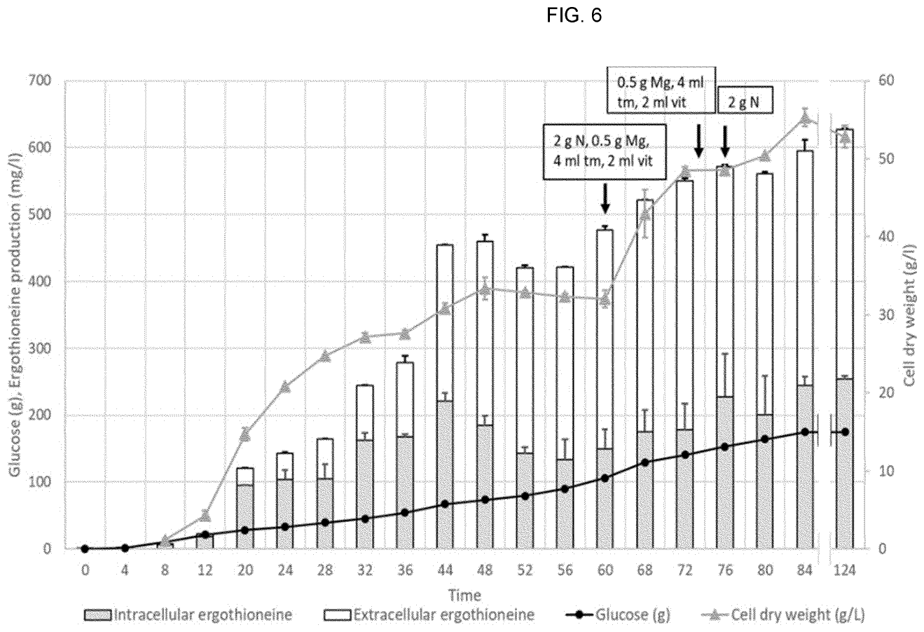

Ergothioneine was quantified by HPLC as in Example 1. Cell dry weight and glucose concentrations were measured as in Borodina et al., 2015. The mean data from duplicate bioreactors is shown on FIG. 6. The final total concentration of ergothioneine was 0.63 g/l.

### Example 6—Further Metabolic Engineering by Single Target Modifications—Target Screening in ST8927

Examples 1 to 5 are directed to metabolic engineering of the ergothioneine biosynthesis pathway. Next further metabolic engineering were conducted to increase the production of ergothioneine further. From here on, the experiments in the examples are performed using mineral medium (as described in the materials and methods) rather than SC medium, with the exception of example 11.

The inventors rationally selected targets that might improve ergothioneine production further. Targets within the nitrogen catabolite repression and the transport of nitrogen backgrounds were chosen to increase the availability of nitrogen for the precursors S-adenosylmethionine (SAM), histidine and cysteine. Furthermore, the general amino acid control was targeted to improve the synthesis of all the precursors. Individual amino acid biosynthesis pathways were also chosen to be activated. Lastly, as both SAM and cysteine incorporate sulfur, targets within the sulfur assimilation pathway were also chosen.

Thus, the following pathways were additionally modified:
Nitrogen catabolite repression
Transport of nitrogenous compounds
General amino acid control
Individual amino acid biosynthesis pathways
Sulfur assimilation pathway

The genetic edits for each target in Table 2 were inserted in strain ST8927 (two copies of NcEgt1 and two copies of CpEgt2) and screened in 96-deep well plates using mineral medium.

TABLE 2

| | | |
|---|---|---|
| Target | Type of edit | Reasoning |

Nitrogen catabolite repression (NCR)

| | | |
|---|---|---|
| URE2 | Deletion | Derepression of NCR controlled genes |
| ARG82 | One copy | Upregulation improves derepression of NCR |
| | integration | controlled genes |

Transport of nitrogenous compounds

| | | |
|---|---|---|
| VBA1 | Deletion | Decreases transport of histidine to vacuole |
| VBA2 | Deletion | Decreases transport of histidine to vacuole |
| VBA3 | Deletion | Decreases transport of histidine to vacuole |
| PET8 | Deletion | Deletion of SAM transport into vacuole |
| SSY1 | One copy | Part of SPS sensing mechanism, could increase |
| | integration | nitrogen transport into cell |
| GRR1 | One copy | Part of SPS sensing mechanism, could increase |
| | integration | nitrogen transport into cell |
| YCK2 | One copy | Part of SPS sensing mechanism, could increase |
| | integration | nitrogen transport into cell |
| STP1 | One copy | Part of SPS sensing mechanism, could increase |
| | integration | nitrogen transport into cell |

General amino acid control

| | | |
|---|---|---|
| GCN2 | Mutation (E803V) | Increases GCN4 activation, derepression of amino |
| | | acid biosynthesis genes |
| GCN4 | Deletion of leader | Constitutive activation, derepression of amino acid |
| | or upstream start | biosynthesis genes |
| | codons | |
| PET18 | Deletion | Derepression of amino acid biosynthesis genes |

Arginine biosynthesis

| | | |
|---|---|---|
| ARG81 | Deletion | Upregulated arginine biosynthesis |

Histidine biosynthesis

| | | |
|---|---|---|
| BAS1- | Linked chimera | Activates histidine biosynthesis |
| PHO2 | | |
| TRA^{R} | β-(1,2,4-triazol-3- | Overproduction of histidine |
| | yl)-DL-alanine | |
| | resistance | |

Cysteine biosynthesis

| | | |
|---|---|---|
| CYS3 | One copy | Increase synthesis of cysteine from homocysteine |
| | integration | |
| CYS4 | One copy | Increase synthesis of cysteine from homocysteine |
| | integration | |
| STR2 | Deletion | Decrease conversion of cysteine towards |
| | | homocysteine |
| STR3 | Deletion | Decrease conversion of cysteine towards |
| | | homocysteine |
| GSH1 | Deletion | Decrease conversion of cysteine towards |
| | | glutathione |

S-adenosylmethionine (SAM) biosynthesis

| | | |
|---|---|---|
| SAM2 | One copy | Increases SAM production |
| | integration | |
| GLC3 | Deletion | Increases SAM pool |
| SPE2 | Deletion | Increases SAM pool |
| ERG4 | Deletion | Increases SAM pool |
| MTHFR | Chimera | Removes feedback resistance of MET13 |

Sulfur assimilation pathway

| | | |
|---|---|---|
| MET4 | One copy | Increases expression of sulfur assimilation |
| | integration | pathway enzymes |
| MET14 | One copy | Increases part of sulfur assimilation pathway |
| | integration | |
| MET16 | One copy | Increases part of sulfur assimilation pathway |
| | integration | |

Results

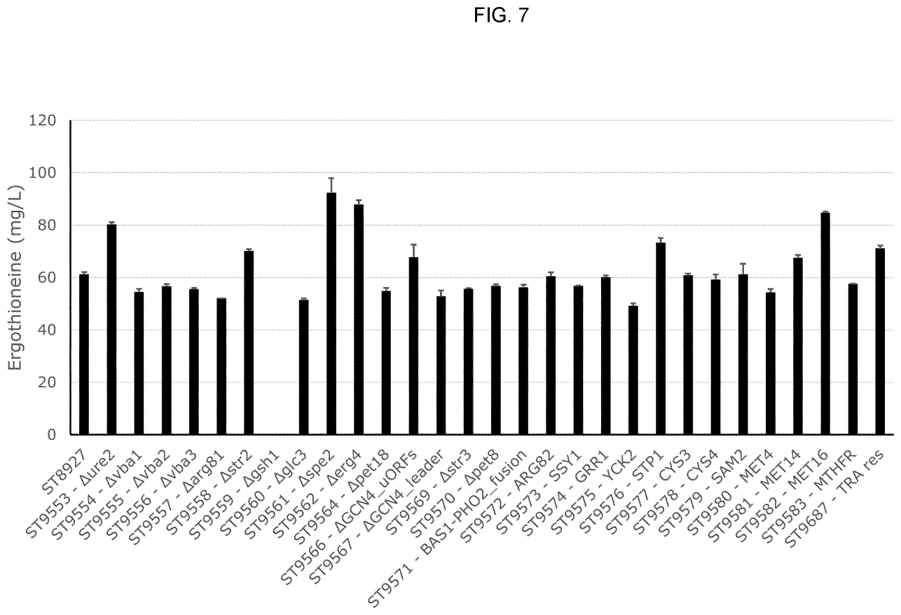

Nine out of 29 targets improved the ergothioneine production, see FIG. 7. These targets are the deletion of URE2, STR2, SPE2, ERG4 and the upstream start codons of GCN4; the integration of an extra copy of STP1, MET14 and MET16; and using β-(1,2,4-traizol-3-yl)-DL-alanine resistance to overproduce histidine. The deletion of ERG4 and SPE2 were particularly effective. These deletions increase the S-adenosylmethionine (SAM) pool and would also be useful in the production of other compounds requiring SAM in cell factories.

### Example 7—Combining Genetic Modifications—Histidine Overproduction Combined with Expression or Overexpression of STP1, MET14 and/or MET16

Example 6 showed that some of the genetic edits that improve ergothioneine production are from similar pathways and or the targets adjust pathways that interlink (e.g. homocysteine is a precursor for SAM and cysteine). Thus, it was next investigated whether the genetic edits found in Example 6 could further increase ergothioneine production when combined.

The ergothioneine production strain ST9687 (having two copies of NcEgt1 and two copies of CpEgt2 and which overproduces histidine due to β-(1,2,4-traizol-3-yl)-DL-alanine resistance) was used to integrate different combinations of STP1, MET14 and MET16 genes.

Results

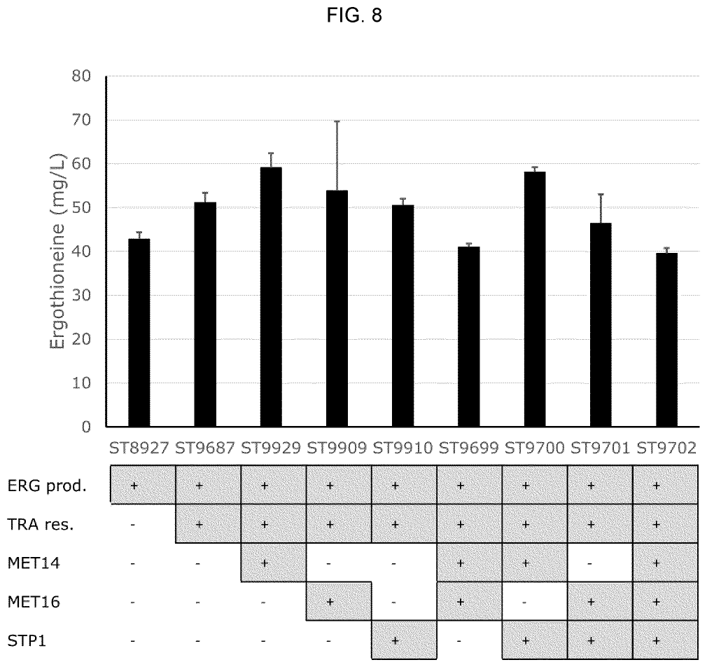

FIG. 8 shows the results. ST9687, which overproduces histidine, showed significant higher production of ergothioneine compared to ST8927. ST8927 was capable of producing at least 43 mg/L ergothioneine. ST9687 was capable of producing at least 59 mg/L ergothioneine. By combining histidine overproduction with MET14 integration increased the ergothioneine production (ST9929) the most. However, additional combinations (on top of the histidine overproduction and MET14 integration) did not increase the production further.

### Example 8—Combining Genetic Modifications—Histidine Overproduction and MET14 Combined with Deletions of ERG4, SPE2, STR2 and URE2

Example 7 showed increased ergothioneine production in strain ST9929 having histidine overproduction and MET14 integration. Subsequently, the deletions of ERG4, SPE2, STR2 and URE2 were added on top of strain ST9929.

Results

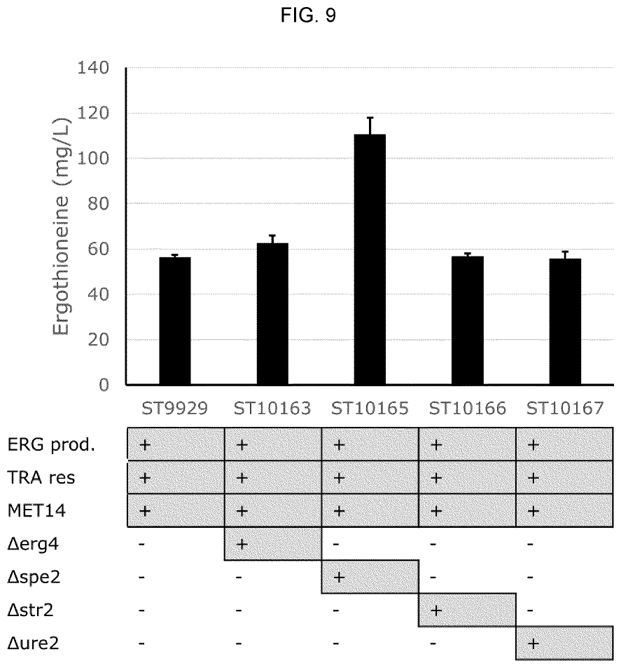

The results of this are shown in FIG. 9. Both ERG4 and SPE2 increased the ergothioneine further when combined with the histidine overproduction and MET14 integration. Both Examples 7 and 9 clearly show that combining the genetic edits found in Example 6 can further increase the ergothioneine production of the strain ST8927 by increasing the supply of several precursors simultaneously.

### Example 9—Further Testing of Transporters for Ergothioneine Production

Ten more transporter edits were tested to improve ergothioneine production. These transporters were integrated in the ST8927 strain (two copies of NcEgt1 and CpEgt2).

The transporters Agp2, Tpo3, Tpo4 and Aqr1 from S. cerevisiae were deleted; the transporter Tpo1 of S. cerevisiae, OCT1 and OCT7 of Arabidopsis thaliana, SLC22Al2, SLC22A16 and SLC22A32 of Homo sapiens were integrated individually in each strain.

Results

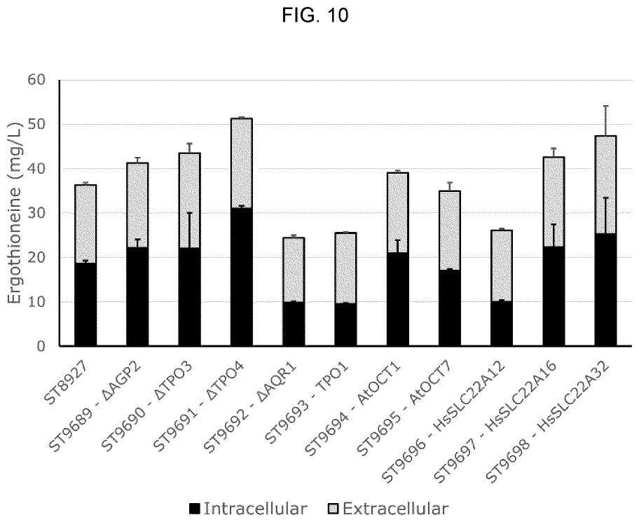

The deletion of TPO4 of S. cerevisiae increased the ergothioneine production. ST9691 was capable of producing at least 51 mg/L ergothioneine. See FIG. 10. This most likely leads to an accumulation of spermidine and spermine, reducing the need for SAM in the production of pantothenate. On the contrary, deletion of AQR1 and integration of TPO1 decreased the ergothioneine production (See FIG. 10). From this, it can be concluded that the deletion of TPO1 increases ergothioneine production for the same reason as the deletion of TPO4 increases ergothioneine production. AQR1 is a transporter that is involved in the excretion of excess amino acids. The decrease in ergothioneine production caused by the deletion of AQR1 can thus be explained by a reduced transport of ergothioneine out of the cell. Therefore, integration of AQR1 may increase ergothioneine productivity of the strain.

### Example 10—Target Confirmation in Other Ergothioneine Producing Enzyme Combinations

To confirm the effect the genetic edits have on ergothioneine production, the genetic edits found in Example 6 were also introduced in other strains with different ergothioneine production enzymes. All of the genetic edits were introduced in the strain ST8460 (one copy of NcEgt1 and SpEgt2), while a subset of the edits (Δerg4, Δspe2, Δstr2, Δure2, MET14 and MET16) were introduced in strain ST8474 (one copy of CpEgt1 and MsEgtE).

Results

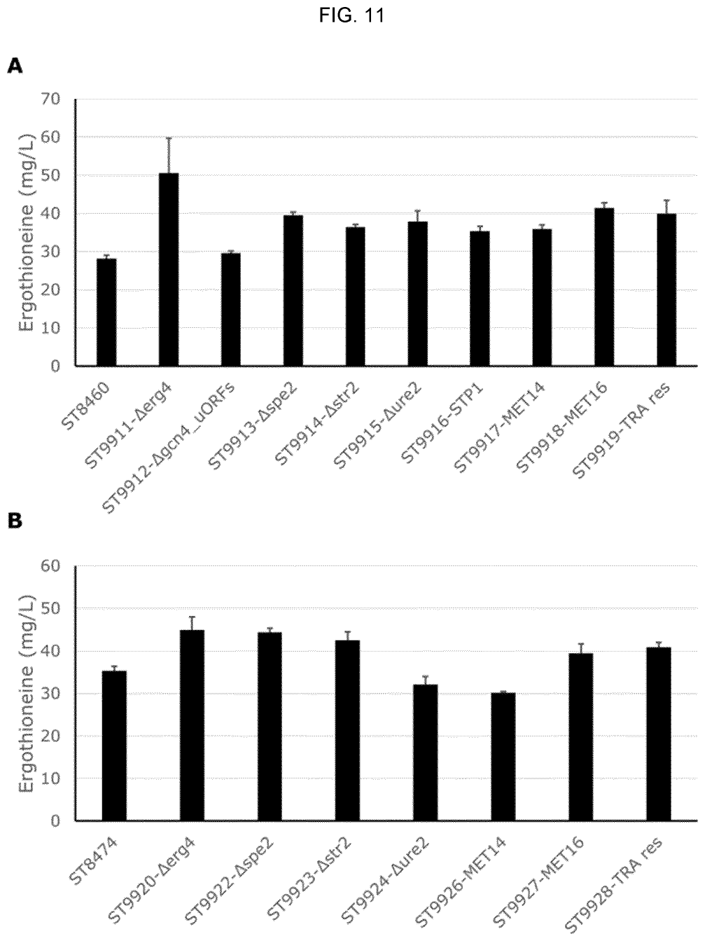

While all of the genetic edits showed an increase in ergothioneine production in strain ST8460 (FIG. 11A), the deletion of URE2 and the integration of MET14 did not increase ergothioneine production in ST8474 as seen in FIG. 11B. This could potentially be caused by a different activity of CpEgt1+MsEgtE, leading to different requirements of the precursor supply.

### Example 11—Ergothioneine Production in Other Yeasts

We wanted to show that the best performing enzyme combination for ergothioneine production found in Example 2 can also efficiently produce ergothioneine in other yeasts. To that end, we expressed NcEgt1 and CpEgt2 under the strong constitutive promoters TEFintron and GDP (both variations were made and tested) in Yarrowia lipolytica. To compare S. cerevisiae and Y. lipolytica, ST8461 (one copy of NcEgt1 and CpEgt2) and the two Y. lipolytica strains were cultured in SC medium with 20 g/L glucose (batch conditions) and SC medium with 60 g/L Enpump substrate+0.6% reagent A (simulated fed-batch conditions).

Results

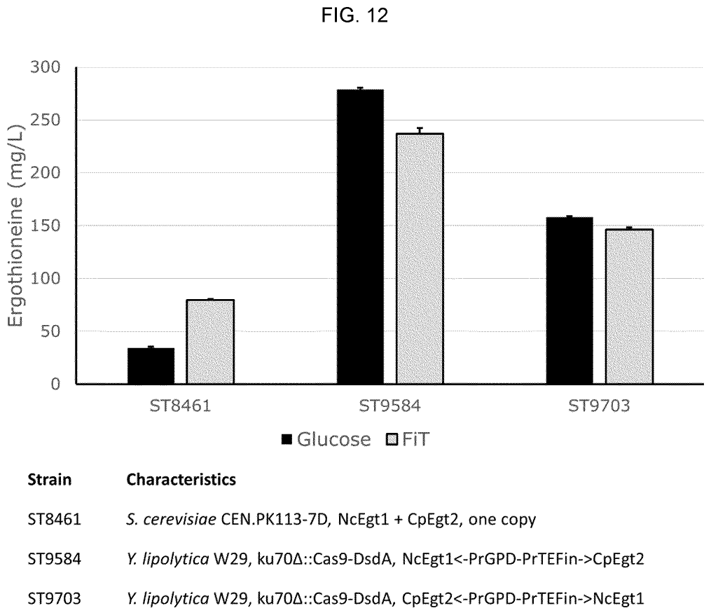

FIG. 12 shows that Y. lipolytica can produce up to 278 mg/L ergothioneine under batch conditions and up to 236 mg/L in simulated fed-batch conditions, compared to the 34 mg/L and 78 mg/L for these conditions respectively by S. cerevisiae. This shows ergothioneine can feasible be produced in a variety of yeasts, and that Y. lipolytica in particular is a promising host for ergothioneine production.

### Example 12—Simulated Fed-Batch Production of Ergothioneine

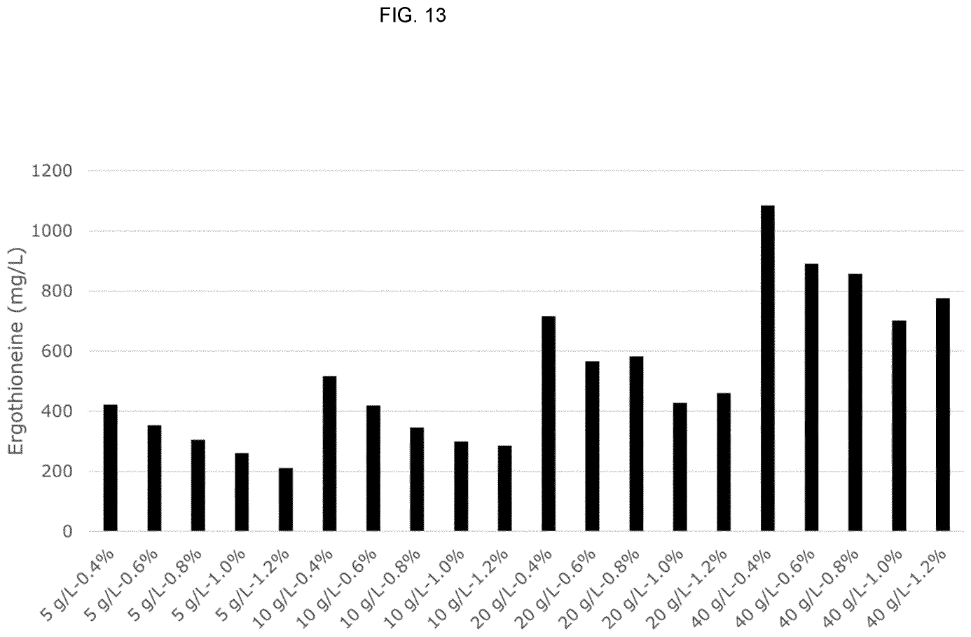

To investigate the ergothioneine production capabilities of our strain ST10165 (NcEgt1×2+CpEgt2×2+TRA^{R}+MET14+Δspe2), we inoculated the strain in simulated fed-batch medium (mineral medium with 1 g/L yeast extract and 200 g/L Enpump substrate) at different starting cell dry weight concentrations. By varying the concentration of the enzyme (reagent A) in each of these starting cell dry weight conditions, the combinations of starting cell dry weight and reagent A concentration can be screened for the best ergothioneine production. As shown in FIG. 13, 40 g/L of starting cell dry weight with 0.4% reagent A resulted in an ergothioneine production of 1.1 g/L.

### Example 13—Histidine Overproduction Strain

To increase ergothioneine production, β-(1,2,4-triazol-3-yl)-DL-alanine (TRA) was used to generate a strain with increased histidine production. TRA is an amino acid analogue that is toxic to the cells. When 0.25 mM TRA is added to a plate made with yeast nitrogen base with amino acids and ammonium sulfate and proline as the main nitrogen source, the cells have to (i) start overproducing histidine by removing feedback inhibition on the pathway, or (ii) the cells need to remove the uptake of TRA through the histidine transporter in order to grow. When either of these two options happens, the cells are resistant to β-(1,2,4-triazol-3-yl)-DL-alanine (TRA^{R}). The resulting strains have to then be screened using medium containing a toxic amount of histidine (30 mM) to differentiate between strains containing mutations in the histidine transporter or strains overproducing histidine. The strain that grow have their histidine transporter mutated and can be discarded. The overproduction in the strain that don't grow in medium containing 30 mM histidine is attributed to changes in the HIS1 locus, as shown through the mating of TRA^{R} haploids with his1⁻ temperature sensitive haploids in Rasse-Messenguy et al. 1973

To this end, ST8927 was plated on a plate containing TRA to generate various TRA resistant mutants. After screening in 30 mM histidine, colonies number 1, 2, 3, 4, 5, 10, 14, 25 and 28 were determined to not have mutations in the transport of histidine and could be screened for their histidine and ergothioneine production in mineral medium.

Results

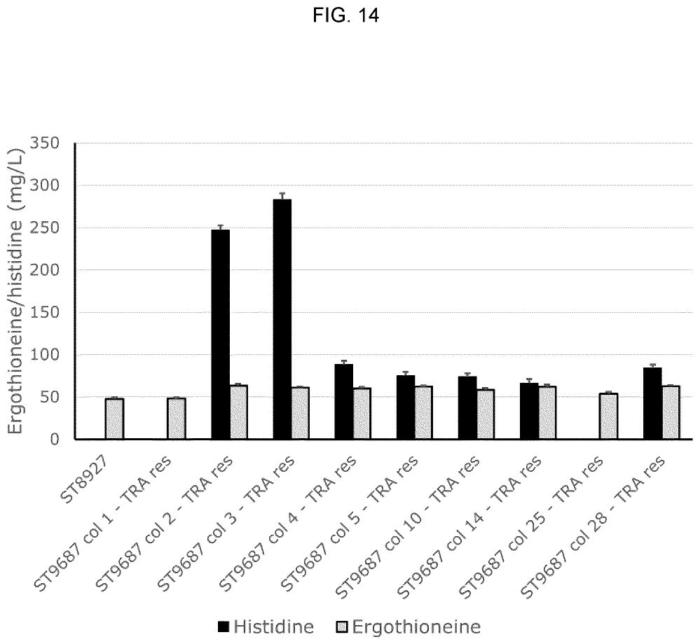

FIG. 14 shows the ergothioneine and histidine production of the selected colonies. ST9687 col 3 was capable of producing 283 mg/L histidine. Colony 3 was chosen to be used in further engineering efforts.

### REFERENCES

Alamgir, K. M., Masuda, S., Fujitani, Y., Fukuda, F. & Tani, A. Production of ergothioneine by Methylobacterium species. Front. Microbiol. 6, (2015).

Borodina, I. Kildegaard, K. R., Jensen, N. B., Blicher, T. H., Maury, J., Sherstyk, S., Schneider, K., Lamosa, P., Herrård, M. J., Rosenstand, I., Öberg, F., Forster, J., Nielsen, J. Metab. Eng. 27, 57-64 (2015).

Grundemann, D. et al. Discovery of the ergothioneine transporter. Proc. Natl. Acad. Sci. 102, 5256-5261 (2005).

Holkenbrink, C., Dam, M. I., Kildegaard, K. R., Beder, J., Dahlin, J., Belda, D. D., Borodina, I. (2018). EasyCloneYALI: CRISPR/Cas9-Based Synthetic Toolbox for Engineering of the Yeast Yarrowia lipolytica. Biotech. J., 13 (9), 1-8, doi: 10.1002/biot.201700543.

Jessop-Fabre, M. M. et al. EasyClone-MarkerFree: A vector toolkit for marker-less integration of genes into Saccharomyces cerevisiae via CRISPR-Cas9. Biotechnol. J. 11, 1110-1117 (2016).

Pinson, B., Kongsrud, T. L., Ording, E., Johansen, L., Daignan-Fornier, B., Gabrielsen, O. S. (2000). Signaling through regulated transcription factor interaction: mapping of a regulatory interaction domain in the Myb1-related Bas1p. Nucl. Acids Res., 28 (23), 4665-4673, doi: 10.1093/nar/28.23.4665

Rasse-Messenguy, F., Fink, G. R., (1973). Feedback-Resistant Mutants of Histidine Biosynthesis in Yeast. Basic Life Sci., 2, 85-95, doi: 10.1007/978-1-4684-2880-3_7.

Stovicek, V., Borodina, I., and Forster, J. (2015). CRISPR—Cas system enables fast and simple genome editing of industrial Saccharomyces cerevisiae strains. Metab. Eng. Commun. 2, 13-22. doi:10.1016/j.meteno.2015.03.001.

Items
1. A yeast cell capable of producing ergothioneine, said yeast cell expressing:
   a) at least one first heterologous enzyme capable of converting L-histidine and/or L-cysteine to S-(hercyn-2-yl)-L-cysteine-S-oxide; and
   b) at least one second heterologous enzyme capable of converting S-(hercyn-2-yl)-L-cysteine-S-oxide to 2-(hydroxysulfanyl)-hercynine;
   wherein the yeast cell is further capable of converting 2-(hydroxysulfanyl)-hercynine to ergothioneine.
2. The yeast cell according to item 1, wherein the yeast cell is a GRAS organism.
3. The yeast cell according to any one of the previous items, wherein the yeast cell comprises at least two copies of the gene encoding the first heterologous enzyme.
4. The yeast cell according to any one of the previous items, wherein the yeast cell comprises at least two copies of the second heterologous enzyme.
5. The yeast cell according to any one of the previous items, wherein the yeast cell is capable of producing at least 100 mg/L, such as at least 150 mg/L, such as at least 200 mg/L, such as at least 250 mg/L histidine.
6. The yeast cell according to any one of the previous items, wherein the yeast cell further expresses or overexpresses one or more of the following:
   a. a ergothioneine transporter, such as MsErgT (SEQ ID NO:35) or variants thereof having at least 70% homology thereto;
   b. a ergothioneine transporter, such as HsSLC22A4 (SEQ ID NO:36) or variants thereof having at least 70% homology thereto;
   c. a ergothioneine transporter, such as AtOCT1 (SEQ ID NO:37) or variants thereof having at least 70% homology thereto;
   d. a ergothioneine transporter, such as ScAQR1 (SEQ ID NO:39) or variants thereof having at least 70% homology thereto;
   e. a ergothioneine transporter, such as HsSLC22A16 (SEQ ID NO:41) or variants thereof having at least 70% homology thereto;
   f. a ergothioneine transporter, such as HsSLC22A32 (SEQ ID NO:43) or variants thereof having at least 70% homology thereto;
   g. an adenylyl-sulfate kinase, such as ScMET14 (SEQ ID NO: 47) or variants thereof having at least 70% homology thereto;
   h. a phosphoadenosine phosphosulphate reductase, such as ScMET16 (SEQ ID NO: 49) or variants thereof having at least 70% homology thereto; and/or
   i. a transcription factor for nitrogenous compound transporters, such as STP1 (SEQ ID NO: 45) or variants thereof having at least 70% homology thereto.
7. The yeast cell according to any one of the previous items, wherein the yeast cell further comprises one or more mutation(s) in one or more of the following gene(s)
   a. ScAGP2;
   b. ScTPO4;
   c. ScTPO3;
   d. ScTPO1;
   e. ScURE2;
   f. ScSTR2;
   g. ScERG4;
   h. ScSPE2; and/or
   i. ScGCN4, such as one or more mutation(s) in the upstream start codons of GCN4.
8. The yeast cell according to any one of the preceding items, wherein the yeast cell does not natively produce ergothioneine.
9. The yeast cell according to any one of the preceding items, wherein the genus of said yeast cell is selected from the group consisting of Saccharomyces, Pichia, Yarrowia, Kluyveromyces, Candida, Rhodotorula, Rhodosporidium, Cryptococcus, Schizosaccharomyces, Trichosporon and Lipomyces, preferably the genus is Saccharomyces, Pichia, Yarrowia, or Kluyveromyces.
10. The yeast cell according to any one of the preceding items, wherein the yeast is selected from the group consisting of Saccharomyces cerevisiae, Pichia pastoris, Komagataella phaffii, Kluyveromyces marxianus, Kluyveromyces lactis, Schizosaccharomyces pombe, Cryptococcus albidus, Lipomyces lipofera, Lipomyces starkeyi, Rhodosporidium toruloides, Rhodotorula glutinis, Trichosporon pullulan and Yarrowia lipolytica, preferably the yeast is Saccharomyces cerevisiae, Kluyveromyces marxianus or Yarrowia lipolytica.
11. The yeast cell according to any one of the preceding items, wherein the first heterologous enzyme has an EC number selected from EC 2.1.1.44, EC 1.14.99.51, EC 6.3.2.2, EC 1.14.99.50 and EC 3.5.1.118, preferably the EC number is EC 2.1.1.44 or EC 1.14.99.51.
12. The yeast cell according to any one of the preceding items, wherein the first heterologous enzyme is an enzyme derived from a eukaryote, such as a fungus.
13. The yeast cell according to any one of the preceding items, wherein the second heterologous enzyme is an enzyme derived from a prokaryote or a eukaryote, preferably a prokaryote.
14. The yeast cell according to any one of the preceding items, wherein the second heterologous enzyme is a β-lyase or a hercynylcysteine sulfoxide lyase (EC 4.4.1.-).
15. The yeast cell according to any one of the preceding items, wherein the first heterologous enzyme is Egt1 from Neurospora crassa, Claviceps purpurea, Schizosaccharomyces pombe, Rhizopus stolonifera, Aspergillus nidulans, Aspergillus niger, Penicillium roqueforti, Penicillium notatum, Sporobolomyces salmonicolor, Aspergillus oryzae, Aspergillus carbonarius, Neurospora tetrasperma, Agaricus bisporus, Pleurotus ostreatus, Lentinula edodes or Grifola frondosa, or a functional variant thereof having at least 70% homology thereto.
16. The yeast cell according to any one of the preceding items, wherein the first heterologous enzyme is selected from the group consisting of: NcEgt1 (SEQ ID NO: 2), SpEgt1 (SEQ ID NO: 4) and CpEgt1 (SEQ ID NO: 6), and functional variants thereof having at least 70% homology to SEQ ID NO: 2, SEQ ID NO: 4 or SEQ ID NO: 6.
17. The yeast cell according to any one of the preceding items, wherein the second heterologous enzyme is:
   Egt2 from Neurospora crassa, Claviceps purpurea, Schizosaccharomyces pombe, Rhizopus stolonifera, Aspergillus nidulans, Aspergillus niger, Penicillium roqueforti, Penicillium notatum, Sporobolomyces salmonicolor, Aspergillus oryzae, Aspergillus carbonarius, Neurospora tetrasperma, Agaricus bisporus, Pleurotus ostreatus, Lentinula edodes, Grifola frondosa, Ganoderma lucidum, Cantharellus cibarius, or
   EgtE from Mycobacterium smegmatis, Nocardia asteroids, Streptomyces albus, Streptomyces fradiae, Streptomyces griseus, Actinoplanes philippinensis, Aspergillus fumigatus, Mycobacterium tuberculosis, Mycobacterium kansasii, Mycobacterium intracellulare, Mycobacterium fortuitum, Mycobacterium ulcerans, Mycobacterium balnei, Mycobacterium leprae, Mycobacterium avium, Mycobacterium bovis, Mycobacterium marinum, Mycobacterium microti, Mycobacterium paratuberculosis, Mycobacterium phlei, Rhodococcus rhodocrous (Mycobacterium rhodocrous), Arthrospira platensis, Arthrospira maxima, Aphanizomenon flos-aquae, Scytonema sp., Oscillatoria sp. and Rhodophyta sp.;
   or functional variants thereof having at least 70% homology thereto.
18. The yeast cell according to any one of the preceding items, wherein the second heterologous enzyme is selected from the group consisting of: NcEgt2 (SEQ ID NO: 8), SpEgt2 (SEQ ID NO: 10), CpEgt2 (SEQ ID NO: 12), and MsEgtE (SEQ ID NO: 14), or variants thereof having at least 70% homology to SEQ ID NO: 8, SEQ ID NO: 10, SEQ ID NO: 12 or SEQ ID NO: 14.
19. The yeast cell according to any one of the preceding items, wherein the first and the second heterologous enzymes are:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2;
xii) CpEgt1 and MsEgtE,
or functional variants thereof having at least 70% homology thereto.
20. The yeast cell according to any one of the preceding items, wherein the first and the second heterologous enzymes are:
i) NcEgt1 and CpEgt2;
ii) NcEgt1 and SpEgt2;
iii) NcEgt1 and NcEgt2;
iv) NcEgt1 and MsEgtE;
xii) CpEgt1 and MsEgtE,
or functional variants thereof having at least 70% homology thereto.
21. The yeast cell according to any one of the preceding items, wherein the first and the second heterologous enzymes are not:
iii) NcEgt1 and NcEgt2; or
viii) SpEgt1 and MsEgtE; or
x) CpEgt1 and SpEgt2.
22. The yeast cell according to any one of the preceding items, wherein the yeast cell further expresses or overexpresses an ergothioneine transporter, optionally a heterologous ergothioneine transporter, such as MsErgT (SEQ ID NO: 35) or HsSLC22A4 (SEQ ID NO: 36) or variants thereof having at least 70% homology thereto.
23. The yeast cell according to any one of the preceding items, wherein the yeast cell is capable of secreting at least part of the ergothioneine.
24. The yeast cell according to any one of the preceding items, wherein the yeast cell expresses or overexpresses an ergothioneine transporter such as AtOCT1 as set forth in SEQ ID NO: 37, ScAQR1 as set forth in SEQ ID NO: 39, HsSLC22A16 as set forth in SEQ ID NO: 41 or HsSLC22A32 as set forth in SEQ ID NO: 43 or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,
   or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.
25. The yeast cell according to any one of the preceding items, wherein the yeast cell carries a deletion of a gene encoding an ergothioneine transporter of S. cerevisiae such as ScAGP2, ScTPO3, ScTPO4, and/or ScTPO1 or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and a first and a second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,
   or functional variants thereof having at least 70% homology thereto.
26. The yeast cell according to any one of the preceding items, wherein the yeast cell expresses a transcription factor for nitrogenous compound transporters, such as ScSTP1 as set forth in SED ID NO: 45 or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,
   or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.
27. The yeast cell according to any one of the preceding items, wherein the yeast cell carries a deletion of the upstream start codons and/or the leader sequence of ScGCN4, or a deletion of the upstream start codons and/or the leader sequence of a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,
   or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.
28. The yeast cell according to any one of the preceding items, wherein the yeast cell carries a deletion of a gene encoding a transcriptional activator, such as ScURE2, or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,
   or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.
29. The yeast cell according to any one of the preceding items, wherein the yeast cell carries a deletion of a gene encoding a cystathionine gamma-synthase of cysteine biosynthesis, such as ScSTR2, or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
xiii) NcEgt1 and CpEgt2;
xiv) NcEgt1 and SpEgt2;
xv) NcEgt1 and NcEgt2;
xvi) NcEgt1 and MsEgtE;
xvii) SpEgt1 and NcEgt2;
xviii) SpEgt1 and SpEgt2;
xix) SpEgt1 and CpEgt2;
xx) SpEgt1 and MsEgtE;
xxi) CpEgt1 and NcEgt2;
xxii) CpEgt1 and SpEgt2;
xxiii) CpEgt1 and CpEgt2; and
xxiv) CpEgt1 and MsEgtE,
   or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.
30. The yeast cell according to any one of the preceding items, wherein the yeast cell carries one or more mutations in one or more genes encoding histidine, such as ScHIS1, or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,
   or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.
31. The yeast cell according to any one of the preceding items, wherein the yeast cell carries a deletion of a gene encoding a S-adenosylmethionine decarboxylase and/or delta(24(24(1)))-sterol reductase in S-adenosylmethionine (SAM) biosynthesis, such as ScSPE2 and/or ScERG4, or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,
   or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.
32. The yeast cell according to any one of the preceding items, wherein the yeast cell further expresses or overexpresses an adenylyl-sulfate kinase (ScMET14) as set forth in SEQ ID NO: 47, or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,
   or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.
33. The yeast cell according to any one of the preceding items, wherein the yeast cell expresses or overexpresses a phosphoadenosine phosphosulfate reductase (ScMET16) as set forth in SEQ ID NO:49 or a functional homologue thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto, and expresses at least one first and at least one second heterologous enzymes selected from the group consisting of:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2; and
xii) CpEgt1 and MsEgtE,
   or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.
34. The yeast cell according to any one of the preceding items, wherein the yeast cell is capable of producing ergothioneine with a total titer of at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L, such as at least 11 mg/L, such as at least 12 mg/L, such as at least 13 mg/L, such as at least 14 mg/L, such as at least 15 mg/L, such as at least 20 mg/L, such as at least 25 mg/L, such as at least 30 mg/L, such as at least 35 mg/L, such as at least 40 mg/L, such as at least 45 mg/L, such as at least 50 mg/L, such as at least 100 mg/L, such as at least 150 mg/L, such as at least 200 mg/L, such as at least 300 mg/L, such as at least 400 mg/L, such as at least 500 mg/L, such as at least 600 mg/L, such as at least 700 mg/L, such as at least 800 mg/L, such as at least 900 mg/L, such as at least 1 g/L, or more, wherein the total titer is the sum of the intracellular ergothioneine titer and the extracellular ergothioneine titer.
35. The yeast cell according to any one of the preceding items, wherein the yeast cell is capable of producing extracellular ergothioneine with a titer of at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L, such as at least 11 mg/L, such as at least 12 mg/L, such as at least 13 mg/L, such as at least 14 mg/L, such as at least 15 mg/L, such as at least 20 mg/L, such as at least 25 mg/L, such as at least 30 mg/L, such as at least 35 mg/L, such as at least 40 mg/L, such as at least 45 mg/L, such as at least 50 mg/L, such as at least 100 mg/L, such as at least 150 mg/L, such as at least 200 mg/L, such as at least 300 mg/L, such as at least 400 mg/L, such as at least 500 mg/L, such as at least 600 mg/L, such as at least 700 mg/L, such as at least 800 mg/L, such as at least 900 mg/L, such as at least 1 g/L, or more.
36. The yeast cell according to any one of the preceding items, wherein the yeast cell is capable of producing intracellular ergothioneine with a titer of at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L, such as at least 11 mg/L, such as at least 12 mg/L, such as at least 13 mg/L, such as at least 14 mg/L, such as at least 15 mg/L, such as at least 20 mg/L, such as at least 25 mg/L, such as at least 30 mg/L, such as at least 35 mg/L, such as at least 40 mg/L, such as at least 45 mg/L, such as at least 50 mg/L, such as at least 100 mg/L, such as at least 150 mg/L, such as at least 200 mg/L, such as at least 300 mg/L, such as at least 400 mg/L, such as at least 500 mg/L, such as at least 600 mg/L, such as at least 700 mg/L, such as at least 800 mg/L, such as at least 900 mg/L, such as at least 1 g/L, or more.
37. The yeast cell according to any one of the preceding items, wherein the yeast cell is capable of synthesising L-histidine and/or L-cysteine.
38. A method of producing ergothioneine in a yeast cell, comprising the steps of:
i) providing a yeast cell capable of producing ergothioneine, said yeast cell expressing:
   a) at least one first heterologous enzyme capable of converting L-histidine and/or L-cysteine to S-(hercyn-2-yl)-L-cysteine-S-oxide; and
   b) at least one second heterologous enzyme capable of converting S-(hercyn-2-yl)-L-cysteine-S-oxide to 2-(hydroxysulfanyl)-hercynine;
      wherein the yeast cell is further capable of converting 2-(hydroxysulfanyl)-hercynine to ergothioneine;
ii) incubating said yeast cell in a medium;
   thereby obtaining ergothioneine.
39. The method according to item 38, wherein the yeast cell is as defined in any one of items 1 to 37.
40. The method according to any one of items 38 to 39, wherein ergothioneine is obtained with a total titer of at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L, such as at least 11 mg/L, such as at least 12 mg/L, such as at least 13 mg/L, such as at least 14 mg/L, such as at least 15 mg/L, such as at least 20 mg/L, such as at least 25 mg/L, such as at least 30 mg/L, such as at least 35 mg/L, such as at least 40 mg/L, such as at least 45 mg/L, such as at least 50 mg/L, such as at least 100 mg/L, such as at least 150 mg/L, such as at least 200 mg/L, such as at least 300 mg/L, such as at least 400 mg/L, such as at least 500 mg/L, such as at least 600 mg/L, such as at least 700 mg/L, such as at least 800 mg/L, such as at least 900 mg/L, such as at least 1 g/L, or more, wherein the total titer is the sum of the intracellular ergothioneine titer and the extracellular ergothioneine titer.
41. The method according to any one of items 38 to 40, wherein the yeast cell is a

GRAS organism.
42. The method according to any one of items 38 to 41, wherein the yeast cell does not natively produce ergothioneine.
43. The method according to any one of items 38 to 42, wherein the genus of said yeast cell is selected from the group consisting of Saccharomyces, Pichia, Yarrowia, Kluyveromyces, Candida, Rhodotorula, Rhodosporidium, Cryptococcus, Trichosporon and Lipomyces.
44. The method according to any one of items 38 to 43, wherein the yeast is selected from the group consisting of Saccharomyces cerevisiae, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica.
45. The method according to any one of items 38 to 44, wherein the yeast cell comprises a first nucleic acid encoding the first heterologous enzyme and/or a second nucleic acid encoding the second heterologous enzyme.
46. The method according to any one of items 38 to 45, wherein the first nucleic acid is comprised within the genome of the yeast cell or on a vector comprised within the yeast cell.
47. The method according to any one of items 38 to 46, wherein the second nucleic acid is comprised within the genome of the yeast cell or on a vector comprised within the yeast cell.
48. The method according to any one of items 38 to 47, wherein the first and/or the second nucleic acids are present in high copy number.
49. The method according to any one of items 38 to 48, wherein the first and/or the second nucleic acids are under the control of an inducible promoter.
50. The method according to any one of items 38 to 49, wherein the first and/or the second nucleic acids are codon-optimised for expression in the yeast cell.
51. The method according to any one of items 38 to 50, wherein the yeast cell is capable of secreting ergothioneine into the medium.
52. The method according to any one of items 38 to 51, wherein the medium comprises at least one amino acid such as histidine, preferably L-histidine, cysteine, preferably L-cysteine, or methionine, preferably L-methionine, preferably at a concentration of at least 0.1 g/L, such as at least 0.2 g/L, such as at least 0.3 g/L, such as at least 0.4 g/L, such as at least 0.5 g/L, such as at least 0.75 g/L, such as at least 1 g/L, such as at least 2 g/L.
53. The method according to any one of items 38 to 52, further comprising the step of recovering the ergothioneine from the medium.
54. The method according to any one of items 38 to 52, wherein the yeast cell is capable of synthesising L-histidine and/or L-cysteine.
55. A polypeptide having the sequence as set forth in SEQ ID NO: 6 (CpEgt1) or a variant thereof having at least 70% homology to SEQ ID NO: 6.
56. A polypeptide having the sequence as set forth in SEQ ID NO: 12 (CpEgt2) or a variant thereof having at least 70% homology to SEQ ID NO: 12.
57. A nucleic acid encoding the polypeptide of item 55 and/or the polypeptide of item 56.
58. The nucleic acid according to item 57, codon-optimised for expression in a yeast cell such as Saccharomyces cerevisiae or Yarrowia lipolytica.
59. The nucleic acid according to any one of items 57 to 58, having the sequence as set forth in SEQ ID NO: 7 or SEQ ID NO: 17, or having at least 70% homology to SEQ ID NO: 7 or SEQ ID NO: 17.
60. The nucleic acid according to any one of items 57 to 58, having the sequence as set forth in SEQ ID NO: 5 or SEQ ID NO: 16, or having at least 70% homology to SEQ ID NO: 5 or SEQ ID NO: 16.
61. The nucleic acid according to any one of items 57 to 58, having the sequence as set forth in SEQ ID NO: 11 or SEQ ID NO: 18, or having at least 70% homology to SEQ ID NO: 11 or SEQ ID NO: 18.
62. A vector comprising a nucleic acid sequence as defined in any one of items 57 to 58.
63. A host cell expressing at least one of the polypeptides according to any one of items 55 or 56 or comprising the nucleic acid according to any one of items 57 to 61 or the vector according to item 62.
64. The host cell according to item 63, expressing the polypeptides of items 55 and 56.
65. Use of the polypeptide of any one of items 55 or 56, of the nucleic acid of any one of items 57 to 61, of the host cell of any one of items 63 to 64, or of the vector of item 62, for the production of ergothioneine.
66. Ergothioneine obtained by the method according to any one of items 38 to 54.

1. A yeast cell capable of producing ergothioneine, said yeast cell expressing:
a) at least one first heterologous enzyme capable of converting L-histidine and/or L-cysteine to S-(hercyn-2-yl)-L-cysteine-S-oxide; and
b) at least one second heterologous enzyme capable of converting S-(hercyn-2-yl)-L-cysteine-S-oxide to 2-(hydroxysulfanyl)-hercynine;
wherein the yeast cell is further capable of converting 2-(hydroxysulfanyl)-hercynine to ergothioneine.

2. The yeast cell according to claim 1, wherein the yeast is selected from the group consisting of Saccharomyces cerevisiae, Pichia pastoris, Komagataella phaffii, Kluyveromyces marxianus, Kluyveromyces lactis, Schizosaccharomyces pombe, Cryptococcus albidus, Lipomyces lipofera, Lipomyces starkeyi, Rhodosporidium toruloides, Rhodotorula glutinis, Trichosporon pullulan and Yarrowia lipolytica, preferably the yeast is Saccharomyces cerevisiae, Kluyveromyces marxianus or Yarrowia lipolytica.

3. The yeast cell according to any one of the preceding claims, wherein the first and the second heterologous enzymes are:
i) NcEgt1 and CpEgt2;
ii) NcEgt1 and SpEgt2;
iii) NcEgt1 and NcEgt2;
iv) NcEgt1 and MsEgtE;
v) SpEgt1 and NcEgt2;
vi) SpEgt1 and SpEgt2;
vii) SpEgt1 and CpEgt2;
viii) SpEgt1 and MsEgtE;
ix) CpEgt1 and NcEgt2;
x) CpEgt1 and SpEgt2;
xi) CpEgt1 and CpEgt2;
xii) CpEgt1 and MsEgtE,
or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

4. The yeast cell according to any one of the preceding claims, wherein the first and the second heterologous enzymes are:
i) NcEgt1 and CpEgt2;
ii) NcEgt1 and SpEgt2;
iii) NcEgt1 and NcEgt2;
iv) NcEgt1 and MsEgtE;
xii) CpEgt1 and MsEgtE,
or functional variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

5. The yeast cell according to any one of the preceding claims, wherein the yeast cell further expresses or overexpresses an ergothioneine transporter, optionally a heterologous ergothioneine transporter, such as MsErgT (SEQ ID NO: 35) or HsSLC22A4 (SEQ ID NO: 36) or variants thereof having at least 70% homology thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

6. The yeast cell according to any one of the preceding claims, wherein the yeast cell is capable of producing ergothioneine with a total titer of at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L, such as at least 11 mg/L, such as at least 12 mg/L, such as at least 13 mg/L, such as at least 14 mg/L, such as at least 15 mg/L, such as at least 20 mg/L, such as at least 25 mg/L, such as at least 30 mg/L, such as at least 35 mg/L, such as at least 40 mg/L, such as at least 45 mg/L, such as at least 50 mg/L, such as at least 100 mg/L, such as at least 150 mg/L, such as at least 200 mg/L, such as at least 300 mg/L, such as at least 400 mg/L, such as at least 500 mg/L, such as at least 600 mg/L, such as at least 700 mg/L, such as at least 800 mg/L, such as at least 900 mg/L, such as at least 1 g/L, or more, wherein the total titer is the sum of the intracellular ergothioneine titer and the extracellular ergothioneine titer.

7. A method of producing ergothioneine in a yeast cell, comprising the steps of:
i) providing a yeast cell capable of producing ergothioneine, said yeast cell expressing:
a) at least one first heterologous enzyme capable of converting L-histidine and/or L-cysteine to S-(hercyn-2-yl)-L-cysteine-S-oxide; and
b) at least one second heterologous enzyme capable of converting S-(hercyn-2-yl)-L-cysteine-S-oxide to 2-(hydroxysulfanyl)-hercynine;
wherein the yeast cell is further capable of converting 2-(hydroxysulfanyl)-hercynine to ergothioneine;
ii) incubating said yeast cell in a medium;
thereby obtaining ergothioneine, wherein optionally the yeast cell is a GRAS organism.

8. The method according to claim 7, wherein the yeast cell comprises a first nucleic acid encoding the first heterologous enzyme and/or a second nucleic acid encoding the second heterologous enzyme.

9. The method according to any one of claims 7 to 8, wherein the medium comprises at least one amino acid such as histidine, preferably L-histidine, cysteine, preferably L-cysteine, or methionine, preferably L-methionine, preferably at a concentration of at least 0.1 g/L, such as at least 0.2 g/L, such as at least 0.3 g/L, such as at least 0.4 g/L, such as at least 0.5 g/L, such as at least 0.75 g/L, such as at least 1 g/L, such as at least 2 g/L.

10. A polypeptide having the sequence as set forth in SEQ ID NO: 6 (CpEgt1) or a variant thereof having at least 70% homology to SEQ ID NO: 6.

11. A polypeptide having the sequence as set forth in SEQ ID NO: 12 (CpEgt2) or a variant thereof having at least 70% homology to SEQ ID NO: 12.

12. A nucleic acid encoding the polypeptide of claim 10 and/or the polypeptide of claim 11, optionally wherein the nucleic acid is codon-optimised for expression in a yeast cell such as Saccharomyces cerevisiae or Yarrowia lipolytica and/or optionally wherein the nucleic acid comprises or consists of the sequence as set forth in SEQ ID NO: 7, SEQ ID NO: 17, SEQ ID NO: 5, SEQ ID NO: 16, SEQ ID NO: 11 or SEQ ID NO: 18, or comprises or consists of a sequence having at least 70% homology thereto.

13. A vector comprising a nucleic acid sequence as defined in claim 12.

14. A host cell expressing at least one of the polypeptides according to any one of claim 10 or 11 or comprising the nucleic acid according to claim 12 or the vector according to claim 13.

15. Use of the polypeptide of any one of claim 10 or 11, of the nucleic acid of claim 12, of the host cell of claim 13, or of the vector of claim 14, for the production of ergothioneine.


SEQUENCE LISTING (51 sequences)

SEQ ID NO: 1 - DNA, 2631 bp, Neurospora crassa
atgccgagtgccgaatccatgaccccaagcagtgccctcggacagctcaaagcaactggacaacatgtgctatccaagcttcagcagcagacatcaaacgccgatatcatcgacatccgccgcgttgctgtagagatcaacctcaagaccgagataacctccatgttccgacctaaagatggccctagacagctacccaccttgcttctctacaacgagagaggcctgcagctgttcgagcgtatcacataccttgaagagtactatcttaccaatgacgagatcaaaatcctcaccaaacatgcgaccgaaatggctagcttcatcccgtcaggtgccatgatcattgagctcggaagcggaaatctgcgcaaagtaaaccttctattggaagccctagacaacgccggcaaggcaattgactattatgcccttgacctgtctcgggaggagctggagcgcactctcgctcaggtaccatcctacaagcacgtcaagtgccacggtcttctgggtacatatgacgatggacgtgactggctcaaggccccagagaacatcaataaacagaaatgcatcttgcacctcgggtcaagcattggcaactttaaccgcagtgacgccgctacctttctcaagggctttacggacgtccttggacccaatgacaagatgctcattggggttgacgcttgcaatgacccggcgagggtataccacgcttacaacgacaaggttggtattactcacgagttcatcttgaatggtcttcgcaacgccaatgaaattatcggagagacggccttcatcgagggcgattggagagtcattggcgaatatgtgtatgacgaagagggcggcagacaccaggccttttacgcccccactcgcgacaccatggttatgggggagttgattaggtcacacgacaggatccagatcgaacagagcctaaagtactcgaaagaggagtcagagaggctctggagcacggcgggattggaacaagtctcggaatggacgtacggcaacgaatatggactccatctgcttgccaagtcaaggatgtctttcagtctcatcccttcggtgtacgctcgcagcgcactcccaactctggacgactgggaggccctttgggcgacatgggatgtcgtcacacgtcagatgcttccccaggaagagcttctggagaagcccatcaagctccgaaacgcctgcatcttttacctcggtcacatcccgaccttcctcgacatccagctcacaaagaccaccaagcaggctccgtcagagcccgctcacttttgcaagatcttcgagcgaggcattgatcctgatgtcgacaacccggagctgtgtcatgcgcactcggagattcctgatgaatggccgccggtggaagaaatcctgacctaccaggagacggtacggtcccggttacgcggcctctatgcgcatggcatcgcgaatattccgcggaatgtgggtcgggccatttgggttgggtttgagcacgagcttatgcatatcgagacgctgttgtacatgatgctacagagcgacaagacgctgatcccaacccatattccacggcccgactttgacaagctcgcgaggaaggcagagtccgagagggttcccaatcagtggtttaagattccggcacaggagatcaccatcggtttggatgatcctgaggatggatctgatatcaacaagcattatggctgggacaacgagaagcctccaaggcgcgttcaagttgctgcctttcaggctcaagggaggccgatcaccaacgaagagtacgcgcaatatctgcttgaaaagaacatcgacaagctccctgcctcttgggcccgcctggacaacgagaacattagcaatggaacaacaaacagcgtgagcggtcaccacagcaacagaacctccaagcagcagctcccttcatctttcctcgagaagacagcagtccgcacagtctacggtctcgtgcctctcaagcacgctctcgactggcccgtgtttgcctcttacgacgaacttgccggttgcgcagcttacatgggcggccgtattcccaccttcgaagagacccggagcatttacgcttacgccgatgctctcaagaagaagaaggaagctgagagacaattgggaaggacggttccggctgttaatgcccacctaaccaacaacggcgtggaaatcactcccccatcctctccctcttccgagacccccgccgagtcttcctccccctccgacagcaacaccaccctcatcaccaccgaagacctcttctctgacctagacggtgccaatgtcggttttcacaactggcaccctatgcccatcacctccaaaggcaacacccttgtcgggcaaggcgagctcggcggcgtgtgggaatggacttcatcggtcctccgcaagtgggaggggttcgagccgatggagctgtaccccggctatacggcggattttttcgatgagaagcacaacattgtgctgggagggagctgggctacgcatccgaggattgcggggaggaagagctttgtgaattggtaccagaggaattatccttatgcttgggtgggggcgagagttgttagggatttgtga

SEQ ID NO: 2 - protein, 876 aa, Neurospora crassa
MPSAESMTPSSALGQLKATGQHVLSKLQQQTSNADIIDIRRVAVEINLKTEITSMFRPKDGPRQLPTLLLYNERGLQLFERITYLEEYYLTNDEIKILTKHATEMASFIPSGAMIIELGSGNLRKVNLLLEALDNAGKAIDYYALDLSREELERTLAQVPSYKHVKCHGLLGTYDDGRDWLKAPENINKQKCILHLGSSIGNFNRSDAATFLKGFTDVLGPNDKMLIGVDACNDPARVYHAYNDKVGITHEFILNGLRNANEIIGETAFIEGDWRVIGEYVYDEEGGRHQAFYAPTRDTMVMGELIRSHDRIQIEQSLKYSKEESERLWSTAGLEQVSEWTYGNEYGLHLLAKSRMSFSLIPSVYARSALPTLDDWEALWATWDVVTRQMLPQEELLEKPIKLRNACIFYLGHIPTFLDIQLTKTTKQAPSEPAHFCKIFERGIDPDVDNPELCHAHSEIPDEWPPVEEILTYQETVRSRLRGLYAHGIANIPRNVGRAIWVGFEHELMHIETLLYMMLQSDKTLIPTHIPRPDFDKLARKAESERVPNQWFKIPAQEITIGLDDPEDGSDINKHYGWDNEKPPRRVQVAAFQAQGRPITNEEYAQYLLEKNIDKLPASWARLDNENISNGTTNSVSGHHSNRTSKQQLPSSFLEKTAVRTVYGLVPLKHALDWPVFASYDELAGCAAYMGGRIPTFEETRSIYAYADALKKKKEAERQLGRTVPAVNAHLTNNGVEITPPSSPSSETPAESSSPSDSNTTLITTEDLFSDLDGANVGFHNWHPMPITSKGNTLVGQGELGGVWEWTSSVLRKWEGFEPMELYPGYTADFFDEKHNIVLGGSWATHPRIAGRKSFVNWYQRNYPYAWVGARVVRDL

SEQ ID NO: 3 - DNA, 2322 bp, Schizosaccharomyces pombe
atgacagaaatagaaaacattggcgcattagaagttctcttctctcctgaatccatcgagcagagcctcaaacggtgtcaactcccctccactttattatacgatgaaaaaggtttacgactgtttgatgagattacgaatttaaaagaatactacctgtatgaaagtgagcttgatattctgaagaagttcagcgattccattgccaaccagttactgtctccagatcttcctaacacggttatagaattagggtgtggaaatatgcgcaaaacaaaacttcttttagatgcgtttgaaaagaagggctgtgatgtgcatttttacgcccttgaccttaatgaagccgagttgcaaaaaggactgcaggagcttcgtcagactaccaattatcagcatgttaaggtgtctggtatttgcggttgctttgaaagattgctacaatgtttggacaggtttcgtagtgagcccaatagtcgaattagcatgttgtacttgggtgcttcgattggtaattttgataggaaatccgcagcatcatttttacgttcgtttgccagtcgtttgaatattcatgacaaccttttaatctccttcgatcatagaaacaaggctgagctagtccaactagcttacgatgatccttatcgtattactgaaaaatttgaaaagaatattttggctagtgtcaatgcggtttttggtgaaaaccttttcgacgaaaatgattgggaatataaaagtgtctacgatgaagatctcggtgttcatagggcctacttacaagccaaaaatgaagttactgttattaagggtccaatgttttttcaatttaaacctagtcatttaattttgatcgaagaaagttggaagaatagcgatcaagaatgtcgtcaaatcattgagaaaggtgattttaaattagtctctaagtatgaaagtacgattgcagattactcgacctatgttattaccaaacaatttcctgctatgcttcaactccctcttcagccttgtccttcgttagcagaatgggatgctctacgcaaagtatggctttttattacaaataaattgcttaacaaagataacatgtacaccgcatggattcctttgagacaccctccaattttttacatcggacatgtccctgtttttaatgatatttatctcacaaagattgtcaaaaacaaagcaactgctaacaaaaaacatttttgggagtggtttcaacgtggtatagatccggacattgaagatccctccaagtgccattggcattctgaagttcctgaaagctggccttctcctgaccaacttcgtgaatacgagaaagagtcttgggaatatcatattgtaaagttgtgcaaagcaatggatgaattgtctacttctgaaaagagaattctctggctttgttacgaacatgtagccatgcatgtggagacaactctttacatctacgtacagtcatttcaaaatgcaaaccagactgtatcaatttgcggatcacttcctgaaccagctgaaaaacttacgaaagctccgttatgggtgaatgtacctgaaacggaaattgcagttggtatgcccttgacaacacaatacacgagtgttggatcaaatttgcaatcatccgatcttagtgcccatgaaaatacagatgaacttttttattttgcgtgggataatgagaaaccaatgaggaagaaactggtttctagcttttctattgccaatcgtccaatttctaacggtgaatatttagattttatcaataaaaagtcaaaaacagaaagggtgtatccaaagcaatgggcggagattgatggaacgctttacatacgaaccatgtacggcttattaccccttgacgactacttgggttggcctgttatgacttcatacgacgatctaaacaattatgcgagctcccaaggatgcagactaccaactgaggatgaactgaactgtttttacgatcgggttctcgagagaactgatgagccttatgttagtaccgaaggaaaggcaactggttttcaacaattgcaccctttagccttaagtgataattcaagtaatcaaatattcacaggagcatgggaatggacaagtacagttctggagaagcacgaggattttgaacctgaagagctttatccagattatacacgagatttctttgatggaaagcataatgtcgttttgggtggtagctttgctacggctacgcgcatttcaaatagaagaagcttcaggaacttttaccaagctggctataaatatgcatggattggagctagactagtcaaaaactaa

SEQ ID NO: 4 - protein, 773 aa, Schizosaccharomyces pombe
MTEIENIGALEVLFSPESIEQSLKRCQLPSTLLYDEKGLRLFDEITNLKEYYLYESELDILKKFSDSIANQLLSPDLPNTVIELGCGNMRKTKLLLDAFEKKGCDVHFYALDLNEAELQKGLQELRQTTNYQHVKVSGICGCFERLLQCLDRFRSEPNSRISMLYLGASIGNFDRKSAASFLRSFASRLNIHDNLLISFDHRNKAELVQLAYDDPYRITEKFEKNILASVNAVFGENLFDENDWEYKSVYDEDLGVHRAYLQAKNEVTVIKGPMFFQFKPSHLILIEESWKNSDQECRQIIEKGDFKLVSKYESTIADYSTYVITKQFPAMLQLPLQPCPSLAEWDALRKVWLFITNKLLNKDNMYTAWIPLRHPPIFYIGHVPVFNDIYLTKIVKNKATANKKHFWEWFQRGIDPDIEDPSKCHWHSEVPESWPSPDQLREYEKESWEYHIVKLCKAMDELSTSEKRILWLCYEHVAMHVETTLYIYVQSFQNANQTVSICGSLPEPAEKLTKAPLWVNVPETEIAVGMPLTTQYTSVGSNLQSSDLSAHENTDELFYFAWDNEKPMRKKLVSSFSIANRPISNGEYLDFINKKSKTERVYPKQWAEIDGTLYIRTMYGLLPLDDYLGWPVMTSYDDLNNYASSQGCRLPTEDELNCFYDRVLERTDEPYVSTEGKATGFQQLHPLALSDNSSNQIFTGAWEWTSTVLEKHEDFEPEELYPDYTRDFFDGKHNVVLGGSFATATRISNRRSFRNFYQAGYKYAWIGARLVKN

SEQ ID NO: 5 - DNA, 2529 bp, Neurospora crassa
atgacagccgtcaagcagatccccgaaaggaaggtcctcatcgactcgaaccacaaatcaccctcgaaacccggcaaacacccaaatagtgttatcgacattcgtagcaacaaggacgatctaaacctcagacatgcacttgtatcgtcgttcaatcctcacgatggtaagccccgctggctgcctaccatgctgctatacgatgagaagggtctgcaattattcgaggatattacgtatctggacgagtattacttgactggctacgaaatcgagctgctgaagaagcactccgccgaaattgccgccgcaatccctgatggctctatggtaattgaactaggaagtggaaaccttcgaaagatttgtctccttctccaagccttcgaagattcacacaagtcgattgactactacgccctcgatctgtcgcaaaaggagctggagcgtaccctctcgcatgttcctgactttaagtacgtctcttgccatggtcttctgggcacctacgacgacggagtgacgtggttgaagcagcctggcattgtgaacaaaaccaaatgcattattcatttgggttcgagcattggaaattttcatagaaacgaggctgccgactttctgcaaaccttcgcggacgttatgaagcccgacgatagtatggtcattggcctagactcgtgtgggaatccagaaatgtcacgcatacaacgattcattctgaacggactcagcaatgccaatagcgtctacggcaaggaaatattctacgtccctgactggcgtgtcatcggcgaatacgtctacgatgacgaaggcggtcgccaccaggccttcatctctccgctcaaagaggtcaccgccctagggtctgtgatcaaagctcacgagcgtatcaagatcgagcagagtctcaaatactccaaggctagtgcggatgacctatggcgaaacgcaggttttcgcgaaactcagacctggactcggaatggcgagtatggccttcatatgctccaaagagcagacccgcccttcagcaaagctccctctctctacgccgccaatactctcccttccctctccgactggcgagccctctggtgcgcttgggatatcgtcaccagggccatgctccctcagcaggaactcaccgagaagcccatcgagcttcgacacgcatatattttctacctcggccatatcccgaccttcctcgatatacagcttacaaagacaagcgcctgggcccccactagcccagtctcctaccacgccatattcgagcgcggcatcgacccagacgttgataaccccgagaaatgccacgaccattctgaaattccagatgagtggccgcccgtggaagaaattatcgcgtaccaagatcgtgtgcgggtccgtctgaccgagctctacaagcagggcgtgcacacgattacgcgaaaagcagctagggccatctgggtcagtttcgagcatgaggcgatgcacctcgagactctattgtacatgatgctgcagagcgataaggtcttgccaccgccacacacgggtgtgcctgattttgagagaatggctacgaaggcgtttgaggctcgcacgcagaatatgtggttcgaaattcccgagcagactatttctctggggacggacgacccggaggacggggacgaagatgtgcactttggatgggacaatgaaaagccagtgcgaagagtcaaagttcatgcgcttcaggcacaaggtcggcctatcaccaatgaagagtatgccttgtacatctaccacacgaactcctctaaactacccgcctcttggtcgtcgtccccatcctcttctctttcgaatggtgtatcccaccccagcagtcacaataaacacattcccacagatcttccgcactccttcctccaaggaaagtttgtccggacagtttacggccttatccctctcagcctagctctcgactggcctgtccaagcatcttacgatgaactcgccgactgtgcgctatggatgggcggccgcatcccgactctggaagaagcccgcagcatctacgcctttgtagagtccaaaacccaaatcgccacgggaaatactctggtcaagaaagtccccgccgtgaacggccatcttgtaaacaacggcgtcgaagagacacccccccacgagtcgtcctctgcggtcgagaacagcctgttcatcgaccttgccggcctgaatgttggcttcaagagctggaatccggagccagtaacttcgagtggcacctctctggccggacagtcctctatgggaggagtttgggagtggacgtcctccgttttgcgcccccatgaaggtttccaccccatggagctgtaccctggctacacagcagacttttttgacgaaaagcacaatattgtgctgggcgggtcatgggcaactcatccaaggattgctggacgcaagagcttcgtgaattggtatcagaggaattatccatatgcgtgggctggagcgaggctggtaaaagatgcgtga

SEQ ID NO: 6 - protein, 842 aa, Claviceps purpurea
MTAVKQIPERKVLIDSNHKSPSKPGKHPNSVIDIRSNKDDLNLRHALVSSFNPHDGKPRWLPTMLLYDEKGLQLFEDITYLDEYYLTGYEIELLKKHSAEIAAAIPDGSMVIELGSGNLRKICLLLQAFEDSHKSIDYYALDLSQKELERTLSHVPDFKYVSCHGLLGTYDDGVTWLKQPGIVNKTKCIIHLGSSIGNFHRNEAADFLQTFADVMKPDDSMVIGLDSCGNPEMSRIQRFILNGLSNANSVYGKEIFYVPDWRVIGEYVYDDEGGRHQAFISPLKEVTALGSVIKAHERIKIEQSLKYSKASADDLWRNAGFRETQTWTRNGEYGLHMLQRADPPFSKAPSLYAANTLPSLSDWRALWCAWDIVTRAMLPQQELTEKPIELRHAYIFYLGHIPTFLDIQLTKTSAWAPTSPVSYHAIFERGIDPDVDNPEKCHDHSEIPDEWPPVEEIIAYQDRVRVRLTELYKQGVHTITRKAARAIWVSFEHEAMHLETLLYMMLQSDKVLPPPHTGVPDFERMATKAFEARTQNMWFEIPEQTISLGTDDPEDGDEDVHFGWDNEKPVRRVKVHALQAQGRPITNEEYALYIYHTNSSKLPASWSSSPSSSLSNGVSHPSSHNKHIPTDLPHSFLQGKFVRTVYGLIPLSLALDWPVQASYDELADCALWMGGRIPTLEEARSIYAFVESKTQIATGNTLVKKVPAVNGHLVNNGVEETPPHESSSAVENSLFIDLAGLNVGFKSWNPEPVTSSGTSLAGQSSMGGVWEWTSSVLRPHEGFHPMELYPGYTADFFDEKHNIVLGGSWATHPRIAGRKSFVNWYQRNYPYAWAGARLVKDA

SEQ ID NO: 7 - DNA, 1422 bp, Neurospora crassa
atggtcgccaccaccgtcgagctgcctctgcagcaaaaggccgacgccgcccaaactgttactggccccctcccattcggcaattccctcctcaaggaattcgtcctcgaccctgcctaccggaacctcaaccatggctccttcggcaccatcccctccgccatccaacaaaaactccgcagttaccaaaccgccgccgaagcccgcccctgccccttcctccgctaccaaacccccgtactcctcgacgaatcccgcgccgccgtcgccaacctcctcaaagtccccgtcgaaaccgtcgtcttcgtcgccaacgccactatgggcgtcaacactgtcctgcgcaacatcgtctggtccgccgacggcaaggacgagatcctctacttcgacaccatctacggcgcctgcggcaagaccatcgactacgtcatcgaagacaagcgagggatcgtttcttctcgctgtatcccattgatctaccccgccgaagacgacgatgtcgtcgctgccttccgggacgccatcaagaagagccgcgaagaaggcaagcgaccccgtctggctgttatcgacgtcgtctcctccatgcctggcgtacggttcccgttcgaggacatcgtcaagatctgcaaagaagaagagatcatctcgtgcgtggacggcgcccaaggcatcggcatggtggacctcaagatcaccgagaccgacccggattttttgattagtaactgccacaagtggctgtttactccgcgcggatgtgccgtgttctacgtgcctgtgcgtaaccagcacttgatccgctcgacgctgcctactagccatgggttcgtgccgcaggtcgggaataggttcaacccgctggtgccggcggggaacaagtcagcgtttgttagcaactttgagtttgtgggcacggtggataactcgccgttcttttgtgttaaggacgcgatcaagtggcgcgaggaggtgctcggtggggaggagaggatcatggagtacatgactaaattggcgagggaaggtggacagaaagtggcggagattctggggacgagggtgttggagaatagcacgggaacgctgatcaggtgcgccatggtcaatattgcgttgccgttcgttgtgggagaggatcccaaggcgccggtcaagttgacggagaaggaggagaaggatgttgaagggttgtatgagattccccatgaggaggcaaacatggcgttcaagtggatgtacaacgtgctgcaggacgagtttaacacgtttgtacccatgaccttccacaggaggaggttctgggccagattgagcgcgcaggtgtatttggagatgagcgatttcgagtgggcggggaagacgttgaaggagttgtgtgagagggtggctaagggggagtacaaggagagcgcctga

SEQ ID NO: 8 - protein, 473 aa, Neurospora crassa
MVATTVELPLQQKADAAQTVTGPLPFGNSLLKEFVLDPAYRNLNHGSFGTIPSAIQQKLRSYQTAAEARPCPFLRYQTPVLLDESRAAVANLLKVPVETVVFVANATMGVNTVLRNIVWSADGKDEILYFDTIYGACGKTIDYVIEDKRGIVSSRCIPLIYPAEDDDVVAAFRDAIKKSREEGKRPRLAVIDVVSSMPGVRFPFEDIVKICKEEEIISCVDGAQGIGMVDLKITETDPDFLISNCHKWLFTPRGCAVFYVPVRNQHLIRSTLPTSHGFVPQVGNRFNPLVPAGNKSAFVSNFEFVGTVDNSPFFCVKDAIKWREEVLGGEERIMEYMTKLAREGGQKVAEILGTRVLENSTGTLIRCAMVNIALPFVVGEDPKAPVKLTEKEEKDVEGLYEIPHEEANMAFKWMYNVLQDEFNTFVPMTFHRRRFWARLSAQVYLEMSDFEWAGKTLKELCERVAKGEYKESA

SEQ ID NO: 9 - DNA, 1179 bp, Schizosaccharomyces pombe
atggctgaaaacaacgtctacggccatgaaatgaaaaagcattttatgcttgatcccgattacgtgaatgtaaataacggaagttgtggaacagaatctcttgctgtttacaataaacatgtccaacttttaaaggaagctcagagcaagccagattttatgtgcaatgcctatatgccgatgtacatggaggctactcgaaatgaagttgccaagctgataggcgcggattcaagtaatatagttttttgcaattccgctacagatgggattagtacggttttgttgacatttccgtgggaacagaatgatgagatattgatgctaaatgttgcctatcctacttgtacatatgccgctgattttgcaaagaatcagcataatttacgattagacgttatcgatgttggggtggaaattgatgaagatctattccttaaagaagtagaacagcgttttttgcagtccaagccgagagcatttatctgtgatattttaagttctatgcccgttatcttgtttccttgggaaaaagtcgtaaagctttgtaaaaagtataatattgttagcattattgatggtgctcatgccataggtcatattcctatgaatttggctaatgttgatcctgattttttgtttaccaatgctcataaatggttaaactcaccagctgcatgcactgtactctatgtctcagctaaaaatcacaatctcatcgaagcacttcctctctcatacggttatggattaagagaaaaggaatcaattgccgcagatactcttaccaatcggtttgtcaattctttcaagcaagatttacctaagtttatagccgttggtgaggctattaagtttcgaaaatccattggaggggaagaaaagattcaacaatattgtcatgaaatagctttaaagggagccgaaattatttctaaagaactgggcacttcctttatcaaacctccatacccagttgcaatggtaaacgtcgaagttcccttacgcaacattccctccatagaaacacagaaagtattttggcctaaatataatacattccttcgatttatggaatttaaaggaaaattttacactagacttagcggtgcggtgtatttagaacaatcagatttctattatattgctaaagttattaaagacttctgctctctttga

SEQ ID NO: 10 - protein, 392 aa, Schizosaccharomyces pombe
MAENNVYGHEMKKHFMLDPDYVNVNNGSCGTESLAVYNKHVQLLKEAQSKPDFMCNAYMPMYMEATRNEVAKLIGADSSNIVFCNSATDGISTVLLTFPWEQNDEILMLNVAYPTCTYAADFAKNQHNLRLDVIDVGVEIDEDLFLKEVEQRFLQSKPRAFICDILSSMPVILFPWEKVVKLCKKYNIVSIIDGAHAIGHIPMNLANVDPDFLFTNAHKWLNSPAACTVLYVSAKNHNLIEALPLSYGYGLREKESIAVDTLTNRFVNSFKQDLPKFIAVGEAIKFRKSIGGEEKIQQYCHEIALKGAEIISKELGTSFIKPPYPVAMVNVEVPLRNIPSIETQKVFWPKYNTFLRFMEFKGKFYTRLSGAVYLEESDFYYIAKVIKDFCSL

SEQ ID NO: 11 - DNA, 1581 bp, Claviceps purpurea
atgggtttgttggaaggtgaagaattggttttgagaggtagaggtcaaggtggtgaacctagaccagaaagagaaccagaattgaagttggaacacgttccagaaagggctccagatggtgaaccagaaactgaaggtcaattgggtccaagaaaagaacctgaacataagttggaagctgaatccgaaccattgcaagaaactccacaaagagaagttttggcttttggtagagcttggaagtccgaatttttgtttgatccagcttggagaaacttgaaccatggtagttttggtacttaccccttgtacatcagagataagttgagagcttatcaagatcaagctgaagctagacctgatcacttcattagatacgaagagtccaagttgttgcatagatctagagctgctgttgctaagatagttaatgctccattggataccgttgttttcgttggtaatgctactgaaggtgtcaacactgtcttgagaaatttgagatgggactccttggaaaaaggtggtcaaaaggatgttatcctgtctttctctactgtttacgaagcttgtggtaacgctgctgattatatcgttgaatactttgccggtaaggttgaacatagaaccatcgaattggaatacccagttgaagatgctgatgttattgctgctttaagaggtgctgctactcaagttgctagagaaggtaaaagggctagattggctatgatggatgttgttacttctagaccaggtgttgtttttccatgggaagctgcagttagagtatgtagagaattgggtatcttgtccttggttgatggtgctcaaggtgttggtatggttagattggatttgactgctgctgatccagatttcttcgtttctaactgtcataagtggttgttggttccaagaggttgtgctatgttgtatactccagctagaactcaatgtttgttgagaactgctttggctacttctcatggttatgttccaccatctgctgctccagctccaccaggttctaaatctagatatgttgctaacttcgaattcgttggcactagagataatggtccatatttgtgtgttgctgatgcaattgcttggagagaacgtgtttgtggtggtgaagaaaacatcttgagatacttgtgggctttgaacaagaagggtattagaattgtcgctagagctttgggtactacccatttggataacgaaactgaaactttgaccaactgtgctatgggtaatgttgctttgccaatgagagttgatgatgaagatgcctctactgctttagatgctgctccttctgctgctattgctgcaccagatgttgttgttgcaagagaaaatgttgcattggttgacaagtggatgagagaaagattattcgatgactacaagaccttcatgaccttgttcgttatgcaagatagatactgggttagactgtctgctcaaatctacttggatgaacaagattatgaagccgccggtgatattttgaaagctttgtgtgaaagaatcaggcgtagagaatatttggttccacaaccagttgagtaa

SEQ ID NO: 12 - protein, 526 aa, Claviceps purpurea
MGLLEGEELVLRGRGQGGEPRPEREPELKLEHVPERAPDGEPETEGQLGPRKEPEHKLEAESEPLQETPQREVLAFGRAWKSEFLFDPAWRNLNHGSFGTYPLYIRDKLRAYQDQAEARPDHFIRYEESKLLHRSRAAVAKIVNAPLDTVVFVGNATEGVNTVLRNLRWDSLEKGGQKDVILSFSTVYEACGNAADYIVEYFAGKVEHRTIELEYPVEDADVIAALRGAATQVAREGKRARLAMMDVVTSRPGVVFPWEAAVRVCRELGILSLVDGAQGVGMVRLDLTAADPDFFVSNCHKWLLVPRGCAMLYTPARTQCLLRTALATSHGYVPPSAAPAPPGSKSRYVANFEFVGTRDNGPYLCVADAIAWRERVCGGEENILRYLWALNKKGIRIVARALGTTHLDNETETLTNCAMGNVALPMRVDDEDASTALDAAPSAAIAAPDVVVARENVALVDKWMRERLFDDYKTFMTLFVMQDRYWVRLSAQIYLDEQDYEAAGDILKALCERIRRREYLVPQPVE

SEQ ID NO: 13 - DNA, 1113 bp, Mycolicibacterium smegmatis
atgctcgcgcagcagtggcgtgacgcccgtcccaaggttgccgggttgcacctggacagcggggcatgttcgcggcagagcttcgcggtgatcgacgcgaccaccgcacacgcacgccacgaggccgaggtgggtggttatgtggcggccgaggctgcgacgccggcgctcgacgccgggcgggccgcggtcgcgtcgctcatcggttttgcggcgtcggacgtggtgtacaccagcggatccaaccacgccatcgacctgttgctgtcgagctggccggggaagcgcacgctggcctgcctgcccggcgagtacgggccgaatctgtctgccatggcggccaacggtttccaggtgcgtgcgctaccggtcgacgacgacgggcgggtgctggtcgacgaggcgtcgcacgaactgtcggcccatcccgtcgcgctcgtacacctcaccgcattggcaagccatcgcgggatcgcgcaacccgcggcagaactcgtcgaggcctgccacaatgcggggatccccgtggtgatcgacgccgcgcaggcgctggggcatctggactgcaatgtcggggccgacgcggtgtactcatcgtcgcgcaagtggctcgccggcccgcgtggtgtcggggtgctcgcggtgcggcccgaactcgccgagcgtctgcaaccgcggatccccccgtccgactggccaattccgatgagcgtcttggagaagctcgaactaggtgagcacaacgcggcggcgcgtgtgggattctccgtcgcggttggtgagcatctcgcagcagggcccacggcggtgcgcgaacgactcgccgaggtggggcgtctctctcggcaggtgctggcagaggtcgacgggtggcgcgtcgtcgaacccgtcgaccaacccaccgcgatcaccacccttgagtccaccgatggtgccgatcccgcgtcggtgcgctcgtggctgatcgcggagcgtggcatcgtgaccaccgcgtgtgaactcgcgcgggcaccgttcgagatgcgcacgccggtgctgcgaatctcgccgcacgtcgacgtgacggtcgacgaactggagcagttcgccgcagcgttgcgtgaggcgccctga

SEQ ID NO: 14 - protein, 371 aa, Mycolicibacterium smegmatis
MMLAQQWRDARPKVAGLHLDSGACSRQSFAVIDATTAHARHEAEVGGYVAAEAATPALDAGRAAVASLIGFAASDVVYTSGSNHAIDLLLSSWPGKRTLACLPGEYGPNLSAMAANGFQVRALPVDDDGRVLVDEASHELSAHPVALVHLTALASHRGIAQPAAELVEACHNAGIPVVIDAAQALGHLDCNVGADAVYSSSRKWLAGPRGVGVLAVRPELAERLQPRIPPSDWPIPMSVLEKLELGEHNAAARVGFSVAVGEHLAAGPTAVRERLAEVGRLSRQVLAEVDGWRVVEPVDQPTAITTLESTDGADPASVRSWLIAERGIVTTACELARAPFEMRTPVLRISPHVDVTVDELEQFAAALREAP

SEQ ID NO: 15 - DNA, 2631 bp, Artificial sequence
  DNA sequences codon-optimised for Saccharomyces cerevisiae
  misc_feature (1)..(2631): NcEgt1 DNA sequence codon-optimised for S. cerevisiae
atgccatctgctgaatctatgactccatcttctgctttgggtcaattgaaagctactggtcaacatgtcttgtccaagttgcaacaacaaacttccaacgccgatatcatcgatattagaagagttgccgttgagatcaacttgaaaaccgaaattacctccatgttcagaccaaaagatggtccaagacaattgccaaccttgttgttgtataacgaaagaggcttgcagttgttcgaaagaattacttacttggaagagtactacttgaccaacgacgagattaagattttgactaagcacgctactgaaatggcctcttttattccatctggtgccatgattatcgaactaggttctggtaatttgaggaaggtcaacttgttgttagaagctttggataatgctggtaaggccattgattattacgccttggatttgtccagagaagaattggaaagaaccttggctcaagtcccatcttacaaacatgttaagtgtcatggtttgttgggtacttacgatgatggtagagattggttgaaagctccagaaaacatcaacaagcaaaagtgcatactgcatctgggttcttctattggtaacttcaatagatctgatgctgccacttttttgaagggtttcactgatgttttgggtccaaacgataagatgttgattggtgttgatgcttgtaacgatccagctagagtttaccatgcttacaatgataaggttggtatcacccacgaattcatcttgaatggtttgagaaacgccaacgaaattattggtgaaaccgctttcattgaaggtgattggagagttatcggtgaatacgtttatgatgaagaaggtggtagacatcaagctttttatgctccaactagagataccatggttatgggtgaattgatcagatcccatgacagaatccaaatcgaacagtctctgaagtactccaaagaggaatctgaaagattgtggtctactgctggtttggaacaagtttctgaatggacttacggtaatgaatacggtttacatttgttggccaagtccagaatgtccttctcattgattccatcagtttacgctagatctgctttgccaactttggatgattgggaagctttgtgggctacttgggatgttgttactagacaaatgttgccacaagaggaattattggagaagccaatcaagttgagaaatgcctgcattttctacttgggtcatatcccaactttcttggatattcagttgactaagactaccaagcaagctccatctgaaccagctcatttctgtaagattttcgaaaggggtatcgatccagatgttgacaatccagaattgtgtcatgcccattctgaaattccagatgaatggccaccagttgaagaaattttgacttaccaagaaaccgtcagatctagattgagaggtctatatgctcatggtattgccaacattccaagaaatgtcggtagagctatttgggttggtttcgaacatgaattgatgcacatcgagactctgttgtacatgatgttgcaatctgacaagaccttgattccaactcatattccaagaccagatttcgataagttggctagaaaagccgaatcagaaagggttccaaatcaatggtttaagatcccagctcaagaaatcactattggtttggatgaccctgaagatggttccgatattaacaaacattacggttgggataacgagaagccaccaagaagagttcaagttgctgcttttcaagctcaaggtagaccaattacaaacgaagaatacgcccaatacttgttggaaaagaacattgataagttgccagcttcttgggctagattggataacgaaaacatttctaacggcaccaccaattctgtttctggtcatcattctaacagaacctccaaacaacaactgccatcttcattcttggaaaaaactgctgttagaaccgtttacggtttggttccattgaaacatgctttggattggccagtttttgcttcctatgatgaattggctggttgtgctgcttatatgggtggtagaattccaactttcgaagaaaccagatctatctacgcttatgctgatgctctgaagaagaagaaagaagctgaaagacaactgggtagaactgttccagctgttaatgctcatttgactaacaacggtgttgaaattactcctccatcatcaccatcatctgaaactccagcagaatcttcttcaccatctgattctaacactaccttgattaccaccgaggatttgttctctgatttggatggtgctaatgttggtttccataattggcatccaatgcctattacttctaagggtaataccttggtcggtcaaggtgaattaggtggtgtttgggaatggacatcttccgttttgagaaaatgggaaggttttgagccaatggaattatacccaggttacactgctgatttctttgacgaaaagcacaacatcgttttaggtggttcatgggctactcatccaagaattgctggtagaaagtcttttgtcaactggtatcaaagaaactacccatatgcatgggttggtgctagagttgttagagatttgtga

SEQ ID NO: 16 - DNA, 2529 bp, Artificial sequence
  DNA sequence codon-optimised for Saccharomyces cerevisiae
  misc_feature (1)..(2529): CpEgt1 DNA sequence codon-optimised for S. cerevisiae
atgactgccgttaagcaaattcctgaaagaaaggtgttgatagattcaaatcataagtctccatcaaaaccgggtaaacatcctaattctgtcattgatatcaggtctaataaggacgatttaaatttacgtcatgccctagtctcatcttttaatccacacgatggaaaacctaggtggctacctactatgttattgtacgacgaaaaaggtttacaattgtttgaagatataacttacttagatgagtattatttgactggctacgaaattgaattattgaagaaacattcagcagaaattgcagctgctattcctgatggttctatggtcatcgaattgggctctggtaatttgagaaagatctgtttgttgttacaagcctttgaggattcacataagtctatcgactactatgcattagatttatcacaaaaggaattagaaagaactttgagccatgttcctgactttaaatatgtctcttgtcatggactgctaggtacatatgatgatggtgttacatggttgaaacaaccaggtatagtcaataagactaagtgcatcatccatcttggttcgtctattgggaattttcatagaaatgaagctgccgatttcctgcagacatttgctgatgtaatgaaaccagacgactctatggttattggtcttgattcatgcggtaatccagagatgtctcgcattcaaagattcattttgaacggcttatccaatgctaatagcgtttatggcaaggaaatattctatgttccagattggagagtaattggtgaatatgtttacgatgatgaaggtggcagacaccaggcttttatttcacctttgaaagaagtcactgctttagggtctgttattaaagcccatgaaagaattaaaattgaacaatctttgaagtactctaaggcctcagctgacgatttatggagaaatgctggctttcgagaaactcaaacttggacgagaaacggtgaatatggactacatatgttgcaaagagctgatccgcccttctctaaggctccttctttgtatgcagctaatactcttccctctctttctgattggagagcattgtggtgtgcctgggatattgtcactagagctatgttgccacaacaggaattgactgagaaacctatagagttaagacatgcctacatcttttaccttggtcatattcctaccttcttagacatccagttaaccaaaacatcagcatgggctccaacctctccagtttcttatcatgccattttcgagcgcggcattgatcccgatgttgataacccagaaaagtgtcatgatcactcagagattccagatgaatggccaccagtcgaagaaattattgcttatcaagatagggtgcgtgttagattgacagaactgtataaacagggtgtgcacacaattacaagaaaggctgctagagctatctgggtttcatttgaacatgaagctatgcatttggaaaccttgttgtatatgatgctacaaagtgataaagtgttgccacctccacacactggcgttccagactttgaaagaatggcaactaaggctttcgaagctcgtacgcaaaatatgtggttcgaaattccagaacagactattagtcttggaacagatgatccagaagatggggatgaagacgttcattttggatgggacaacgaaaaaccagttagaagagttaaggttcacgcgttgcaagctcaaggaagaccaattacaaatgaggaatacgcattatatatttaccataccaactcttctaaactgccagcatcttggagttcgtccccttcatcttctctgtctaacggcgtgtctcatcccagctcccataacaagcatattccaactgatttgcctcattccttcttgcaaggtaagtttgttagaaccgtatatggtttgatacctttatctttggcgttggattggcctgttcaagcttcttatgatgaattagctgactgtgcattatggatgggtggaagaattccaaccttagaggaagccagatcaatctatgcctttgttgaatctaaaacgcaaatagcaacaggtaacacattggtcaagaaagttcctgctgttaatggacacttggttaataacggagttgaggaaactccaccacatgaatcctcttcggcagttgagaattctttattcatcgacttagccggtttgaacgtgggttttaaaagttggaatcctgaacctgttacatcttctggtacgtctttggctggacaatcctctatgggtggtgtatgggagtggacctcttctgttttaagaccacatgaagggttccacccaatggagttgtatcctggttatacagccgatttctttgatgaaaaacataatattgttctcggaggatcatgggctactcatccaagaatagcgggtagaaaaagctttgttaactggtatcaaagaaactatccgtacgcctgggctggtgccagacttgttaaagatgcttga

SEQ ID NO: 17 - DNA, 1422 bp, Artificial sequence
  DNA sequence codon-optimised for Saccharomyces cerevisiae
  misc_feature (1)..(1422): NcEgt2 DNA sequence codon-optimised for S. cerevisiae
atggttgctactactgttgaattgccattgcaacaaaaagctgatgctgctcaaactgttactggtccattgccatttggtaacagcttgttgaaagaattcgttttggatccagcctacagaaacttgaatcatggttcttttggtactatcccatccgctattcaacagaagttgagatcttatcaaactgctgctgaagctagaccatgtccatttttgagatatcaaaccccagttttgttggacgaatctagagctgctgttgctaatttgttgaaggttccagttgaaaccgttgttttcgttgctaatgctactatgggtgtcaacactgttttgagaaatatcgtttggtctgctgatggtaaggacgaaatcttgtactttgatacaatctacggtgcttgcggtaagaccattgattatgttatcgaagataagaggggcatcgtttcctctagatgtattccattgatatacccagccgaagatgatgatgttgttgcagcttttagagatgccatcaagaagtctagagaagaaggtaaaagaccaagattggccgttatcgatgttgtttcttctatgccaggtgttagattcccattcgaagatatcgttaagatctgcaaagaggaagagatcatttcttgcgttgatggtgctcaaggtattggtatggttgatttgaagattaccgaaaccgatccagacttcctgatttctaattgtcataagtggttgttcaccccaagaggttgtgctgttttttatgttccagtcagaaaccagcacttgatcagatctactttgccaacttctcatggtttcgttccacaagttggtaatagattcaatccattggttccagctggtaacaagtctgcttttgtttctaacttcgaattcgttggtactgtcgataactctccattcttctgtgttaaggatgctattaagtggcgtgaagaggttttaggtggtgaagaaagaattatggagtacatgactaagttggctagagaaggtggtcaaaaggttgctgaaattttgggtactagagtcttggaaaactctaccggtacattgattagatgcgccatggttaatattgccttgccttttgttgttggtgaagatccaaaagctccagttaagttgaccgaaaaagaagaaaaagacgtcgaaggcttgtacgaaattccacatgaagaggctaatatggctttcaagtggatgtacaacgtattgcaagatgagttcaataccttcgttccaatgacctttcatagacgtagattttgggctagattgtccgctcaagtttacttggaaatgtctgattttgaatgggctggcaagaccttaaaagaattgtgtgaaagggttgctaagggcgagtacaaagaatctgcttaa

SEQ ID NO: 18 - DNA, 1581 bp, Artificial sequence; identical to SEQ ID NO: 11
  DNA sequence codon-optimised for Saccharomyces cerevisiae
  misc_feature (1)..(1581): CpEgt2 DNA sequence codon-optimised for S. cerevisiae

SEQ ID NO: 19 - DNA, 1116 bp, Artificial sequence
  DNA sequence codon-optimised for Saccharomyces cerevisiae
  misc_feature (1)..(1116): MsEgtE DNA sequence codon-optimised for S. cerevisiae
atgatgttggctcaacaatggagagatgctagaccaaaagtcgccggtttgcacttagattctggtgcttgctctagacaatcttttgccgttattgacgcaactactgctcatgctaggcatgaagcagaagttggtggttatgttgcagctgaagccgctactccagctttagatgctggtagggctgctgtcgcctctttgattggttttgctgcatcagatgttgtttacacttctggttctaatcacgctattgatttactattgtcttcttggccaggtaaaagaactttagcctgtttgcccggtgaatatggtccaaatttgtctgctatggctgcaaatggttttcaagttagagctctgccagtggatgatgatggtagagttttggttgatgaagcttctcatgaattgtctgctcatccagttgccttagtccatttgaccgctttggcttctcatagaggtattgcccagccagcagctgaattggttgaagcttgtcataacgccggtatcccagttgttattgatgctgcacaggcattgggccatttagattgtaatgttggtgctgatgcggtctattcctcctctagaaaatggttggctggtccaaggggtgttggtgtactagctgttagaccagaattagctgaaagattacaaccaagaattccaccatctgattggccaatcccaatgtctgttttggaaaaattggaattaggtgagcataacgctgctgctagagttggtttttctgttgctgtgggtgaacatctcgcagctggaccaactgctgtcagggaaagattagctgaagttggtagattatctaggcaagtcttggctgaagttgatggatggagagtcgtcgaaccagttgatcaaccaactgcaattactactttagaatctaccgatggtgcagatccagcttctgttagatcttggttaatcgctgaaagaggtattgttactactgcttgtgagttggctagagctccatttgaaatgagaactccagtcctgagaatttctccacatgttgacgttacagttgatgaattagaacaatttgctgcagctttgagagaagctccatga

SEQ ID NO: 20 - protein, 392 aa, Schizosaccharomyces pombe
MAENNVYGHEMKKHFMLDPDYVNVNNGSCGTESLAVYNKHVQLLKEAQSKPDFMCNAYMPMYMEATRNEVAKLIGADSSNIVFCNSATDGISTVLLTFPWEQNDEILMLNVAYPTCTYAADFAKNQHNLRLDVIDVGVEIDEDLFLKEVEQRFLQSKPRAFICDILSSMPVILFPWEKVVKLCKKYNIVSIIDGAHAIGHIPMNLANVDPDFLFTNAHKWLNSPAACTVLYVSAKNHNLIEALPLSYGYGLREKESIAADTLTNRFVNSFKQDLPKFIAVGEAIKFRKSIGGEEKIQQYCHEIALKGAEIISKELGTSFIKPPYPVAMVNVEVPLRNIPSIETQKVFWPKYNTFLRFMEFKGKFYTRLSGAVYLEQSDFYYIAKVIKDFCSL

SEQ ID NO: 21 - protein, 392 aa, Schizosaccharomyces pombe; identical to SEQ ID NO: 20

SEQ ID NO: 22 - DNA, 1272 bp, Mycolicibacterium smegmatis
atggccttacccgccagaagtgattctggctgtgcggttccggtcgagttcaccagcgcggagcaggccgccgcccacatcggtgccaacagcctgcaggacggtccgatcggccgggtgggcctggagatcgaggcgcactgtttcgacctgagcaatccgacgcggcgaccgagctgggacgaactgtccgccgtgatcgcggacgtaccgccactgcccggtggcagccggatcaccgtggaacccggcggcgcggtcgaactgtccggtccgccgtacgacggtccgctcgccgcggtcgccgcgctgcaggccgaccgtgctgtgctgcgtgcggagttcgcccgcaggaatcttggcctggtgctgctcggcaccgatccgctgcggccgacccgccgggtcaacccgggtgcgcgctactcggccatggaacagttcttcaccgcgagcggtaccgccgaggccggcgccgcgatgatgacggcaaccgcgtcggtgcaggtcaacctggatgccggtccgcgcgacggctgggccgagcgggtccggttggcacatgcgctggggcccacgatgatcgcgatcaccgccaactcaccgatgctgggtggacagttcacgggctggtgttcgacgcggcaacgcgtgtgggggcagctcgactcggcgcggtgcgggccggtgctgggtgtggacggcgacgatcccgcctccgaatgggcgcgctacgcgctgcgcgcgccggtgatgctggtgaactctccggacgcggtgcccgtgaccaactgggtgccgttcgcggactgggccgacgggcgcgccgtgctgggtggacgcagaccgaccgaggccgacctggactatcacctgacgacgctgttcccgccggtgcggccgcgccgctggctggagatccgctatctcgacagcgtgcccgacgcgctgtggccggccgctgtgttcaccctcaccacattgctcgacgatcccgtcgcggccgaaagtgccgctgaggcaacacgtccagtcgccacagcctgggaccgcgccgcgcggatgggcctgacggaccgacatctgcacaccgcggccctgacgtgcgtgcgcctcgcggcggagcgggcaccggccgagctcgaggaatcgatgacgctgttgatgcgttcggtccagcagaggcgtagtcccgccgacgacttctcggaccgggtggtggcacgtggaatcgcagccgccgtccgggaactggcgaaaggtgagctttga

SEQ ID NO: 23 - DNA, 1272 bp, Mycolicibacterium smegmatis
atggctttgccagctagatctgattctggctgtgccgttccagtcgagttcacttctgctgaacaagctgctgcccatattggtgctaactctttacaagatggtccaattggtcgtgttggtctggaaattgaagctcactgtttcgatctgtctaatccaactcgtagaccatcttgggatgaattgtctgctgtcattgctgatgttcctccattgccaggaggttctagaataacagtggaacccggaggtgcagttgaattgtctggtccaccatatgatggtccattggctgctgttgctgctttacaagctgacagggccgtcttgagggctgaatttgctagaagaaatttaggcttggtcttgttaggtacagatccattgagaccaacgagaagagtgaacccaggtgctagatattctgctatggagcagttcttcactgcatcaggtactgctgaggctggtgccgctatgatgactgctactgcatctgtccaagttaatttggatgctggtccaagagatggttgggccgagagagttagattggctcatgctttaggtcccaccatgatcgccattactgctaattctccaatgctaggtggtcaatttaccggttggtgttctacaagacaaagagtttgggggcaattggattctgctagatgtggtcccgttttaggtgttgatggcgacgatccagcctcagaatgggccagatatgctttgagagctccagtgatgttagtgaattctccagatgctgtaccagttactaactgggtcccattcgctgattgggctgatgggagagctgtcttgggtggtagaagaccaactgaagctgacttggattatcatttaactactttatttcctccagttaggccacggagatggttagaaattagatatttagactcggttcccgacgctttatggccagctgcagttttcactttaactactttgttggatgatccagttgcagcagaatctgctgcggaagctactagaccagtagctactgcttgggatcgtgctgctagaatgggtttaactgatagacatttacacaccgcggctttaacttgtgtaagattagctgctgaaagagctccggctgaattggaagaatctatgacattattaatgagatctgttcaacaaagacggtcaccagctgatgatttttccgatagagttgttgctaggggtatcgctgccgcagttagagaattggcaaaaggtgaattgtga

SEQ ID NO: 24 - protein, 425 aa, Mycolicibacterium smegmatis
MVMALPARSDSGCAVPVEFTSAEQAAAHIGANSLQDGPIGRVGLEIEAHCFDLSNPTRRPSWDELSAVIADVPPLPGGSRITVEPGGAVELSGPPYDGPLAAVAALQADRAVLRAEFARRNLGLVLLGTDPLRPTRRVNPGARYSAMEQFFTASGTAEAGAAMMTATASVQVNLDAGPRDGWAERVRLAHALGPTMIAITANSPMLGGQFTGWCSTRQRVWGQLDSARCGPVLGVDGDDPASEWARYALRAPVMLVNSPDAVPVTNWVPFADWADGRAVLGGRRPTEADLDYHLTTLFPPVRPRRWLEIRYLDSVPDALWPAAVFTLTTLLDDPVAAESAAEATRPVATAWDRAARMGLTDRHLHTAALTCVRLAAERAPAELEESMTLLMRSVQQRRSPADDFSDRVVARGIAAAVRELAKGEL

SEQ ID NO: 25 - DNA, 1179 bp, Mycolicibacterium smegmatis
atgagcccgctcgtgtgggacctcgcgcacatcgggcagcaggaagaactgtggctgctgcgcgacggcaaccccgaccgccccggcatgctcgcacccgaggtggaccggctttacgacgcgttcgagcactcacgcgccagccgggtcaacctcccgttgctgccgccttcggatgcgcgcgcctactgcgcgacggtgcgggccaaggcgctcgacaccctcgacacgctgcccgaggacgatccgggcttccggttcgcgctggtgatcagccacgagaaccagcacgacgagaccatgctgcaggcactcaacctgcgcgagggcccacccctgctcgacaccggaattcccctgcccgcgggcaggccaggcgtggcaggcacgtcggtgctggtgccgggcggcccgttcgtgctcggggtcgacgcgctgaccgaaccgcactcactggacaacgaacggcccgcccacgtcgtggacatcccgtcgttccggatcggccgcgtgccggtcaccaacgccgaatggcgcgagttcatcgacgacggtggctacgaccaaccgcgctggtggtcgccacgcggctgggcgcaccgccaggaggcgggcctggtggccccgcagttctggaaccccgacggcacccgcacccggttcgggcacatcgaggagatcccgggtgacgaacccgtgcagcacgtgacgttcttcgaagccgaggcctacgcggcgtgggccggtgctcggttgcccaccgagatcgaatgggagaaggcctgcgcgtgggatccggtcgccggtgctcggcgccggttcccctggggctcagcacaacccagcgcggcgctggccaacctcggcggtgacgcacgccgcccggcgccggtcggggcctacccggcgggggcgtcggcctatggcgccgagcagatgctgggcgacgtgtgggagtggacctcctcgccgctgcggccgtggcccggtttcacgccgatgatctacgagcgctacagcacgccgttcttcgagggcaccacatccggtgactaccgcgtgctgcgcggcgggtcatgggccgttgcaccgggaatcctgcggcccagcttccgcaactgggaccacccgatccggcggcagatcttctcgggtgtccgcctggcctgggacgtctga

SEQ ID NO: 26 - DNA, 1287 bp, Mycolicibacterium smegmatis
atgattgccagagaaactttggctgatgaattggctttggctagagaaagaactttgagattggttgaattcgatgatgccgaattgcatagacagtacaatccattgatgtctccattggtttgggacttagctcatattggtcaacaagaggaattgtggttgttgagagatggtaatccagatagaccaggtatgttggctcctgaagttgatagattatacgatgccttcgaacattccagagcttctagagttaatttgccattattgccaccatctgatgctagagcttattgtgctactgttagagctaaagctttggataccttggatactttgccagaagatgatccaggttttagattcgccttggttatctctcacgaaaatcaacatgacgaaaccatgttgcaagccttgaatttgagagaaggtccaccattattggatactggtattccattgccagctggtagacctggtgttgctggtacttctgttttggttccaggtggtccatttgttttgggtgttgatgctttgactgaaccacattctttggataacgaaagaccagctcatgttgttgatatcccatctttcagaattggtagagttccagttactaatgctgaatggcgtgaattcattgatgatggtggttatgatcaacctagatggtggtcacctagaggttgggctcatagacaagaagctggtttggttgctccacaattttggaatccagatggtactagaactagattcggtcacattgaagaaatcccaggtgatgaaccagttcaacatgttacttttttcgaagctgaagcttatgctgcttgggctggtgctagattgccaactgaaattgaatgggaaaaagcttgtgcttgggatccagttgctggtgctcgtagaagatttccatggggttctgctcaaccatctgctgctttagctaacttaggtggtgatgcaagaaggccagctccagttggtgcttatccagctggtgcatctgcttatggtgctgaacaaatgttgggtgatgtttgggaatggacatcttctccattaagaccatggcctggttttactccaatgatctacgaaagatactctaccccattcttcgaaggtactacttctggtgattacagagttttgagaggtggttcatgggctgttgctccaggtattttaagaccttcttttagaaactgggatcacccaattagaaggcaaatcttttcaggtgttagattggcttgggatgtctga

SEQ ID NO: 27 - protein, 428 aa, Mycolicibacterium smegmatis
MIARETLADELALARERTLRLVEFDDAELHRQYNPLMSPLVWDLAHIGQQEELWLLRDGNPDRPGMLAPEVDRLYDAFEHSRASRVNLPLLPPSDARAYCATVRAKALDTLDTLPEDDPGFRFALVISHENQHDETMLQALNLREGPPLLDTGIPLPAGRPGVAGTSVLVPGGPFVLGVDALTEPHSLDNERPAHVVDIPSFRIGRVPVTNAEWREFIDDGGYDQPRWWSPRGWAHRQEAGLVAPQFWNPDGTRTRFGHIEEIPGDEPVQHVTFFEAEAYAAWAGARLPTEIEWEKACAWDPVAGARRRFPWGSAQPSAALANLGGDARRPAPVGAYPAGASAYGAEQMLGDVWEWTSSPLRPWPGFTPMIYERYSTPFFEGTTSGDYRVLRGGSWAVAPGILRPSFRNWDHPIRRQIFSGVRLAWDV

SEQ ID NO: 28 - DNA, 684 bp, Mycolicibacterium smegmatis
atgtgccggcatgtggcgtggctgggcgcgccgcggtcgttggccgacctggtgctcgacccgccgcagggactgctggtgcagtcctacgcaccgcgacgacagaagcacggtctgatgaacgccgacggttggggcgcagggtttttcgacgacgagggagtggcccgccgctggcgcagcgacaaaccgctgtggggtgatgcgtcgttcgcgtcggtggcacccgcactacgcagtcgttgcgtgctggccgcggtgcgctcggccaccatcggcatgcccatcgaaccgtcggcgtcggcgccgttcagcgacgggcagtggctgctgtcgcacaacggcctggtcgaccgcggggtgctcccgttgaccggtgccgccgagtccacggtggacagcgcgatcgtcgcggcgctcatcttctcccgtggcctcgacgcgctcggcgccaccatcgccgaggtcggcgaactcgacccgaacgcgcggttgaacatcctggccgccaacggttcccggctgctcgccaccacctggggggacacgctgtcggtcctgcaccgccccgacggcgtcgtcctcgcgagcgaaccctacgacgacgatcccggctggtcggacatcccggaccggcacctcgtcgacgtccgcgacgcccacgtcgtcgtgacacccctgtga

SEQ ID NO: 29 - DNA, 684 bp, Mycolicibacterium smegmatis
atgtgcagacatgttgcttggttgggtgctccaagatctttggctgatttggttttggatccaccacaaggtttgttggttcaatcttatgctcctagaaggcaaaaacacggtttgatgaatgctgatggttggggtgctggtttttttgatgatgaaggtgttgctagaagatggcgttctgataagcctttgtggggtgatgcttcttttgcttctgttgctccagctttgagatctagatgtgttttggctgctgttagatctgctactattggtatgccaattgaaccatctgcttcagctccattttctgatggtcaatggttgttgtctcataacggtttggttgatagaggtgttttgccattgactggtgctgctgaatctactgttgattctgctatagttgctgccttgattttctctagaggtttggatgctttgggtgctacaattgctgaagttggtgaattagatccaaacgccagattgaatattttggccgctaatggttctaggttgttggctactacttggggtgatactttgtctgttttacacagaccagatggtgttgttttagcttctgaaccatatgatgatgatccaggttggtctgatattccagatagacacttggttgatgttagagatgctcatgttgttgttaccccattgtga

SEQ ID NO: 30 - protein, 227 aa, Mycolicibacterium smegmatis
MCRHVAWLGAPRSLADLVLDPPQGLLVQSYAPRRQKHGLMNADGWGAGFFDDEGVARRWRSDKPLWGDASFASVAPALRSRCVLAAVRSATIGMPIEPSASAPFSDGQWLLSHNGLVDRGVLPLTGAAESTVDSAIVAALIFSRGLDALGATIAEVGELDPNARLNILAANGSRLLATTWGDTLSVLHRPDGVVLASEPYDDDPGWSDIPDRHLVDVRDAHVVVTPL

SEQ ID NO: 31 - DNA, 966 bp, Mycolicibacterium smegmatis
atgacgctctcactggccaactacctggcagccgactcggccgccgaagcactgcgccgtgacgtccgcgcgggcctcaccgcggcaccgaagagtctgccgcccaagtggttctacgacgccgtcggcagtgatctgttcgaccagatcacccggctccccgagtattaccccacccgcaccgaggcgcagatcctgcggacccggtcggcggagatcatcgcggccgcgggtgccgacaccctggtggaactgggcagtggtacgtcggagaaaacccgcatgctgctcgacgccatgcgcgacgccgagttgctgcgccgcttcatcccgttcgacgtcgacgcgggcgtgctgcgctcggccggggcggcaatcggcgcggagtaccccggtatcgagatcgacgcggtatgtggcgatttcgaggaacatctgggcaagatcccgcatgtcggacggcggctcgtggtgttcctggggtcgaccatcggcaacctgacacccgcgccccgcgcggagttcctcagtactctcgcggacacgctgcagccgggcgacagcctgctgctgggcaccgatctggtgaaggacaccggccggttggtgcgcgcgtacgacgacgcggccggcgtcaccgcggcgttcaaccgcaacgtgctggccgtggtgaaccgcgaactgtccgccgatttcgacctcgacgcgttcgagcatgtcgcgaagtggaactccgacgaggaacgcatcgagatgtggttgcgtgcccgcaccgcacagcatgtccgcgtcgcggcactggacctggaggtcgacttcgccgcgggtgaggagatgctcaccgaggtgtcctgcaagttccgtcccgagaacgtcgtcgccgagctggcggaagccggtctgcggcagacgcattggtggaccgatccggccggggatttcgggttgtcgctggcggtgcggtga

SEQ ID NO: 32 - DNA, 966 bp, Mycolicibacterium smegmatis
atgactttgtccttggctaattacttggctgctgattctgctgctgaagctttgagaagagatgttagagctggtttgactgctgctccaaaatctttgccaccaaaatggttttatgatgccgttggttctgatttgttcgatcagattactagattgccagagtactacccaactagaactgaagctcaaattttgagaaccagatccgctgaaattattgctgctgctggtgctgatactttggttgaattaggttctggtacttccgaaaagaccagaatgttgttggatgctatgagagatgccgaattgctgagaagattcattccatttgatgttgatgccggtgttttgagatcagctggtgcagctattggtgctgaatatccaggtattgaaattgatgctgtttgcggtgatttcgaagaacatttgggtaagattccacacgttggtagaagattggttgttttcttgggttctaccattggtaatttgactccagctccaagagctgaatttttgtctactttggctgataccttgcaaccaggtgattctttgttgttgggtactgatttggttaaggataccggtagattggttagagcttatgatgatgcagctggtgttacagctgcttttaatagaaatgttttggccgtcgtcaacagagaattgtctgctgattttgatttggatgccttcgaacatgttgctaagtggaattctgatgaagaaaggatcgaaatgtggttgagagctagaactgctcaacatgttagagttgctgcattggatttggaagttgattttgccgctggtgaagaaatgttgactgaagtttcttgtaagttcaggccagaaaacgttgttgctgaattggctgaagctggtttaagacaaactcattggtggactgatcctgctggtgattttggtttgtctttggctgttagataa

SEQ ID NO: 33 - protein, 321 aa, Mycolicibacterium smegmatis
MTLSLANYLAADSAAEALRRDVRAGLTAAPKSLPPKWFYDAVGSDLFDQITRLPEYYPTRTEAQILRTRSAEIIAAAGADTLVELGSGTSEKTRMLLDAMRDAELLRRFIPFDVDAGVLRSAGAAIGAEYPGIEIDAVCGDFEEHLGKIPHVGRRLVVFLGSTIGNLTPAPRAEFLSTLADTLQPGDSLLLGTDLVKDTGRLVRAYDDAAGVTAAFNRNVLAVVNRELSADFDLDAFEHVAKWNSDEERIEMWLRARTAQHVRVAALDLEVDFAAGEEMLTEVSCKFRPENVVAELAEAGLRQTHWWTDPAGDFGLSLAVR

SEQ ID NO: 34 - DNA, 1116 bp, Mycolicibacterium smegmatis; identical to SEQ ID NO: 19

SEQ ID NO: 35 - DNA, 1206 bp, Mycolicibacterium smegmatis
atgccattttccctgtacccacttgcagttgctgtgttcgctatgggaacctctgaatttatgttggcaggattggttcctgatattgcagctgacctcggagtgtctattggctctgctggactgttgacttctgcttttgctgttggaatggtcgtgggagctccttctatggcagctctgacaagaagatggcgcgctagagtgtctctgagcgcttttctccttaccttcgctctcgtgcatgttctgggagccgttaccacctcttttggagtgctgctggtgacaagacttgtggcagctgtggctaatgctggattcctggccgttgccctgagtacagcagcaacactggtgccagctggaaggcagggacgtggacttgcagttctgcttgccggaacaaccctcgcaacaattgctggagtgccaggaggagctgtgcttggaacaatgttgggatggagagcaaccttttgggcaattgctctgctgtgcctgccagcagttgttggcattgcaaccgcacttcctgctggcgctggtagagccggttggcccgttgccggagccagtctgtgcgatgaactggcccagctgggaagaaaaagactggctctggctatgcttcttgctgcactggtcaatgctggaacatttgctacctttacatttcttgctccaattgtgacagaaagtgctggacttggccgcctgtgggtgtctgtggtgctgcttctgttcggattcggaagctttattggagtgacagtcgccggaaggctcagcgatacaagacctggaattgtgatcggcgcaggtggacctgctctgctggctggatgggccgctcttgcacttctgtcctctcagccagtggcactgttgccacttgcattcgtgcagggagcactgagctttgccgttggatccacattgattacaagggtgctgtatgaggcatcagctgccccaacaatgggtggagcttatgctactgcagcactgaatgtcggagcagccggtggaccagtcgcagctgcagccgctctgggtaatcatgcaaacgtggtggcaccagtttgggttagttccgtgatggtggcattggctcttctcatcgctgttccaatgctgaagattgtggctccaagaccaaatccagccacatctacaccaccactgggaggaaattgcggataa

SEQ ID NO: 36 - DNA, 1656 bp, Homo sapiens
atgcgtgactatgatgaggtcatcgcattccttggcgaatggggaccattccagcgcttgatcttctttctgcttagcgcctctattatccctaatggctttaatggtatgtccgttgtcttcctggccggtacccctgaacatcgctgtagagtgccagacgccgcaaacctgagcagcgcctggcgcaacaactctgtccctctgagactgcgtgatggccgcgaggtcccccacagctgtagccgctacagactggccaccatcgccaacttctccgctctcggactggagccaggtcgcgatgttgatctgggacagctggaacaggagagctgtctggatggctgggagttcagccaggctgtctacctgtccaccgtcgtgaccgaatggaatctggtttgtgaggacaactggaaggtgccactgaccacctccctgttcttcgttggcgtccttctgggctccttcgtgtccggtcagctgtcagatagatttggcaggaagaacgttctcttcgcaaccatggctgtacagactggcttcagcttcctgcagattttctccatcagctgggagatgttcactgttttgtttgtcatcgtgggcatgggccagatctccaactatgtcgttgccttcatccttggaacagaaattcttggcaagtcagttcgtattattttctctacattaggagtgtgcacattttttgcagttggctatatgctgctgccactgtttgcttacttcatcagagactggcgtatgctgctgctggccctgacggttcctggagtgctgtgtgtcccactgtggtggttcattcctgaatctcccagatggctgatctcccagagaagatttagagaggctgaagatatcatccaaaaagctgcaaaaatgaacaacatcgctgtcccagcagtcatttttgattctgttgaagagctgaatcctctgaagcagcagaaagctttcattctggatctgttcagaactagaaatattgccattatgaccattatgtctttgctgctttggatgctgacctcagtgggttactttgctctgtctctggatgctcctaatttgcatggagatgcctacctgaactgtttcctgtctgccttgattgaaattccagcttacattacagcctggctgctgttgagaaccctgccaaggcgttatatcatcgctgcagttctgttctggggaggaggtgttcttcttttcattcaactggtgcctgtcgattattacttcttgtccattggtctggtcatgctgggaaaatttggtatcacctctgctttctccatgctgtatgtcttcactgctgaactgtacccaaccctggtcaggaacatggctgtgggtgtcacatccacggcctccagagttggcagcatcattgccccctactttgtttacctcggtgcttacaacagaatgctgccttacatcgtcatgggttctctgactgtcctgattggaatccttaccctttttttccctgaatccttgggaatgactcttccagaaaccttagaacagatgcagaaagtgaaatggttcagatctggaaaaaaaacaagagactcaatggagacagaggaaaatcccaaggttcttattactgcattctaa

SEQ ID NO: 37 - protein, 553 aa, Arabidopsis thaliana
MAFSELLDLVGGLGRFQVLQTMALMVSIMWLCTQSMLENFSAAVPSHRCWAPLLDNSTAQASILGSLSPEALLAISIPPGPNQRPHQCRRFRQPQWQLLDPNATATSWSEADTEPCVDGWVYDRSIFTSTIVAKWNLVCDSHALKPMAQSIYLAGILVGAAACGPASDRFGRRLVLTWSYLQMAVMGTAAAFAPAFPVYCLFRFLLAFAVAGVMMNTGTLLMEWTAARARPLVMTLNSLGFSFGHGLTAAVAYGVRDWTLLQLVVSVPFFLCFLYSWWLAESARWLLTTGRLDWGLQELWRVAAINGKGAVQDTLTPEVLLSAMREELSMGQPPASLGTLLRMPGLRFRTCISTLCWFAFGFTFFGLALDLQALGSNIFLLQMFIGVVDIPAKMGALLLLSHLGRRPTLAASLLLAGLCILANTLVPHEMGALRSALAVLGLGGVGAAFTCITIYSSELFPTVLRMTAVGLGQMAARGGAILGPLVRLLGVHGPWLPLLVYGTVPVLSGLAALLLPETQSLPLPDTIQDVQNQAVKKATHGTLGNSVLKSTQF

SEQ ID NO: 38 - DNA, 1662 bp, Arabidopsis thaliana
atggctttttcagaattattggatttggttggtggtcttggtagatttcaagttttgcaaacaatggctttgatggtttctattatgtggctatgtactcaatctatgctggagaacttttctgctgctgttccatcacatagatgctgggctccattgttggataattctaccgctcaagcatcaattttaggttctttgtctccagaggctttattagcaatttctattccaccaggtcctaatcaaagaccacatcaatgtagaagatttagacaaccacaatggcaattattagatcctaatgctactgctacctcttggtcagaagctgatactgaaccatgtgtcgatggttgggtttatgatagatctatttttacttcgactatagtcgctaaatggaatttggtttgtgattcacatgccctaaagccaatggctcaatctatctacttagctggcattctagttggtgctgctgcgtgtggtcctgctagcgataggtttggtagaagattggtgttgacatggtcttatttgcaaatggctgttatgggtacagctgccgcctttgctccagcttttccagtttattgtcttttcaggtttttgttggcctttgctgttgctggtgttatgatgaatactggcacattgttaatggaatggaccgcagctagggctagacctttagttatgactttgaactccttaggttttagttttggtcatggtttaactgcggcagttgcttatggagttagagattggactttgttgcaattggttgtgtctgttccattctttttgtgctttttgtactcttggtggttagctgaatctgctaggtggttgttgaccacaggtagattagattggggtttacaggagttgtggagagttgctgctatcaatggtaagggtgctgttcaagacactttaactccagaagtcttattgtccgcaatgcgtgaagaattatctatgggtcaaccgcccgcctccttgggtactttattgagaatgccgggtttgagatttaggacatgtatttcaactttatgttggttcgcttttggttttactttctttggtttggctttggacttgcaagctttgggctctaacatttttctattgcaaatgtttattggtgtcgtggatattccagctaaaatgggagcattacttttgttgtcccacttgggtagaagacctacattggctgcttctttattgttagccggtttgtgtattttagccaataccttggttccacatgaaatgggtgctttgagatccgccctagctgttttgggattgggtggtgttggcgcggctttcacttgtattactatttattcatcagaattattccctaccgttttgagaatgactgctgtcggcttaggccaaatggcagctagaggaggtgctattttaggtccattagtgagattgttgggtgttcatggtccatggttgcctttattggtttacggtactgtgccagttctcagcggtttggcagctttgttgttaccagaaactcaaagtttaccattgccagatactatacaagatgttcagaatcaagctgttaagaaagctactcatggtacattgggtaatagtgttttaaaatcaactcaattttaa

SEQ ID NO: 39 - protein, 586 aa, Saccharomyces cerevisiae
MSRSNSIYTEDIEMYPTHNEQHLTREYTKPDGQTKSEKLNFEGAYINSHGTLSKTTTREIEGDLDSETSSHSSDDKVDPTQQITAETKAPYTLLSYGQKWGMVAILTMCGFWSSLGSPIYYPALRQLEKQFNVDENMVNVTVVVYLLFQGISPTVSGGLADCFGRRPIILAGMLIYVIASIGLACAPSYGVIIFLRCIQSIGISPTIAISSGVVGDFTLKHERGTFVGATSGFVLLGQCFGSLIGAVLTARWDWRAIFWFLTIGCGSCFLIAFLILPETKRTIAGNLSIKPKRFINRAPIFLLGPVRRRFKYDNPDYETLDPTIPKLDLSSAGKILVLPEIILSLFPSGLLFAMWTLMLSSISSGLSVAPYNYHLVIIGVCYLPGGIGGLMGSFFTGRIIDMYFKRKIKKFEQDKANGLIPQDAEINMFKVRLVCLLPQNFLAVVAYLLFGWSIDKGWRIESILITSFVCSYCAMSTLSTSTTLLVDLYPTKSSTASSCFNFVRCSLSTIFMGCFAKMKAAMTVGGTFTFLCALVFFFNFLMFIPMKYGMKWREDRLLKQQRQSWLNTLAVKAKKGTKRDQNDNHN

SEQ ID NO: 40 - DNA, 1761 bp, Saccharomyces cerevisiae
atgtcacgaagtaacagtatatacacagaagatattgagatgtatcctactcacaatgagcagcatctgacgagagaatacacaaaaccagatggtcaaacgaaaagtgaaaaactaaatttcgaaggcgcttatatcaacagtcacgggactctatccaagactaccacaagggaaatagaaggcgatttagactctgagacttcttctcactccagtgatgataaagttgatccaacccaacagataactgcagagacaaaagcgccatatactttactaagttacggtcagaagtggggaatggttgcaattctaacaatgtgtggcttttggtcttcattgggatcaccgatctattatcccgctttgaggcagttggaaaagcaatttaatgtggatgaaaatatggttaacgttactgtggttgtgtatctgttgtttcaaggtatatctcccacagttagcggtggtctggcggattgctttggacggagacctataattttagcaggtatgctgatatacgttattgcatccatcgggctagcatgtgctccatcatacggtgttattatcttcttgaggtgtattcagagtattggtatttcccccacaattgcaatcagttctggtgttgtcggggacttcactttgaagcatgaaagaggtacatttgttggcgccacctcagggtttgttttactgggtcaatgttttggtagtcttattggtgccgtcctgactgcaaggtgggattggagagctatcttttggtttttgaccattggttgtggaagttgttttctaattgcctttcttattttgccagaaacaaagcgaaccattgcaggtaacctttccattaagcctaaaagattcatcaacagagcgccgattttcctactgggcccagttagaaggagattcaagtacgataatcctgattatgagacattagatcccaccattccgaaactagatttgtcatccgccgggaagatccttgtattgccagaaattattctgtctttatttccatcagggctcttatttgctatgtggacgttaatgctttcctctatttcatccggtttatcagtggcgccatacaactatcatttggtcattattggtgtgtgttatctacccggtggtattggcggtttaatgggttccttcttcacaggtagaattattgacatgtatttcaagagaaagatcaagaaattcgagcaagacaaagctaacgggttgattccacaagacgcagagattaatatgttcaaagtccgtttagtttgtcttctgccccaaaatttcttggccgtcgtagcctatcttttatttggttggagtatagataagggctggaggatcgaatctattttaattacttcatttgtctgttcatactgtgctatgagtacattatcaacatcaactacgttattggtcgatttgtacccaacgaaatcatctacagcaagtagttgtttcaactttgttaggtgcagtttgagtaccatcttcatgggttgctttgccaaaatgaaagctgcgatgactgtaggtggtacttttacatttttatgtgcgctggtatttttcttcaacttcttaatgtttattccgatgaaatatggtatgaagtggagggaggatagattattgaaacaacaaagacagtcttggttaaacaccttggcagtcaaagccaaaaagggaacaaaaagagaccagaacgataatcataattaa

SEQ ID NO: 41 - protein, 577 aa, Homo sapiens
MGSRHFEGIYDHVGHFGRFQRVLYFICAFQNISCGIHYLASVFMGVTPHHVCRPPGNVSQVVFHNHSNWSLEDTGALLSSGQKDYVTVQLQNGEIWELSRCSRNKRENTSSLGYEYTGSKKEFPCVDGYIYDQNTWKSTAVTQWNLVCDRKWLAMLIQPLFMFGVLLGSVTFGYFSDRLGRRVVLWATSSSMFLFGIAAAFAVDYYTFMAARFFLAMVASGYLVVGFVYVMEFIGMKSRTWASVHLHSFFAVGTLLVALTGYLVRTWWLYQMILSTVTVPFILCCWVLPETPFWLLSEGRYEEAQKIVDIMAKWNRASSCKLSELLSLDLQGPVSNSPTEVQKHNLSYLFYNWSITKRTLTVWLIWFTGSLGFYSFSLNSVNLGGNEYLNLFLLGVVEIPAYTFVCIAMDKVGRRTVLAYSLFCSALACGVVMVIPQKHYILGVVTAMVGKFAIGAAFGLIYLYTAELYPTIVRSLAVGSGSMVCRLASILAPFSVDLSSIWIFIPQLFVGTMALLSGVLTLKLPETLGKRLATTWEEAAKLESENESKSSKLLLTTNNSGLEKTEAITPRDSGLGE

SEQ ID NO: 42 - DNA, 1734 bp, Homo sapiens
atgggatcaagacattttgaaggcatttatgatcatgtcggtcattttggtcgctttcaaagagttttgtatttcatctgtgctttccaaaatatttcttgcggtattcactacctggcttcagtttttatgggtgttactccacatcatgtttgtcgtccaccaggtaatgtttcacaagttgttttccacaatcattctaactggtccctggaagatactggtgctttattgtcatctggtcaaaaggattacgttacagttcaattgcaaaacggtgagatttgggagttaagtagatgttccagaaataagcgagagaatacttcttcactcggttatgaatatacaggttctaaaaaggaatttccatgcgttgatggttacatttacgatcaaaacacttggaaatctaccgccgtcacccaatggaatttggtttgtgatagaaaatggttggctatgttgattcaacccttatttatgtttggtgtcttattaggttctgtaacctttggttatttctccgatagattgggtaggagagtcgtgttgtgggctacttcatcatctatgtttttatttggtatcgcagctgcttttgctgttgattattatacttttatggccgctagattctttttagctatggttgcttccggatatttagtagttggttttgtttatgtgatggaatttattggtatgaaatctagaacttgggcttctgttcacttgcattctttctttgctgtcggtaccctgttggttgctttgactggttatttggttagaacttggtggctctaccaaatgattttaagtaccgttactgttccattcattttgtgttgttgggttttgccagaaacgccattttggttattatcagaaggtagatatgaggaagctcagaaaattgttgatattatggctaaatggaatagagcttcttcttgtaaattgtctgaattgttgtccttagatttgcaaggtccagttagtaatagtccaaccgaggttcaaaagcataatctatcatacttgttttataattggtccatcaccaagagaactttaactgtatggctgatttggttcaccggttctttgggattttattcttttagcctgaactctgttaatctgggtggtaacgaatacttaaatttatttttgttgggcgttgttgaaattccagcatacacatttgtgtgcattgccatggataaggttggtagaagaactgtcctggcttattctttgttttgttccgccttggcttgcggagtggtgatggttattccacaaaaacattatattttaggcgttgtcacggctatggttggtaaatttgcaattggtgccgcttttggtctaatctatttgtatactgctgaattatacccaacaatagtaagatctttagctgttggttctgggagcatggtttgtagattagcttctattctggctccattctctgttgatttgtcgtctatctggattttcattccacaattatttgttggtactatggctttgttgtccggtgttttgactcttaaattgcccgaaactctaggtaaaagattggctaccacttgggaggaagctgctaaattggaatctgaaaatgaatctaaatcaagcaaattattgctaactactaacaattctggtttagagaaaactgaagctattactccaagagattctggtttgggtgaataa

SEQ ID NO: 43 - protein, 491 aa, Homo sapiens
MGWGGGGGCTPRPPIHQQPPERRVVTVVFLGLLLDLLAFTLLLPLLPGLLESHGRAHDPLYGSWQGGVDWFATAIGMPVEKRYNSVLFGGLIGSAFSVLQFLCAPLTGATSDCLGRRPVMLLCLMGVATSYAVWATSRSFAAFLASRLIGGISKGNVSLSTAIVADLGSPLARSQGMAVIGVAFSLGFTLGPMLGASLPLEMAPWFALLFAASDLLFIFCFLPETLPLEKRAPSIALGFRDAADLLSPLALLRFSAVARGQDPPSGDSKDSTNPSVEPALPWASPGGALPCCGRLLSTRHLSPGLSSLRRLGLVYFLYLFLFSGLEYTLSFLTHQRFQFSSLQQGKMFFLIGLTMATIQGAYARRIHPGGEVAAVKRALLLLVPAFLLIGWGRSLPVLGLGLLLYSFAAAVVVPCLSSVVAGYGSPGQKGTVMGTLRSLGALARAAGPLVAASVYWLAGAQACFTTWSGLFLLPFFLLQKLSYPAQTLKAE

SEQ ID NO: 44 - DNA, 1476 bp, Homo sapiens
atgggctggggtggaggtggtggctgtactcctagacccccaattcatcaacaaccaccagaaagaagagtcgttaccgttgtttttctaggtttattattagacttgttggcttttaccttattgttacctttgttgcctggcttgttggaatctcatggtagagcccatgatccattgtatggttcttggcaaggtggcgttgattggtttgctacggctattggaatgccagttgaaaagcgttacaatagcgttttgtttggtggtttgattggtagcgcctttagtgttttacaatttttgtgtgctccattaacaggtgctacttccgattgtttgggtcgtagaccagtcatgttattgtgtttgatgggtgttgcaacttcttatgccgtctgggctacatcaagaagttttgctgcattcctcgcctctagattaattggtggtattagcaaaggtaatgtttctttgtctactgctattgtggctgatttgggttcacctttagctagatctcaaggtatggccgttattggtgttgctttttctttaggtttcacattgggtccaatgttgggtgcttctttaccattggaaatggctccttggtttgctttgttgtttgcagcttcagatttgttgtttatattttgtttcttaccagagacattgccattagaaaaacgcgctccatccattgctttgggttttagagatgctgctgatttattgtccccattagctttattgagattttctgctgttgctagaggtcaagatccaccatcaggtgactcaaaggattctactaatccatctgttgaaccagctttgccttgggcttctccaggaggtgctttaccatgttgtggtagattgttaagcacccgccatttatctccaggtttatcttcattgagaagattaggtttggtttattttctatatttgtttttattttctggtttggagtatactctatcctttttgactcaccaaagattccaattctctagcttacaacaaggtaaaatgttctttttaataggtttgacaatggccaccattcaaggtgcttacgctagaagaattcaccctggtggtgaagtcgctgcagttaaaagagctttgctgttgttggtcccagctttcttgttaattggttggggtcggtccttgccagttttgggtttgggtttgttattgtattcttttgctgccgcagttgtagttccatgtttgtcttcagtcgttgccggttacggtagtccaggtcaaaaaggtactgtcatgggtaccttgagatctttgggtgctctggctagggctgctggtcctttggttgctgcttctgtttattggttagccggcgcgcaagcttgttttactacttggtctggtctgttcttattaccattctttttgttgcaaaaattatcttaccctgctcagacattgaaagctgaataa

SEQ ID NO: 45 - protein, 519 aa, Saccharomyces cerevisiae
MPSTTLLFPQKHIRAIPGKIYAFFRELVSGVIISKPDLSHHYSCENATKEEGKDAADEEKTTTSLFPESNNIDRSLNGGCSVIPCSMDVSDLNTPISITLSPENRIKSEVNAKSLLGSRPEQDTGAPIKMSTGVTSSPLSPSGSTPEHSTKVLNNGEEEFICHYCDATFRIRGYLTRHIKKHAIEKAYHCPFFNSATPPDLRCHNSGGFSRRDTYKTHLKARHVLYPKGVKPQDRNKSSGHCAQCGEYFSTIENFVENHIESGDCKALPQGYTKKNEKRSGKLRKIKTSNGHSRFISTSQSVVEPKVLFNKDAVEAMTIVANNSSGNDIISKYGNNKLMLNSENFKVDIPKRKRKYIKKKQQQVSGSTVTTPEVATQNNQEVAPDEISSATIFSPFDTHLLEPVPSSSSESSAEVMFHGKQMKNFLIDINSFTNQQQQAQDNPSFLPLDIEQSSYDLSEDAMSYPIISTQSNRDCTQYDNTKISQILQSQLNPEYLSENHMRETQQYLNFYNDNFGSQF

SEQ ID NO: 46 - DNA, 1560 bp, Saccharomyces cerevisiae
atgccctctaccacgctactgtttccgcagaaacatattagggccattccaggcaagatatacgcgttcttcagagagctcgtcagcggagttattatatccaagccagatctaagtcatcattattcttgtgaaaatgcgacaaaggaggaaggcaaagatgcagcagatgaagaaaagactaccacaagtttgtttcccgaatcaaataatatagaccgttctttaaatggtggctgctctgtgatcccttgctccatggatgtcagcgatttgaacacgccaatatcgatcacactatctcctgagaatcgtatcaaatcagaagtaaatgccaagtcactgctcggatcaaggccagaacaagatacaggcgcccctatcaaaatgtctactggtgtcacaagctctccattaagtccatcaggctccaccccagaacattccaccaaggtcttgaacaacggcgaagaggagttcatttgtcactactgtgacgctactttcaggattagaggatatctaacgagacatattaagaagcacgccatcgaaaaggcgtatcactgtccattttttaacagtgccactcctcctgatcttagatgccacaattcgggtggttttagcagacgcgatacttataaaactcatttgaaggcaaggcacgtgctgtaccccaaaggtgttaaaccacaagaccgtaacaagtcgtccggccattgcgctcaatgtggtgaatacttttccaccattgaaaatttcgttgagaatcacattgagtctggggactgtaaagctttaccgcaggggtataccaagaaaaatgaaaaaagatctggaaaattaaggaagatcaagacatctaatggtcattctagattcatatccacttcgcaaagtgttgtagaacctaaagtacttttcaacaaggatgccgtagaggctatgaccatagtggctaataacagttcgggcaatgatattatatccaagtacggaaacaacaaattaatgttaaactcggaaaactttaaagtcgacatacccaagagaaagagaaaatatatcaagaaaaagcagcaacaggtatctggatcgacggtaaccacaccagaggtagctacacaaaacaatcaagaagtggcacctgatgaaatctcatccgccacaattttttcaccttttgatactcatctactcgagcccgtcccttctagttcatcggaatcttccgctgaagttatgttccatggcaagcaaatgaagaattttttgattgacataaacagcttcacaaatcaacagcagcaggcacaagataatccttcgttcctgccactcgatattgaacaatcttcatatgatttgagcgaagacgcaatgtcatatcccatcatatccacacaaagcaaccgtgattgcacgcagtatgataacacaaaaatctcacaaatcttacaatcacaactaaatccagaatatctcagcgaaaatcacatgagagagacacaacaatatttgaatttttacaatgacaactttgggtcacaattttga

SEQ ID NO: 47 - protein, 202 aa, Saccharomyces cerevisiae
MATNITWHPNLTYDERKALRKQDGCTIWLTGLSASGKSTIACALEQLLLQKNLSAYRLDGDNIRFGLNKDLGFSEKDRNENIRRISEVSKLFADSCAISITSFISPYRVDRDRARELHKEAGLKFIEIFVDVPLEVAEQRDPKGLYKKAREGVIKEFTGISAPYEAPKAPELHLRTDQKTVEECATIIYEYLISEKIIRKHL

SEQ ID NO: 48 - DNA, 609 bp, Saccharomyces cerevisiae
atggctactaatattacttggcatccaaatcttacttacgacgaacgcaaggcattgagaaaacaggacggttgtactatttggttaacaggtctaagtgcgtcaggtaaaagtacaatcgcctgtgcgctagaacagttactgctccaaaaaaacttgtctgcatatagattggatggtgacaacattcgttttggattgaacaaggatttgggtttctcagaaaaggacagaaatgaaaacattcgtagaattagcgaagtttctaagctatttgctgattcatgtgctatttcaatcacctcatttatctctccatacagagttgacagagatagagctcgtgaactacataaggaggctggtttgaagttcattgaaatatttgttgatgttccattagaagtcgctgagcaaagggaccctaagggtttatacaagaaagctagggagggtgtaatcaaggagtttacaggtatttctgccccatatgaagcgccaaaagctccagagctacatttgagaaccgaccagaagacggttgaagaatgtgctaccattatttatgagtacttaatcagtgaaaaaatcatccgtaagcatttgtaa

SEQ ID NO: 49 - protein, 261 aa, Saccharomyces cerevisiae
MKTYHLNNDIIVTQEQLDHWNEQLIKLETPQEIIAWSIVTFPHLFQTTAFGLTGLVTIDMLSKLSEKYYMPELLFIDTLHHFPQTLTLKNEIEKKYYQPKNQTIHVYKPDGCESEADFASKYGDFLWEKDDDKYDYLAKVEPAHRAYKELHISAVFTGRRKSQGSARSQLSIIEIDELNGILKINPLINWTFEQVKQYIDANNVPYNELLDLGYRSIGDYHSTQPVKEGEDERAGRWKGKAKTECGIHEASRFAQFLKQDA

SEQ ID NO: 50 - DNA, 786 bp, Saccharomyces cerevisiae
atgaagacctatcatttgaataatgatataattgtcacacaagaacagttggatcattggaatgaacaactaatcaagctggaaacgccacaggagattattgcatggtctatcgtaacgtttcctcaccttttccaaaccactgcatttggtttgactggcttggttactatcgatatgttgtcaaagctatctgaaaaatactacatgccagaactattatttatagacactttgcaccatttcccacaaactttaacactaaaaaacgagattgagaaaaaatactaccagcctaaaaatcaaaccattcacgtatataagccggatggatgtgaatcggaggcagattttgcctcgaaatacggggatttcttatgggagaaagatgatgacaagtacgattatctggccaaagtggaacctgcacatcgtgcctacaaagagctacatataagtgctgtgtttactggtagaagaaaatcacaaggttctgcccgctcccaactgtcgattattgaaatagacgaacttaatggaatcttaaaaataaatccattgatcaattggacgttcgagcaggttaaacagtatatagatgcaaacaatgtaccatacaacgaacttttggaccttggatatagatccattggtgattaccattccacacaacccgtcaaggaaggtgaagatgagagagcaggaagatggaagggcaaggccaagaccgagtgtggaattcatgaagccagccgattcgcgcaatttttaaagcaagatgcctag

SEQ ID NO: 51 - DNA, 3801 bp, Saccharomyces cerevisiae
atgtcgaatataagtaccaaagatatacgaaaaagtaagccaaaaagaggatccggcttcgatttacttgaagtgactgaatcacttggctatcagacacacaggaaaaatggaagaaactcatggtcaaaggacgacgataatatgctacgatcgctagtaaacgaatccgcaaaggagttaggctatgagaatggacttgaagacgtaaaaacaattcaacaatccaaccatctttctaaatgtattgcttgggatgttttagctacacgattcaaacacaccgtaagaacttcaaaggatgtaagaaagcgttggacaggatctctggacccaaacttgaagaaaggtaaatggacacaagaagaggatgagcagctcttgaaagcttatgaagaacatgggcctcactggctgagtatctccatggatatccctggaagaacagaggatcaatgtgcgaaaaggtacattgaagtgttgggacctggaagtaaaggtagattaagagaatggacgctcgaggaagatttaaatctgataagtaaagtgaaggcatacggcacgaaatggagaaagatttcgtcggaaatggaattcagaccaagtttaacgtgcaggaacagatggcggaaaattattactatggttgtgcgaggacaagcatcagaagtaataacaaaggctataaaagaaaacaagaacatagacatgacagacggaaaactccgacaacatccaatcgccgattctgatatacgctcagattctacaccgaataaagaagaacagttgcagctatctcaacagaacaacccttcattgataaagcaggatatattgaatgttaaagagaacgaatcgagcaaactaccaagattaaaggataatgatggacccattttaaatgacagcaagccacaagcattacctcccttaaaagagatatctgcacctccaccaatcaggatgacgcaagtcggtcagactcacaccagtggtagcattaggagcaaagtatctttaccaattgaaggtctttctcaaatgaacaagcaaagtcctggaggaatctcagatagcccacagacaagccttcccccagcattcaatccagcatctctcgatgagcatatgatgaatagtaatagtatttcagattctccgaagcacgcctattctactgtgaaaactagagaaccaaattcctctagcacacaatggaaatttacattaaaagatggtcaaggtttgtcaatttctaacggaactattgacagcacaaaattagtaaaagaattggttgaccaagctaagaagtactctttaaaaatttcgatacatcaacatattcacaatcattatgttacatccacagaccatcctgtcagcagcaatactggattgtcaaatataggaaatataaatggaaaccctctattaatggatagtttcccacatatgggtagacaactgggaaatggtcttccagggttaaattcaaatagtgacacttttaacccagaatataggacttctctggacaacatggatagcgattttttatcaagaacacccaactataacgcattcagcttggaagcaacttcacacaatcccgctgacaatgcaaatgaacttggttcacaaagcaatagagaaacaaacagcccgtctgtattttatcctcaagcaaatacattgatcccaactaattctaccgctacaaataatgaaatcattcaaggaaacgttagtgctaatagtatgtcaccgaatttcaatggaacaaatggcaaggcaccaagttcgacggcatcatatacaacgagcggttcagaaatgccacctgatgtaggtcctaatagaatagcgcatttcaactatttgccaccaactatacggcctcatttaggctcatcagatgcgacaagaggtgctgacttgaataagttactcaatccatctccaaattcagtaagaagtaacggcagtaaaactaagaaaaaagaaaaaagaaaaagtgaatcatctcagcatcattctacatcatctgtgacaatcaataaattcaatcacattgatcagtcggagatatcaaggactacatccagatcccccgggatgatggaagaattctcgtacgatcacgattttaacacacattttgctacagatttggattatttgcaacatgaccaacaacaacaacagcaacaacatgatcaacaacataatcaacagcaacaaccacaaccacaaccaattcaaactcaaaacctggagcacgaccacgaccaacatactaatgatatgagtgcttcatcgaatgcatcagatagtggacctcagaggcccaagaggactcgcgcaaagggtgaagcactagatgtcctaaagcgtaaatttgaaataaatccaacaccctctttggtagaaagaaagaaaatatcagatctgataggaatgcctgaaaaaaacgtcagaatttggtttcagaacagaagagctaaattgcggaaaaagcagcatggaagtaataaggacacaatcccctcgtcacaatcccgtgatattgccaacgattacgaacgtgggagtacagacaacaatttggtcactacaacaagtacttcatccatatttcacgatgaagacctgacttttttcgaccgtattccgttgaacagcaacaacaactattatttttttgacatttgctcaattactgtgggaagttggaatagaatgaaaagcggcgcactgcaaagaaggaactttcagtctataaaggagttgagaaacctatcgccaataaagattaataacataatgtcgaatgccacagatttaatggttttgatatccaagaaaaactcagaaataaactatttttttagtgctatggcaaataatactaaaattctcttcaggatctttttcccattaagttcagtcacgaattgctctctaactttagaaactgacgacgatataataaatagtaacaacacaagcgataaaaacaatagtaatactaataatgatgatgataacgacgataacagtaacgaagacaatgataatagtagtgaggataagaggaatgctaaggataactttggagaattgaagctaacagtcaccagatcacccacttttgctgtttactttttaaataatgctcctgatgaagatccaaatttgaacaatcagtggtccatatgtgatgatttctcagaaggtagacaggtaaatgacgcatttgttggtggttcgaatattcctcacactttgaaaggtttacagaaatcattaagattcatgaattctctaattctagactataaatcatcgaatgaaatattacctacgatcaatacagcgatccccactgctgcagttccacaacagaatattgcccctccctttctgaatacaaattcaagtgcaacagactcaaatccaaatacaaatttagaagattctctcttcttcgatcatgatctgttatcgagttcgataaccaacaccaacaacggacaaggctctaataatggacgtcaagctagcaaggatgatacgctcaatttactggatactaccgtcaacagcaataacaatcataatgctaataatgaggagaatcatctagcgcaagaacatttatccagcgatgctgatattgttgcaaatccaaatgatcatttgttgtctttaccgactgatagtgaacttccaaatactccagattttttgaagaacactaacgaactaactgacgagcatagatggatatga